(19) United States
(12) Patent Application Publication
(10) Pub. No.: US 2020/0078406 A1
(43) Pub. Date: Mar. 12, 2020

(54) SYSTEMS AND METHODS FOR PREPARATION OF PLATELETS

(71) Applicant: Cerus Corporation, Concord, CA (US)

(72) Inventors: Elan WEINER, Concord, CA (US); William GREENMAN, Concord, CA (US)

(73) Assignee: Cerus Corporation, Concord, CA (US)

(21) Appl. No.: 16/068,650

(22) PCT Filed: Jan. 6, 2017
(86) PCT No.: PCT/US2017/012633

§ 371 Date: Jul. 6, 2018

Related U.S. Application Data
(60) Provisional application No. 62/276,223, filed on Jan. 7, 2016.
(60) Provisional application No. 62/311,373, filed on Mar. 21, 2016.

(51) Int. Cl. A61K 35/19; A61M 1/02; A61M 1/38

(52) U.S. Cl. CPC A61K 35/19; A61M 1/0281; A61M 1/3693; A61M 1/387; A61M 1/385

(57) ABSTRACT

Provided are methods, systems and compositions for the preparation of platelets, including improved methods and systems for preparing apheresis derived platelet units suitable for infusion and compositions comprising such platelet units.

### CROSS REFERENCE TO RELATED APPLICATIONS

This application claims the priority benefit of U.S. Provisional Application Ser. No. 62/276,223, filed Jan. 7, 2016, and 62/311,373, filed Mar. 21, 2016, each of which is incorporated herein by reference in its entirety.

### TECHNICAL FIELD

The methods, systems and compositions described herein generally relate to the preparation of platelets. More particularly the present disclosure relates to improved methods and systems for preparing apheresis derived platelet units suitable for infusion and compositions comprising such platelet units.

### BACKGROUND

Blood component collection and processing serves a critical role in healthcare worldwide, and millions of units of donated blood components are collected by blood banks each year. While some units of whole blood are collected from donors and used directly for transfusion, most donations are generally separated into the blood components red blood cells, platelets and plasma for more specific therapeutic use. Separation may be either following collection of whole blood donations or at the point of collection if using a suitable separation device system, such as an apheresis collection device. Individual blood components are then used in treating different medical needs and conditions based on therapeutic need.

Platelets play a key role in hemostasis, clot stability and retraction, as well as in vascular repair and anti-microbial host defense. A variety of methods are used to collect and store platelet blood products for clinical use. Collection of platelets from donated whole blood donation is generally in the form of platelet concentrates (PC), obtained using processing methods such as a buffy coat or platelet rich plasma method, and such PC may be pooled to generate a platelet unit of sufficient therapeutic dosage for transfusion. In general, PC from four to six individual donors of compatible blood types must be combined to produce a single platelet unit of sufficient therapeutic dosage for transfusion.

Apheresis collection provides a method to obtain platelet units of sufficient therapeutic dosage from a single donor, without the need for pooling and any related risk from the infusion recipient's exposure to platelets from multiple donors. Apheresis collection of platelets utilizes an automated device that separates platelets from the donor blood and returns remaining blood components (e.g., red blood cells, plasma) to the donor during the donation process. Apheresis platelet donations are generally based on certain donor parameters, such as gender, physical size (e.g., weight), hemoglobin level, platelet count on the day of donation and donation frequency, in part to ensure only a safe amount is collected. From these parameters, apheresis platelet donations generally are collected from an individual donor as a volume to yield one, two or three platelet units each containing a specified minimum number (e.g., at least a minimum number) of platelets per unit to meet the therapeutic dose requirement, with such per unit or therapeutic dose criteria generally determined by governmental, regulatory, institution or accrediting organization (e.g., industry) standards. Blood centers may further determine their own apheresis platelet collection specifications for singles, doubles and triples collections (e.g., split points), including for example, the amount (e.g., number) of excess platelets over that required to meet governmental, regulatory, institution or accrediting organization standards. Generally platelet collections to achieve the specified threshold for one, two or three platelet units typically factor in a number of variables that result in a distribution of platelet yields (e.g., bell curve distribution), but do not need to be collected in excess from a donor in apheresis procedures, other than to account for anticipated donor and/or process variability, collection time limitations (e.g., based on donor, based on blood center operations) splitting and testing (e.g., Bac-T testing), as excess platelets would be retained in the one, two or three units, but without providing additional value from a platelet unit production standpoint. Thus, even if a donor could safely donate an amount of platelets by apheresis corresponding to, for example, 1.6 platelet units or 2.8 platelet units, there is generally no benefit or incentive for a blood center to collect such an amount, as the platelet units ultimately produced and provided to a doctor or hospital would be simply 1 platelet unit or 2 platelet units, respectively.

There remains a need for improved systems and methods that provide greater efficiency for collection and preparation of apheresis platelet units, including pathogen-inactivated platelet units, in order to more fully capture the overall potential for production of platelet units, such as in blood centers using apheresis collection systems for platelets.

### SUMMARY

The methods, systems and compositions described herein are useful for improved collection and preparation of platelet units (e.g., apheresis-derived platelet units) suitable for infusion (e.g., into a subject), and compositions comprising such platelet units, such as therapeutic dosage units of platelets.

In one aspect, the present disclosure provides a method of preparing a plurality of platelet units suitable for infusion (e.g., suitable for infusion into a subject), comprising: a) collecting a first platelet donation by apheresis from a first donor, b) collecting a second platelet donation by apheresis from a second donor, c) combining the first platelet donation and the second platelet donation to yield a pooled platelet preparation, and d) separating the pooled platelet preparation into a plurality of platelet units (e.g., therapeutic doses, therapeutic dosage units) each in an individual container, wherein the plurality of platelet units comprises a greater number of platelet units than the total number of platelet units that can be prepared from the first platelet donation and second platelet donation without combining the first and second platelet donations.

In some embodiments, the first platelet donation comprises less than the number (e.g., less than the minimum number, less than the minimum quantity) of platelets required to prepare one platelet unit. In some embodiments, the platelet donation comprises an amount (e.g., number) of platelets that is less than about 99%, 98%, 95%, 90%, 85%, 80%, 75%, 70%, 65%, 60%, 55%, 50%, 45%, 40%, 35%, 30% or about 25% of the number of platelets required to prepare one platelet unit. In some embodiments, the platelet donation comprises an amount of platelets that is at least 10% of the number of platelets required to prepare one platelet unit. In some embodiments, the platelet donation comprises an amount of platelets that is about 10% to about 98%, about 15% to about 98%, about 20% to about 98%, about 25% to about 98%, about 30% to about 98%, about 35% to about 98%, about 40% to about 98% or about 50% to about 98% of the number of platelets required to prepare one platelet unit. In some embodiments, the platelet donation comprises an amount of platelets that is about 98%, 95%, 90%, 85%, 80%, 75%, 70%, 65%, 60%, 55%, 50%, 45%, 40%, 35%, 30% or about 25% of the number of platelets required to prepare one platelet unit. In some embodiments, collecting the first platelet donation from the first donor comprises targeting the platelet collection to yield a first platelet donation comprising less than about 99%, 98%, 95%, 90%, 85%, 80%, 75%, 70%, 65%, 60%, 55%, 50%, 45%, 40%, 35%, 30% or less than about 25% of the number of platelets required to prepare one platelet unit.

In some embodiments, the first platelet donation comprises greater than the number (e.g., greater than the minimum number, greater than the minimum quantity) of platelets required to prepare one platelet unit, but less than the number (e.g., less than the minimum number, less than the minimum quantity) of platelets required to prepare two platelet units. In some embodiments, greater than the number of platelets required to prepare one platelet unit, but less than the number of platelets required to prepare two platelet units is about 1.1-fold, 1.2-fold, 1.3-fold, 1.4-fold, 1.5-fold, 1.6-fold, 1.7-fold, 1.8-fold, 1.9-fold, 1.95-fold or about 1.98-fold the number of platelets required to prepare one platelet unit. In some embodiments, greater than the number of platelets required to prepare one platelet unit, but less than the number of platelets required to prepare two platelet units is about 1.1-fold to about 1.98-fold, about 1.15-fold to about 1.98-fold, about 1.2-fold to about 1.98-fold, about 1.25-fold to about 1.98-fold, 1.3-fold to about 1.98-fold, about 1.35-fold to about 1.98-fold, about 1.4-fold to about 1.98-fold or about 1.5-fold to about 1.98-fold the number of platelets required to prepare one platelet unit. In some embodiments, greater than the number of platelets required to prepare one platelet unit, but less than the number of platelets required to prepare two platelet units is about 105%, 110%, 115%, 120%, 125%, 130%, 135%, 140%, 145%, 150%, 155%, 160%, 165%, 170%, 175%, 180%, 185%, 190% or about 195% the number of platelets required to prepare one platelet unit. In some embodiments, greater than the number of platelets required to prepare one platelet unit, but less than the number of platelets required to prepare two platelet units is about 110% to about 198%, about 115% to about 198%, about 120% to about 198%, about 125% to about 198%, about 130% to about 198%, about 135% to about 198%, about 140% to about 198% or about 150% to about 198% the number of platelets required to prepare one platelet unit. In some embodiments, collecting the first platelet donation from the first donor comprises targeting the platelet collection to yield a first platelet donation comprising at least about 125%, 130%, 135%, 140%, 145%, 150%, 155%, 160%, 165%, 170%, 175%, 180%, 185%, 190% or about 195% of the number of platelets required to prepare one platelet unit, but less than the number of platelets required to prepare two platelet units.

In some embodiments, the first platelet donation comprises greater than the number of platelets required to prepare two platelet units, but less than the number of platelets required to prepare three platelet units. In some embodiments, greater than the number of platelets required to prepare two platelet units, but less than the number of platelets required to prepare three platelet units is about 1.1-fold, 1.2-fold, 1.3-fold, 1.4-fold, 1.5-fold, 1.6-fold, 1.7-fold, 1.8-fold, 1.9-fold, 1.95-fold or about 1.98-fold the number of platelets required to prepare two platelet units. In some embodiments, greater than the number of platelets required to prepare two platelet units, but less than the number of platelets required to prepare three platelet units is about 1.1-fold to about 1.98-fold, about 1.15-fold to about 1.98-fold, about 1.2-fold to about 1.98-fold, about 1.25-fold to about 1.98-fold, about 1.3-fold to about 1.98-fold, about 1.35-fold to about 1.98-fold, about 1.4-fold to about 1.98-fold or about 1.5-fold to about 1.98-fold the number of platelets required to prepare two platelet units. In some embodiments, greater than the number of platelets required to prepare two platelet units, but less than the number of platelets required to prepare three platelet units is about 105%, 110%, 115%, 120%, 125%, 130%, 135%, 140%, 145%, 150%, 155%, 160%, 165%, 170%, 175%, 180%, 185%, 190% or about 195% the number of platelets required to prepare two platelet units. In some embodiments, greater than the number of platelets required to prepare two platelet units, but less than the number of platelets required to prepare three platelet units is about 110% to about 198%, about 115% to about 198%, about 120% to about 198%, about 125% to about 198%, about 130% to about 198%, about 135% to about 198%, about 140% to about 198% or about 150% to about 198% the number of platelets required to prepare two platelet units. In some embodiments, collecting the first platelet donation from the first donor comprises targeting the platelet collection to yield a first platelet donation comprising at least about 225%, 230%, 235%, 240%, 245%, 250%, 255%, 260%, 265%, 270%, 275%, 280%, 285%, 290% or at least about 295% of the number of platelets required to prepare one platelet unit, but less than the number of platelets required to prepare three platelet units. In some embodiments, the first platelet donation comprises about 210% to about 298%, about 215% to about 298%, about 220% to about 298%, about 225% to about 298%, about 230% to about 298%, about 235% to about 298%, about 240% to about 298%, about 245% to about 298%, or about 250% to about 298% of the number of platelets required to prepare one platelet unit.

In some embodiments, the first platelet donation comprises greater than the number of platelets required to prepare three platelet units, but less than the number of platelets required to prepare four platelet units. In some embodiments, greater than the number of platelets required to prepare three platelet units, but less than the number of platelets required to prepare four platelet units is about 1.1-fold, 1.2-fold, 1.3-fold, 1.4-fold, 1.5-fold, 1.6-fold, 1.7-fold, 1.8-fold, 1.9-fold, 1.95-fold or about 1.98-fold the number of platelets required to prepare three platelet units. In some embodiments, greater than the number of platelets required to prepare three platelet units, but less than the number of platelets required to prepare four platelet units is about 1.1-fold to about 1.98-fold, about 1.15-fold to about 1.98-fold, about 1.2-fold to about 1.98-fold, about 1.25-fold to about 1.98-fold, about 1.3-fold to about 1.98-fold, about 1.35-fold to about 1.98-fold, about 1.4-fold to about 1.98-fold or about 1.5-fold to about 1.98-fold the number of platelets required to prepare three platelet units. In some embodiments, greater than the number of platelets required to prepare three platelet units, but less than the number of platelets required to prepare four platelet units is about 105%, 110%, 115%, 120%, 125%, 130%, 135%, 140%, 145%, 150%, 155%, 160%, 165%, 170%, 175%, 180%, 185%, 190% or about 195% the number of platelets required to prepare three platelet units. In some embodiments, greater than the number of platelets required to prepare three platelet units, but less than the number of platelets required to prepare four platelet units is about 110% to about 198%, about 115% to about 198%, about 120% to about 198%, about 125% to about 198%, about 130% to about 198%, about 135% to about 198%, about 140% to about 198% or about 150% to about 198% the number of platelets required to prepare three platelet units. In some embodiments, collecting the first platelet donation from the first donor comprises targeting the platelet collection to yield a first platelet donation comprising at least about 325%, 330%, 335%, 340%, 345%, 350%, 355%, 360%, 365%, 370%, 375%, 380%, 385%, 390% or at least about 395% of the number of platelets required to prepare one platelet unit, but less than the number of platelets required to prepare three platelet units. In some embodiments, the first platelet donation comprises about 310% to about 398%, about 315% to about 398%, about 320% to about 398%, about 325% to about 398%, about 330% to about 398%, about 335% to about 398%, about 340% to about 398%, about 345% to about 398%, or about 350% to about 398% of the number of platelets required to prepare one platelet unit.

In some embodiments, the second platelet donation comprises greater than the number of platelets required to prepare one platelet unit, but less than the number of platelets required to prepare two platelet units. In some embodiments, greater than the number of platelets required to prepare one platelet unit, but less than the number of platelets required to prepare two platelet units is about 1.1-fold, 1.2-fold, 1.3-fold, 1.4-fold, 1.5-fold, 1.6-fold, 1.7-fold, 1.8-fold, 1.9-fold, 1.95-fold or about 1.98-fold the number of platelets required to prepare one platelet unit. In some embodiments, greater than the number of platelets required to prepare one platelet unit, but less than the number of platelets required to prepare two platelet units is about 1.1-fold to about 1.98-fold, about 1.15-fold to about 1.98-fold, about 1.2-fold to about 1.98-fold, about 1.25-fold to about 1.98-fold, about 1.3-fold to about 1.98-fold, about 1.35-fold to about 1.98-fold, about 1.4-fold to about 1.98-fold or about 1.5-fold to about 1.98-fold the number of platelets required to prepare one platelet unit. In some embodiments, greater than the number of platelets required to prepare one platelet unit, but less than the number of platelets required to prepare two platelet units is about 105%, 110%, 115%, 120%, 125%, 130%, 135%, 140%, 145%, 150%, 155%, 160%, 165%, 170%, 175%, 180%, 185%, 190% or about 195% the number of platelets required to prepare one platelet unit. In some embodiments, greater than the number of platelets required to prepare one platelet unit, but less than the number of platelets required to prepare two platelet units is about 110% to about 198%, about 115% to about 198%, about 120% to about 198%, about 125% to about 198%, about 130% to about 198%, about 135% to about 198%, about 140% to about 198% or about 150% to about 198% the number of platelets required to prepare one platelet unit. In some embodiments, collecting the second platelet donation from the second donor comprises targeting the platelet collection to yield a second platelet donation comprising at least about 125%, 130%, 135%, 140%, 145%, 150%, 155%, 160%, 165%, 170%, 175%, 180%, 185%, 190% or about 195% of the number of platelets required to prepare one platelet unit, but less than the number of platelets required to prepare two platelet units. In some embodiments, collecting the second platelet donation from the second donor comprises targeting the platelet collection to yield a second platelet donation comprising a sufficient number of platelets to prepare two platelet units, to prepare three platelet units, to prepare four platelet units, or to prepare five platelet units after combining the first and second platelet donations and separating the resulting pooled platelet preparation into a plurality of platelet units. In some embodiments, the second platelet donation comprises a sufficient number of platelets that, when combined with the first platelet donation, yields a pooled platelet preparation comprising a sufficient number of platelets to separate into two, into three, into four, or into five platelet units. In some embodiments, collecting the second platelet donation from the second donor comprises collecting a sufficient number of platelets to prepare two platelet units, to prepare three platelet units, to prepare four platelet units, or to prepare five platelet units from the pooled preparation.

In some embodiments, the second platelet donation comprises greater than the number of platelets required to prepare two platelet units, but less than the number of platelets required to prepare three platelet units. In some embodiments, greater than the number of platelets required to prepare two platelet units, but less than the number of platelets required to prepare three platelet units is about 1.1-fold, 1.2-fold, 1.3-fold, 1.4-fold, 1.5-fold, 1.6-fold, 1.7-fold, 1.8-fold, 1.9-fold, 1.95-fold or about 1.98-fold the number of platelets required to prepare two platelet units. In some embodiments, greater than the number of platelets required to prepare two platelet units, but less than the number of platelets required to prepare three platelet units is about 1.1-fold to about 1.98-fold, about 1.15-fold to about 1.98-fold, about 1.2-fold to about 1.98-fold, about 1.25-fold to about 1.98-fold, about 1.3-fold to about 1.98-fold, about 1.35-fold to about 1.98-fold, about 1.4-fold to about 1.98-fold or about 1.5-fold to about 1.98-fold the number of platelets required to prepare two platelet units. In some embodiments, greater than the number of platelets required to prepare two platelet units, but less than the number of platelets required to prepare three platelet units is about 105%, 110%, 115%, 120%, 125%, 130%, 135%, 140%, 145%, 150%, 155%, 160%, 165%, 170%, 175%, 180%, 185%, 190% or about 195% the number of platelets required to prepare two platelet units. In some embodiments, greater than the number of platelets required to prepare two platelet units, but less than the number of platelets required to prepare three platelet units is about 110% to about 198%, about 115% to about 198%, about 120% to about 198%, about 125% to about 198%, about 130% to about 198%, about 135% to about 198%, about 140% to about 198% or about 150% to about 198% the number of platelets required to prepare two platelet units. In some embodiments, greater than the number of platelets required to prepare two platelet units, but less than the number of platelets required to prepare three platelet units is about 210% to about 298%, about 215% to about 298%, about 220% to about 298%, about 225% to about 298%, about 230% to about 298%, about 235% to about 298%, about 240% to about 298% or about 250% to about 298% the number of platelets required to prepare one platelet unit. In some embodiments, collecting the second platelet donation from the second donor comprises targeting the platelet collection to yield a second platelet donation comprising at least about 225%, 230%, 235%, 240%, 245%, 250%, 255%, 260%, 265%, 270%, 275%, 280%, 285%, 290% or about 295% of the number of platelets required to prepare one platelet unit, but less than the number of platelets required to prepare three platelet units. In some embodiments, collecting the second platelet donation from the second donor comprises targeting the platelet collection to yield a second platelet donation comprising a sufficient number of platelets to prepare three platelet units, to prepare four platelet units, to prepare five platelet units, or to prepare six platelet units after combining the first and second platelet donations and separating the resulting pooled platelet preparation into a plurality of platelet units. In some embodiments, the second platelet donation comprises a sufficient number of platelets that, when combined with the first platelet donation, yields a pooled platelet preparation comprising a sufficient number of platelets to separate into three, into four, into five, or into six platelet units. In some embodiments, collecting the second platelet donation from the second donor comprises collecting a sufficient number of platelets to prepare three platelet units, to prepare four platelet units, to prepare five platelet units, or to prepare six platelet units from the pooled preparation.

In some embodiments, the second platelet donation comprises greater than the number of platelets required to prepare three platelet units, but less than the number of platelets required to prepare four platelet units. In some embodiments, greater than the number of platelets required to prepare three platelet units, but less than the number of platelets required to prepare four platelet units is about 1.1-fold, 1.2-fold, 1.3-fold, 1.4-fold, 1.5-fold, 1.6-fold, 1.7-fold, 1.8-fold, 1.9-fold, 1.95-fold or about 1.98-fold the number of platelets required to prepare three platelet units. In some embodiments, greater than the number of platelets required to prepare three platelet units, but less than the number of platelets required to prepare four platelet units is about 1.1-fold to about 1.98-fold, about 1.15-fold to about 1.98-fold, about 1.2-fold to about 1.98-fold, about 1.25-fold to about 1.98-fold, about 1.3-fold to about 1.98-fold, about 1.35-fold to about 1.98-fold, about 1.4-fold to about 1.98-fold or about 1.5-fold to about 1.98-fold the number of platelets required to prepare three platelet units. In some embodiments, greater than the number of platelets required to prepare three platelet units, but less than the number of platelets required to prepare four platelet units is about 105%, 110%, 115%, 120%, 125%, 130%, 135%, 140%, 145%, 150%, 155%, 160%, 165%, 170%, 175%, 180%, 185%, 190% or about 195% the number of platelets required to prepare three platelet units. In some embodiments, greater than the number of platelets required to prepare three platelet units, but less than the number of platelets required to prepare four platelet units is about 110% to about 198%, about 115% to about 198%, about 120% to about 198%, about 125% to about 198%, about 130% to about 198%, about 135% to about 198%, about 140% to about 198% or about 150% to about 198% the number of platelets required to prepare three platelet units. In some embodiments, greater than the number of platelets required to prepare two platelet units, but less than the number of platelets required to prepare three platelet units is about 310% to about 398%, about 315% to about 398%, about 320% to about 398%, about 325% to about 398%, about 330% to about 398%, about 335% to about 398%, about 340% to about 398% or about 350% to about 398% the number of platelets required to prepare one platelet unit. In some embodiments, collecting the second platelet donation from the second donor comprises targeting the platelet collection to yield a second platelet donation comprising at least about 325%, 330%, 335%, 340%, 345%, 350%, 355%, 360%, 365%, 370%, 375%, 380%, 385%, 390% or about 395% of the number of platelets required to prepare one platelet unit, but less than the number of platelets required to prepare four platelet units. In some embodiments, collecting the second platelet donation from the second donor comprises targeting the platelet collection to yield a second platelet donation comprising a sufficient number of platelets to prepare four platelet units, to prepare five platelet units, to prepare six platelet units, or to prepare seven platelet units after combining the first and second platelet donations and separating the resulting pooled platelet preparation into a plurality of platelet units. In some embodiments, the second platelet donation comprises a sufficient number of platelets that, when combined with the first platelet donation, yields a pooled platelet preparation comprising a sufficient number of platelets to separate into four, into five, into six, or into seven platelet units. In some embodiments, collecting the second platelet donation from the second donor comprises collecting a sufficient number of platelets to prepare four platelet units, to prepare five platelet units, to prepare six platelet units, or to prepare seven platelet units from the pooled preparation.

In some embodiments, the first platelet donation comprises less than the number of platelets required to prepare one platelet unit and the second platelet donation comprises greater than the number of platelets required to prepare one platelet unit, but less than the number of platelets required to prepare two platelet units. In some embodiments, the first platelet donation comprises less than the number of platelets required to prepare one platelet unit and the second platelet donation comprises greater than the number of platelets required to prepare two platelet units, but less than the number of platelets required to prepare three platelet units. In some embodiments, the first platelet donation comprises less than the number of platelets required to prepare one platelet unit and the second platelet donation comprises greater than the number of platelets required to prepare three platelet units, but less than the number of platelets required to prepare four platelet units. In some embodiments, the pooled platelet preparation comprises a sufficient number of platelets to prepare two platelet units, to prepare three platelet units, or to prepare four platelet units. In some embodiments, the plurality of platelet units comprises 2 platelet units, 3 platelet units or 4 platelet units.

In some embodiments, the first platelet donation comprises greater than the number of platelets required to prepare one platelet unit, but less than the number of platelets required to prepare two platelet units, and the second platelet donation comprises greater than the number of platelets required to prepare one platelet unit, but less than the number of platelets required to prepare two platelet units. In some embodiments, the first platelet donation comprises greater than the number of platelets required to prepare one platelet unit, but less than the number of platelets required to prepare two platelet units, and the second platelet donation comprises greater than the number of platelets required to prepare two platelet units, but less than the number of platelets required to prepare three platelet units. In some embodiments, the first platelet donation comprises greater than the number of platelets required to prepare one platelet unit, but less than the number of platelets required to prepare two platelet units, and the second platelet donation comprises greater than the number of platelets required to prepare three platelet units, but less than the number of platelets required to prepare four platelet units. In some embodiments, the pooled platelet preparation comprises a sufficient number of platelets to prepare three platelet units, to prepare four platelet units, or to prepare five platelet units. In some embodiments, the plurality of platelet units comprises 3 platelet units, 4 platelet units or 5 platelet units.

In some embodiments, the first platelet donation comprises greater than the number of platelets required to prepare two platelet units, but less than the number of platelets required to prepare three platelet units, and the second platelet donation comprises greater than the number of platelets required to prepare two platelet units, but less than the number of platelets required to prepare three platelet units. In some embodiments, the first platelet donation comprises greater than the number of platelets required to prepare two platelet units, but less than the number of platelets required to prepare three platelet units, and the second platelet donation comprises greater than the number of platelets required to prepare three platelet units, but less than the number of platelets required to prepare four platelet units. In some embodiments, the first platelet donation comprises greater than the number of platelets required to prepare three platelet units, but less than the number of platelets required to prepare four platelet units, and the second platelet donation comprises greater than the number of platelets required to prepare three platelet units, but less than the number of platelets required to prepare four platelet units. In some embodiments, the pooled platelet preparation comprises a sufficient number of platelets to prepare five platelet units, to prepare six platelet units, or to prepare seven platelet units. In some embodiments, the plurality of platelet units comprises 5 platelet units, 6 platelet units or 7 platelet units.

In some of the aforementioned embodiments, the method further comprises collecting a third platelet donation by apheresis from a third donor, and combining the third platelet donation with the first and the second platelet donations to yield a pooled platelet preparation, and separating the pooled platelet preparation into a plurality of platelet units each in an individual container, wherein the plurality of platelet units comprises a greater number of platelet units than the total number of platelet units that can be prepared from the first platelet donation, the second platelet donation and the third platelet donation without combining the first, second and third platelet donations. In some embodiments, the third platelet donation comprises greater than the number of platelets required to prepare one platelet unit, but less than the number of platelets required to prepare two platelet units. In some embodiments, the third platelet donation comprises greater than the number of platelets required to prepare two platelet units, but less than the number of platelets required to prepare three platelet units. In some embodiments, the third platelet donation comprises greater than the number of platelets required to prepare three platelet units, but less than the number of platelets required to prepare four platelet units.

In some of the aforementioned embodiments, the second platelet donation is collected less than about (e.g., within) 24 hours, 22 hours, 20 hours, 16 hours, 12 hours, 10 hours, 8 hours, 6 hours, 5 hours, 4 hours, 3 hours, 2 hours or less than about 1 hour from the time the first platelet donation is collected. In some embodiments, the second platelet donation is collected about the same time (e.g., overlapping collection time) as the first platelet donation. In some embodiments, the second platelet donation is collected within about 7 days, within about 6 days, within about 5 days, within about 4 days, within about 3 days, within about 2 days, or within about 1 day from the time the first platelet donation is collected. In some embodiments, the second platelet donation is collected within 20 hours from the time the first platelet donation is collected. In some embodiments, the second platelet donation is collected within 12 hours from the time the first platelet donation is collected. In some embodiments, the second platelet donation is collected within 6 hours from the time the first platelet donation is collected. In some of the aforementioned embodiments, the third platelet donation is collected less than about (e.g., within) 24 hours, 22 hours, 20 hours, 16 hours, 12 hours, 10 hours, 8 hours, 6 hours, 5 hours, 4 hours, 3 hours, 2 hours or less than about 1 hour from the time the first platelet donation is collected. In some embodiments, the third platelet donation is collected about the same time (e.g., overlapping collection time) as the first platelet donation. In some embodiments, the third platelet donation is collected within about 7 days, within about 6 days, within about 5 days, within about 4 days, within about 3 days, within about 2 days, or within about 1 day from the time the first platelet donation is collected. In some embodiments, the third platelet donation is collected within 20 hours from the time the first platelet donation is collected. In some embodiments, the third platelet donation is collected within 12 hours from the time the first platelet donation is collected. In some embodiments, the third platelet donation is collected within 6 hours from the time the first platelet donation is collected. In some embodiments, the first platelet donation, the second platelet donation and the third platelet donation are collected within a period (e.g., the same period) of about 24 hours, 22 hours, 20 hours, 16 hours, 12 hours, 10 hours, 8 hours, 6 hours, 5 hours, 4 hours, 3 hours, 2 hours or about 1 hour. In some embodiments, the first platelet donation, the second platelet donation and the third platelet donation are collected at about the same time (e.g., overlapping collection times). In some embodiments, the first platelet donation, the second platelet donation and the third platelet donation are collected within about 7 days, within about 6 days, within about 5 days, within about 4 days, within about 3 days, within about 2 days, within about 1 day, within about 72 hours, within about 48 hours, within about 36 hours, within about 24 hours, within about 20 hours, within about 12 hours or within about 6 hours of each other. In some of the aforementioned embodiments, the pooled platelet preparation comprises platelets only from donors (e.g., first donor, second donor, third donor) of the same ABO blood type.

In some of the aforementioned embodiments, the method further comprises subjecting the first platelet donation, the second platelet donation, and/or the third platelet donation to a pathogen inactivation process. In some embodiments, the method further comprises subjecting each of the first platelet donation and the second platelet donation to a pathogen inactivation process. In some embodiments, the method further comprises subjecting each of the first platelet donation, the second platelet donation and the third platelet donation to a pathogen inactivation process. In some embodiments, the method further comprises subjecting the pooled platelet preparation to a pathogen inactivation process. In some embodiments, subjecting the platelet donation or pooled platelet preparation to a pathogen inactivation process comprises treating the platelet donation or pooled platelet preparation with a pathogen inactivating compound. In some embodiments, the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540. In some embodiments, the pathogen inactivating compound is a psoralen. In some embodiments, the pathogen inactivating compound is amotosalen.

In some of the aforementioned embodiments, the platelet units (e.g., each of the platelet units) in the plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ platelets. In some embodiments, the platelet units each comprise at least about 3.0×10¹¹ platelets, and the pooled platelet preparation comprises at least about 6.0×10¹¹, at least about 6.5×10¹¹, at least about 7.0×10¹¹, at least about 7.5×10¹¹, at least about 8.0×10¹¹, at least about 8.5×10¹¹, at least about 9.0×10¹¹, at least about 9.5×10¹¹, at least about 10.0×10¹¹, at least about 10.5×10¹¹, at least about 11.0×10¹¹, at least about 11.5×10¹¹, at least about 12.0×10¹¹, at least about 12.5×10¹¹, at least about 13.0×10¹¹, at least about 13.5×10¹¹, at least about 14.0×10¹¹, at least about 14.5×10¹¹, or at least about 15.0×10¹¹ platelets. In some embodiments, the platelet units each comprise at least about 3.0×10¹¹ platelets, and the pooled platelet preparation comprises at least about 6.1×10¹¹, at least about 6.2×10¹¹, at least about 6.3×10¹¹, at least about 6.4×10¹¹, or at least about 6.5×10¹¹ platelets. In some embodiments, the platelet units each comprise at least about 3.0×10¹¹ platelets, and the pooled platelet preparation comprises at least about 9.2×10¹¹, at least about 9.3×10¹¹, at least about 9.4×10¹¹, at least about 9.5×10¹¹, or at least about 9.6×10¹¹ platelets. In some embodiments, the platelet units each comprise at least about 3.0×10¹¹ platelets, and the pooled platelet preparation comprises at least about 12.3×10¹¹, at least about 12.4×10¹¹, at least about 12.5×10¹¹, at least about 12.6×10¹¹, at least about 12.7×10¹¹ platelets. In some embodiments, the platelet units each comprise at least about 3.0×10¹¹ platelets, and the pooled platelet preparation comprises at least about 15.4×10¹¹, at least about 15.5×10¹¹, at least about 15.6×10¹¹, at least about 15.7×10¹¹, at least about 15.8×10¹¹, at least about 16.0×10¹¹ platelets. In some embodiments, the platelet units each comprise at least about 3.0×10¹¹ platelets, and the pooled platelet preparation comprises less than about 16.0×10¹¹, less than about 14.0×10¹¹, or less than about 12.0×10¹¹ platelets. In some embodiments, the platelet units each comprise at least about 3.0×10¹¹ platelets, and the pooled platelet preparation comprises about 6.0×10¹¹, about 6.5×10¹¹, about 7.0×10¹¹, about 7.5×10¹¹ about 8.0×10¹¹, about 8.5×10¹¹, about 9.0×10¹¹, about 9.5×10¹¹, about 10.0×10¹¹, about 10.5×10¹¹, about 11.0×10¹¹, about 11.5×10¹¹, about 12.0×10¹¹, about 12.5×10¹¹, about 13.0×10¹¹, about 13.5×10¹¹, about 14.0×10¹¹, about 14.5×10¹¹, or about 15.0×10¹¹ platelets.

In some embodiments, the platelet units each comprise at least about 2.0×10¹¹ platelets, and the pooled platelet preparation comprises at least about 5.0×10¹¹, at least about 5.5×10¹¹, at least about 6.0×10¹¹, at least about 6.5×10¹¹, at least about 7.0×10¹¹, at least about 7.5×10¹¹, at least about 8.0×10¹¹, at least about 8.5×10¹¹, at least about 9.0×10¹¹, at least about 9.5×10¹¹, at least about 10.0×10¹¹, at least about 10.5×10¹¹, at least about 11.0×10¹¹, at least about 11.5×10¹¹, at least about 12.0×10¹¹, at least about 12.5×10¹¹, at least about 13.0×10¹¹, at least about 13.5×10¹¹, at least about 14.0×10¹¹, at least about 14.5×10¹¹, or at least about 15.0×10¹¹ platelets. In some embodiments, the platelet units each comprise at least about 2.0×10¹¹ platelets, and the pooled platelet preparation comprises less than about 16.0×10¹¹, less than about 14.0×10¹¹, or less than about 12.0×10¹¹ platelets. In some embodiments, the platelet units each comprise at least about 2.0×10¹¹ platelets, and the pooled platelet preparation comprises about 5.0×10¹¹, about 5.5×10¹¹, about 6.0×10¹¹, about 6.5×10¹¹ about 7.0×10¹¹, about 7.5×10¹¹, about 8.0×10¹¹, about 8.5×10¹¹, about 9.0×10¹¹, about 9.5×10¹¹, about 10.0×10¹¹, about 10.5×10¹¹, about 11.0×10¹¹, about 11.5×10¹¹, about 12.0×10¹¹, about 12.5×10¹¹, about 13.0×10¹¹, about 13.5×10¹¹, about 14.0×10¹¹, about 14.5×10¹¹, or about 15.0×10¹¹ platelets.

In some embodiments, the platelet units each comprise at least about 2.2×10¹¹ platelets, and the pooled platelet preparation comprises at least about 5.0×10¹¹, at least about 5.5×10¹¹, at least about 6.0×10¹¹, at least about 6.5×10¹¹, at least about 7.0×10¹¹, at least about 7.5×10¹¹, at least about 8.0×10¹¹, at least about 8.5×10¹¹, at least about 9.0×10¹¹, at least about 9.5×10¹¹, at least about 10.0×10¹¹, at least about 10.5×10¹¹, at least about 11.0×10¹¹, at least about 11.5×10¹¹, at least about 12.0×10¹¹, at least about 12.5×10¹¹, at least about 13.0×10¹¹, at least about 13.5×10¹¹, at least about 14.0×10¹¹, at least about 14.5×10¹¹, or at least about 15.0×10¹¹ platelets. In some embodiments, the platelet units each comprise at least about 2.2×10¹¹ platelets, and the pooled platelet preparation comprises less than about 16.0×10¹¹, less than about 14.0×10¹¹, or less than about 12.0×10¹¹ platelets. In some embodiments, the platelet units each comprise at least about 2.2×10¹¹ platelets, and the pooled platelet preparation comprises about 5.0×10¹¹, about 5.5×10¹¹, about 6.0×10¹¹, about 6.5×10¹¹ about 7.0×10¹¹, about 7.5×10¹¹, about 8.0×10¹¹, about 8.5×10¹¹, about 9.0×10¹¹, about 9.5×10¹¹, about 10.0×10¹¹, about 10.5×10¹¹, about 11.0×10¹¹, about 11.5×10¹¹, about 12.0×10¹¹, about 12.5×10¹¹, about 13.0×10¹¹, about 13.5×10¹¹, about 14.0×10¹¹, about 14.5×10¹¹, or about 15.0×10¹¹ platelets.

In some embodiments, the platelet units each comprise at least about 2.4×10¹¹ platelets, and the pooled platelet preparation comprises at least about 5.0×10¹¹, at least about 5.5×10¹¹, at least about 6.0×10¹¹, at least about 6.5×10¹¹, at least about 7.0×10¹¹, at least about 7.5×10¹¹, at least about 8.0×10¹¹, at least about 8.5×10¹¹, at least about 9.0×10¹¹, at least about 9.5×10¹¹, at least about 10.0×10¹¹, at least about 10.5×10¹¹, at least about 11.0×10¹¹, at least about 11.5×10¹¹, at least about 12.0×10¹¹, at least about 12.5×10¹¹, at least about 13.0×10¹¹, at least about 13.5×10¹¹, at least about 14.0×10¹¹, at least about 14.5×10¹¹, or at least about 15.0×10¹¹ platelets. In some embodiments, the platelet units each comprise at least about 2.4×10¹¹ platelets, and the pooled platelet preparation comprises less than about 16.0×10¹¹, less than about 14.0×10¹¹, or less than about 12.0×10¹¹ platelets. In some embodiments, the platelet units each comprise at least about 2.4×10¹¹ platelets, and the pooled platelet preparation comprises about 5.0×10¹¹, about 5.5×10¹¹, about 6.0×10¹¹, about 6.5×10¹¹ about 7.0×10¹¹, about 7.5×10¹¹, about 8.0×10¹¹, about 8.5×10¹¹, about 9.0×10¹¹, about 9.5×10¹¹, about 10.0×10¹¹, about 10.5×10¹¹, about 11.0×10¹¹, about 11.5×10¹¹, about 12.0×10¹¹, about 12.5×10¹¹, about 13.0×10¹¹, about 13.5×10¹¹, about 14.0×10¹¹, about 14.5×10¹¹, or about 15.0×10¹¹ platelets.

In some embodiments, the platelet units each comprise at least about 2.6×10¹¹ platelets, and the pooled platelet preparation comprises at least about 5.0×10¹¹, at least about 5.5×10¹¹, at least about 6.0×10¹¹, at least about 6.5×10¹¹, at least about 7.0×10¹¹, at least about 7.5×10¹¹, at least about 8.0×10¹¹, at least about 8.5×10¹¹, at least about 9.0×10¹¹, at least about 9.5×10¹¹, at least about 10.0×10¹¹, at least about 10.5×10¹¹, at least about 11.0×10¹¹, at least about 11.5×10¹¹, at least about 12.0×10¹¹, at least about 12.5×10¹¹, at least about 13.0×10¹¹, at least about 13.5×10¹¹, at least about 14.0×10¹¹, at least about 14.5×10¹¹, or at least about 15.0×10¹¹ platelets. In some embodiments, the platelet units each comprise at least about 2.6×10¹¹ platelets, and the pooled platelet preparation comprises less than about 16.0×10¹¹, less than about 14.0×10¹¹, or less than about 12.0×10¹¹ platelets. In some embodiments, the platelet units each comprise at least about 2.6×10¹¹ platelets, and the pooled platelet preparation comprises about 5.0×10¹¹, about 5.5×10¹¹, about 6.0×10¹¹, about 6.5×10¹¹ about 7.0×10¹¹, about 7.5×10¹¹, about 8.0×10¹¹, about 8.5×10¹¹, about 9.0×10¹¹, about 9.5×10¹¹, about 10.0×10¹¹, about 10.5×10¹¹, about 11.0×10¹¹, about 11.5×10¹¹, about 12.0×10¹¹, about 12.5×10¹¹, about 13.0×10¹¹, about 13.5×10¹¹, about 14.0×10¹¹, about 14.5×10¹¹, or about 15.0×10¹¹ platelets.

In some embodiments, the platelet units each comprise at least about 2.8×10¹¹ platelets, and the pooled platelet preparation comprises at least about 5.0×10¹¹, at least about 5.5×10¹¹, at least about 6.0×10¹¹, at least about 6.5×10¹¹, at least about 7.0×10¹¹, at least about 7.5×10¹¹, at least about 8.0×10¹¹, at least about 8.5×10¹¹, at least about 9.0×10¹¹, at least about 9.5×10¹¹, at least about 10.0×10¹¹, at least about 10.5×10¹¹, at least about 11.0×10¹¹, at least about 11.5×10¹¹, at least about 12.0×10¹¹, at least about 12.5×10¹¹, at least about 13.0×10¹¹, at least about 13.5×10¹¹, at least about 14.0×10¹¹, at least about 14.5×10¹¹, or at least about 15.0×10¹¹ platelets. In some embodiments, the platelet units each comprise at least about 2.8×10¹¹ platelets, and the pooled platelet preparation comprises less than about 16.0×10¹¹, less than about 14.0×10¹¹, or less than about 12.0×10¹¹ platelets. In some embodiments, the platelet units each comprise at least about 2.8×10¹¹ platelets, and the pooled platelet preparation comprises about 5.0×10¹¹, about 5.5×10¹¹, about 6.0×10¹¹, about 6.5×10¹¹ about 7.0×10¹¹, about 7.5×10¹¹, about 8.0×10¹¹, about 8.5×10¹¹, about 9.0×10¹¹, about 9.5×10¹¹, about 10.0×10¹¹, about 10.5×10¹¹, about 11.0×10¹¹, about 11.5×10¹¹, about 12.0×10¹¹, about 12.5×10¹¹, about 13.0×10¹¹, about 13.5×10¹¹, about 14.0×10¹¹, about 14.5×10¹¹, or about 15.0×10¹¹ platelets.

In some of the aforementioned embodiments, the platelet units (e.g., each platelet unit) in the plurality of platelet units comprise a therapeutic dose (e.g., therapeutic dosage unit) of platelets suitable for infusion into a human subject (e.g., subject in need of a platelet infusion). In some embodiments, the therapeutic dose comprises a minimum number (e.g., at least a minimum number) of platelets as defined by criteria (e.g., acceptance criteria) of a governmental agency, regulatory agency, institution and/or accrediting organization (e.g., governmental agency, regulatory agency, institution and/or accrediting organization responsible for donated blood products (e.g., donated platelets)). In some embodiments, the platelet units are prepared in the country of the governmental agency, regulatory agency, institution and/or accrediting organization defining the criteria of a therapeutic dose of platelets. In some embodiments, at least about 75%, at least about 80%, at least about 85%, at least about 90%, at least about 95%, at least about 98%, at least about 99% or 100% of the platelet units in the plurality of platelet units comprise the minimum number of platelets of a therapeutic dose. In some embodiments, each of the platelet units in the plurality of platelet units comprises the minimum number of platelets of a therapeutic dose. In some embodiments, the minimum number of platelets in a therapeutic dose is at least about at least about 2.0×10¹¹ platelets, at least about 2.2×10¹¹ platelets, 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ platelets.

In some of the aforementioned embodiments, targeting the platelet collection comprises determining an optimal platelet collection. In some embodiments, an optimal platelet collection is a maximum platelet collection (e.g., maximum that can be safely collected, maximum according to regulatory and/or industry standards or regulations) that can be collected from a donor. In some embodiments, an optimal platelet collection is less than a maximum platelet collection that can be collected from a donor, but greater than the platelet collection required for a particular number (e.g., targeted number, desired number) of platelet units. In some embodiments, targeting the platelet collection comprises providing an input (e.g., programming) to an apheresis system. In some embodiments, targeting the platelet collection comprises providing a donation volume to an apheresis system. In some embodiments, targeting the platelet collection comprises providing a duration time for the platelet collection to an apheresis system. In some embodiments, targeting the platelet collection comprises providing a donation volume and/or duration time for the platelet collection that factors in (e.g., accounts for) variation (e.g., distribution) in actual platelet collection yields as compared to targeted collection yields. In some embodiments, the first platelet donation and the second platelet donation are combined in a single container of suitable size for the volume of the pooled platelet preparation. In some embodiments, the first platelet donation and the second platelet donation are of suitable volume for treating with a pathogen inactivating compound. In some embodiments, the first platelet donation and the second platelet donation are of suitable volume and platelet number (e.g., platelet quantity) for treating with a pathogen inactivating compound. In some embodiments, the pooled platelet preparation is of suitable volume for treating with a pathogen inactivating compound. In some embodiments, the pooled platelet preparation is of suitable volume and platelet number for treating with a pathogen inactivating compound.

In another aspect, the present disclosure provides a method of preparing a plurality of platelet units suitable for infusion (e.g., suitable for infusion into a subject), comprising: a) maintaining an inventory log of an inventory of apheresis-collected platelet preparations, the inventory log comprising for each platelet preparation a unique identifier, a donor blood type, a donation time (e.g., date, time, date and time) and a quantity (e.g., total platelet number, platelet count, platelet concentration and volume) of platelets in the preparation, b) identifying an available donor suitable for donating platelets by apheresis, c) selecting from the inventory log at least one (e.g., one or more) platelet preparation in the inventory for combining with platelets collected from the available donor, d) collecting a new platelet donation by apheresis from the available donor, e) combining the at least one selected platelet preparation and the new platelet donation to yield a pooled platelet preparation, and f) separating the pooled platelet preparation into a plurality of platelet units (e.g., therapeutic doses, therapeutic dosage units) each in an individual container, wherein the plurality of platelet units comprises a greater number of platelet units than the total number of platelet units that can be prepared from the at least one selected platelet preparation and the new platelet donation without combining the at least one selected platelet preparation and new platelet donation. In some embodiments, the inventory log further comprises for each platelet preparation a donor gender and/or a platelet preparation volume. In some embodiments, the inventory of apheresis-collected platelet preparations comprises existing platelet preparations (e.g., existing inventory) that have already been collected. In some embodiments, the inventory of apheresis-collected platelet preparations comprises platelet preparations being collected from a present donor (e.g., donor present at a blood center), wherein the present donor is not the available donor. In some embodiments, the platelet preparations being collected from a present donor comprise platelets being collected at about the same time (e.g., overlapping time) as the collecting a new platelet donation by apheresis from the available donor. In some embodiments, the platelet preparations being collected from a present donor comprise platelets that will be collected from a present donor. In some embodiments, the available donor is a new donor (e.g., donor that has not previously donated at a blood center, donor history is not available at a blood center). In some embodiments, the available donor is a previous donor (e.g., donor that has previously donated at a blood center, donor history is available at a blood center).

In some embodiments, the method further comprises determining for the available donor one or more donor parameters selected from the group consisting of gender, physical size (e.g., weight, height), hemoglobin level and platelet count (e.g., platelet count on day of donation, pre-donation platelet count). In some embodiments, the method further comprises determining a maximum amount (e.g., maximum safe amount, maximum volume, maximum number) of platelets that can be collected from the available donor by apheresis.

In some embodiments, the at least one selected platelet preparation comprises less than the number (e.g., less than the minimum number, less than the minimum quantity) of platelets required to prepare one platelet unit. In some embodiments, the at least one selected platelet preparation comprises an amount (e.g., number) of platelets that is less than about 99%, 98%, 95%, 90%, 85%, 80%, 75%, 70%, 65%, 60%, 55%, 50%, 45%, 40%, 35%, 30% or less than about 25% of the number of platelets required to prepare one platelet unit. In some embodiments, the at least one selected platelet preparation comprises an amount of platelets that is at least 10% of the number of platelets required to prepare one platelet unit. In some embodiments, the at least one selected platelet preparation comprises an amount of platelets that is about 10% to about 98%, about 15% to about 98%, about 20% to about 98%, about 25% to about 98%, about 30% to about 98%, about 35% to about 98%, about 40% to about 98% or about 50% to about 98% of the number of platelets required to prepare one platelet unit. In some embodiments, the at least one selected platelet preparation comprises an amount of platelets that is about 98%, 95%, 90%, 85%, 80%, 75%, 70%, 65%, 60%, 55%, 50%, 45%, 40%, 35%, 30% or about 25% of the number of platelets required to prepare one platelet unit.

In some embodiments, the at least one selected platelet preparation comprises greater than the number (e.g., greater than the minimum number, greater than the minimum quantity) of platelets required to prepare one platelet unit, but less than the number (e.g., less than the minimum number, less than the minimum quantity) of platelets required to prepare two platelet units. In some embodiments, greater than the number of platelets required to prepare one platelet unit, but less than the number of platelets required to prepare two platelet units is about 1.1-fold, 1.2-fold, 1.3-fold, 1.4-fold, 1.5-fold, 1.6-fold, 1.7-fold, 1.8-fold, 1.9-fold, 1.95-fold or about 1.98-fold the number of platelets required to prepare one platelet unit. In some embodiments, greater than the number of platelets required to prepare one platelet unit, but less than the number of platelets required to prepare two platelet units is about 1.1-fold to about 1.98-fold, about 1.15-fold to about 1.98-fold, about 1.2-fold to about 1.98-fold, about 1.25-fold to about 1.98-fold, about 1.3-fold to about 1.98-fold, about 1.35-fold to about 1.98-fold, about 1.4-fold to about 1.98-fold or about 1.5-fold to about 1.98-fold the number of platelets required to prepare one platelet unit. In some embodiments, greater than the number of platelets required to prepare one platelet unit, but less than the number of platelets required to prepare two platelet units is about 105%, 110%, 115%, 120%, 125%, 130%, 135%, 140%, 145%, 150%, 155%, 160%, 165%, 170%, 175%, 180%, 185%, 190% or about 195% the number of platelets required to prepare one platelet unit. In some embodiments, greater than the number of platelets required to prepare one platelet unit, but less than the number of platelets required to prepare two platelet units is about 110% to about 198%, about 115% to about 198%, about 120% to about 198%, about 125% to about 198%, about 130% to about 198%, about 135% to about 198%, about 140% to about 198% or about 150% to about 198% the number of platelets required to prepare one platelet unit.

In some embodiments, the at least one selected platelet preparation comprises greater than the number of platelets required to prepare two platelet units, but less than the number of platelets required to prepare three platelet units. In some embodiments, greater than the number of platelets required to prepare two platelet units, but less than the number of platelets required to prepare three platelet units is about 1.1-fold, 1.2-fold, 1.3-fold, 1.4-fold, 1.5-fold, 1.6-fold, 1.7-fold, 1.8-fold, 1.9-fold, 1.95-fold or about 1.98-fold the number of platelets required to prepare two platelet units. In some embodiments, greater than the number of platelets required to prepare two platelet units, but less than the number of platelets required to prepare three platelet units is about 1.1-fold to about 1.98-fold, about 1.15-fold to about 1.98-fold, about 1.2-fold to about 1.98-fold, about 1.25-fold to about 1.98-fold, about 1.3-fold to about 1.98-fold, about 1.35-fold to about 1.98-fold, about 1.4-fold to about 1.98-fold or about 1.5-fold to about 1.98-fold the number of platelets required to prepare two platelet units. In some embodiments, greater than the number of platelets required to prepare two platelet units, but less than the number of platelets required to prepare three platelet units is about 105%, 110%, 115%, 120%, 125%, 130%, 135%, 140%, 145%, 150%, 155%, 160%, 165%, 170%, 175%, 180%, 185%, 190% or about 195% the number of platelets required to prepare two platelet units. In some embodiments, greater than the number of platelets required to prepare two platelet units, but less than the number of platelets required to prepare three platelet units is about 110% to about 198%, about 115% to about 198%, about 120% to about 198%, about 125% to about 198%, about 130% to about 198%, about 135% to about 198%, about 140% to about 198% or about 150% to about 198% the number of platelets required to prepare two platelet units. In some embodiments, the at least one selected platelet preparation comprises at least about 225%, 230%, 235%, 240%, 245%, 250%, 255%, 260%, 265%, 270%, 275%, 280%, 285%, 290% or about 295% of the number of platelets required to prepare one platelet unit, but less than the number of platelets required to prepare three platelet units. In some embodiments, the at least one selected platelet preparation comprises about 225% to about 298%, about 230% to about 298%, about 235% to about 298%, about 240% to about 298%, about 245% to about 298%, or about 250% to about 298% of the number of platelets required to prepare one platelet unit.

In some embodiments, the at least one selected platelet preparation comprises greater than the number of platelets required to prepare three platelet units, but less than the number of platelets required to prepare four platelet units. In some embodiments, greater than the number of platelets required to prepare three platelet units, but less than the number of platelets required to prepare four platelet units is about 1.1-fold, 1.2-fold, 1.3-fold, 1.4-fold, 1.5-fold, 1.6-fold, 1.7-fold, 1.8-fold, 1.9-fold, 1.95-fold or about 1.98-fold the number of platelets required to prepare three platelet units. In some embodiments, greater than the number of platelets required to prepare three platelet units, but less than the number of platelets required to prepare four platelet units is about 1.1-fold to about 1.98-fold, about 1.15-fold to about 1.98-fold, about 1.2-fold to about 1.98-fold, about 1.25-fold to about 1.98-fold, about 1.3-fold to about 1.98-fold, about 1.35-fold to about 1.98-fold, about 1.4-fold to about 1.98-fold or about 1.5-fold to about 1.98-fold the number of platelets required to prepare three platelet units. In some embodiments, greater than the number of platelets required to prepare three platelet units, but less than the number of platelets required to prepare four platelet units is about 105%, 110%, 115%, 120%, 125%, 130%, 135%, 140%, 145%, 150%, 155%, 160%, 165%, 170%, 175%, 180%, 185%, 190% or about 195% the number of platelets required to prepare three platelet units. In some embodiments, greater than the number of platelets required to prepare three platelet units, but less than the number of platelets required to prepare four platelet units is about 110% to about 198%, about 115% to about 198%, about 120% to about 198%, about 125% to about 198%, about 130% to about 198%, about 135% to about 198%, about 140% to about 198% or about 150% to about 198% the number of platelets required to prepare three platelet units. In some embodiments, the at least one selected platelet preparation comprises at least about 325%, 330%, 335%, 340%, 345%, 350%, 355%, 360%, 365%, 370%, 375%, 380%, 385%, 390% or about 395% of the number of platelets required to prepare one platelet unit, but less than the number of platelets required to prepare four platelet units. In some embodiments, the at least one selected platelet preparation comprises about 325% to about 398%, about 330% to about 398%, about 335% to about 398%, about 340% to about 398%, about 345% to about 398%, or about 350% to about 398% of the number of platelets required to prepare one platelet unit

In some embodiments, the new platelet donation comprises less than the number (e.g., less than the minimum number, less than the minimum quantity) of platelets required to prepare one platelet unit. In some embodiments, the new platelet donation comprises an amount (e.g., number) of platelets that is less than about 99%, 98%, 95%, 90%, 85%, 80%, 75%, 70%, 65%, 60%, 55%, 50%, 45%, 40%, 35%, 30% or less than about 25% of the number of platelets required to prepare one platelet unit. In some embodiments, the new platelet donation comprises an amount of platelets that is at least 10% of the number of platelets required to prepare one platelet unit. In some embodiments, the new platelet donation comprises an amount of platelets that is about 10% to about 98%, about 15% to about 98%, about 20% to about 98%, about 25% to about 98%, about 30% to about 98%, about 35% to about 98%, about 40% to about 98% or about 50% to about 98% of the number of platelets required to prepare one platelet unit. In some embodiments, the new platelet donation comprises an amount of platelets that is about 98%, 95%, 90%, 85%, 80%, 75%, 70%, 65%, 60%, 55%, 50%, 45%, 40%, 35%, 30% or about 25% of the number of platelets required to prepare one platelet unit. In some embodiments, collecting the new platelet donation comprises targeting the platelet collection to yield a donation comprising less than the number of platelets required to prepare one platelet unit. In some embodiments, collecting the new platelet donation comprises targeting the platelet collection to yield a donation comprising less than about 99%, 98%, 95%, 90%, 85%, 80%, 75%, 70%, 65%, 60%, 55%, 50%, 45%, 40%, 35%, 30% or less than about 25% number of platelets required to prepare one platelet unit. In some embodiments, collecting the new platelet donation comprises targeting the platelet collection to yield a donation comprising about 10% to about 98%, about 15% to about 98%, about 20% to about 98%, about 25% to about 98%, about 30% to about 98%, about 35% to about 98%, about 40% to about 98% or about 50% to about 98% of the number of platelets required to prepare one platelet unit. In some embodiments, collecting the new platelet donation from the available donor comprises targeting the platelet collection to yield a donation comprising a sufficient number of platelets to prepare one, to prepare two, to prepare three, or to prepare four platelet units after combining the at least one selected platelet preparation and the new platelet donation and separating the resulting pooled platelet preparation into a plurality of platelet units. In some embodiments, the new platelet donation comprises a sufficient number of platelets that, when combined with the at least one selected platelet preparation, yields a pooled platelet preparation comprising a sufficient number of platelets to separate into one, into two, into three, or into four platelet units. In some embodiments, collecting the new platelet donation from the available donor comprises collecting a sufficient number of platelets to prepare one, to prepare two, to prepare three, or to prepare four platelet units from the pooled preparation.

In some embodiments, the new platelet donation comprises greater than the number of platelets required to prepare one platelet unit, but less than the number of platelets required to prepare two platelet units. In some embodiments, greater than the number of platelets required to prepare one platelet unit, but less than the number of platelets required to prepare two platelet units is about 1.1-fold, 1.2-fold, 1.3-fold, 1.4-fold, 1.5-fold, 1.6-fold, 1.7-fold, 1.8-fold, 1.9-fold, 1.95-fold or about 1.98-fold the number of platelets required to prepare one platelet unit. In some embodiments, greater than the number of platelets required to prepare one platelet unit, but less than the number of platelets required to prepare two platelet units is about 1.1-fold to about 1.98-fold, about 1.15-fold to about 1.98-fold, about 1.2-fold to about 1.98-fold, about 1.25-fold to about 1.98-fold, about 1.3-fold to about 1.98-fold, about 1.35-fold to about 1.98-fold, about 1.4-fold to about 1.98-fold or about 1.5-fold to about 1.98-fold the number of platelets required to prepare one platelet unit. In some embodiments, greater than the number of platelets required to prepare one platelet unit, but less than the number of platelets required to prepare two platelet units is about 105%, 110%, 115%, 120%, 125%, 130%, 135%, 140%, 145%, 150%, 155%, 160%, 165%, 170%, 175%, 180%, 185%, 190% or about 195% the number of platelets required to prepare one platelet unit. In some embodiments, greater than the number of platelets required to prepare one platelet unit, but less than the number of platelets required to prepare two platelet units is about 110% to about 198%, about 115% to about 198%, about 120% to about 198%, about 125% to about 198%, about 130% to about 198%, about 135% to about 198%, about 140% to about 198% or about 150% to about 198% the number of platelets required to prepare one platelet unit. In some embodiments, collecting the new platelet donation comprises targeting the platelet collection to yield a donation comprising at least about 125%, 130%, 135%, 140%, 145%, 150%, 155%, 160%, 165%, 170%, 175%, 180%, 185%, 190% or about 195% of the number of platelets required to prepare one platelet unit, but less than the number of platelets required to prepare two platelet units. In some embodiments, collecting the new platelet donation comprises targeting the platelet collection to yield a donation comprising about 110% to about 198%, about 115% to about 198%, about 120% to about 198%, about 125% to about 198%, about 130% to about 198%, about 135% to about 198%, about 140% to about 198%, about 145% to about 198%, or about 150% to about 198% the number of platelets required to prepare one platelet unit. In some embodiments, collecting the new platelet donation from the available donor comprises targeting the platelet collection to yield a donation comprising a sufficient number of platelets to prepare two, to prepare three, to prepare four, or to prepare five platelet units after combining the at least one selected platelet preparation and the new platelet donation and separating the resulting pooled platelet preparation into a plurality of platelet units. In some embodiments, the new platelet donation comprises a sufficient number of platelets that, when combined with the at least one selected platelet preparation, yields a pooled platelet preparation comprising a sufficient number of platelets to separate into two, into three, into four, or into five platelet units. In some embodiments, collecting the new platelet donation from the available donor comprises collecting a sufficient number of platelets to prepare two, to prepare three, to prepare four, or to prepare five platelet units from the pooled preparation.

In some embodiments, the new platelet donation comprises greater than the number of platelets required to prepare two platelet units, but less than the number of platelets required to prepare three platelet units. In some embodiments, greater than the number of platelets required to prepare two platelet units, but less than the number of platelets required to prepare three platelet units is about 1.1-fold, 1.2-fold, 1.3-fold, 1.4-fold, 1.5-fold, 1.6-fold, 1.7-fold, 1.8-fold, 1.9-fold, 1.95-fold or about 1.98-fold the number of platelets required to prepare two platelet units. In some embodiments, greater than the number of platelets required to prepare two platelet units, but less than the number of platelets required to prepare three platelet units is about 1.1-fold to about 1.98-fold, about 1.15-fold to about 1.98-fold, about 1.2-fold to about 1.98-fold, about 1.25-fold to about 1.98-fold, about 1.3-fold to about 1.98-fold, about 1.35-fold to about 1.98-fold, about 1.4-fold to about 1.98-fold or about 1.5-fold to about 1.98-fold the number of platelets required to prepare two platelet units. In some embodiments, greater than the number of platelets required to prepare two platelet units, but less than the number of platelets required to prepare three platelet units is about 105%, 110%, 115%, 120%, 125%, 130%, 135%, 140%, 145%, 150%, 155%, 160%, 165%, 170%, 175%, 180%, 185%, 190% or about 195% the number of platelets required to prepare two platelet units. In some embodiments, greater than the number of platelets required to prepare two platelet units, but less than the number of platelets required to prepare three platelet units is about 110% to about 198%, about 115% to about 198%, about 120% to about 198%, about 125% to about 198%, about 130% to about 198%, about 135% to about 198%, about 140% to about 198% or about 150% to about 198% the number of platelets required to prepare two platelet units. In some embodiments, the new platelet donation comprises at least about 225%, 230%, 235%, 240%, 245%, 250%, 255%, 260%, 265%, 270%, 275%, 280%, 285%, 290% or about 295% of the number of platelets required to prepare one platelet unit, but less than the number of platelets required to prepare three platelet units. In some embodiments, the new platelet donation comprises about 210% to about 298%, about 215% to about 298%, about 220% to about 298%, about 225% to about 298%, about 230% to about 298%, about 235% to about 298%, about 240% to about 298%, about 245% to about 298%, or about 250% to about 298% of the number of platelets required to prepare one platelet unit. In some embodiments, collecting the new platelet donation comprises targeting the platelet collection to yield a donation comprising at least about 225%, 230%, 235%, 240%, 245%, 250%, 255%, 260%, 265%, 270%, 275%, 280%, 285%, 290% or about 295% of the number of platelets required to prepare one platelet unit, but less than the number of platelets required to prepare three platelet units. In some embodiments, collecting the new platelet donation comprises targeting the platelet collection to yield a donation comprising about 210% to about 298%, about 215% to about 298%, about 220% to about 298%, about 225% to about 298%, about 230% to about 298%, about 235% to about 298%, about 240% to about 298%, about 245% to about 298%, or about 250% to about 298% the number of platelets required to prepare one platelet unit. In some embodiments, collecting the new platelet donation from the available donor comprises targeting the platelet collection to yield a donation comprising a sufficient number of platelets to prepare three, to prepare four, to prepare five, or to prepare six platelet units after combining the at least one selected platelet preparation and the new platelet donation and separating the resulting pooled platelet preparation into a plurality of platelet units. In some embodiments, the new platelet donation comprises a sufficient number of platelets that, when combined with the at least one selected platelet preparation, yields a pooled platelet preparation comprising a sufficient number of platelets to separate into three, into four, into five, or into six platelet units. In some embodiments, collecting the new platelet donation from the available donor comprises collecting a sufficient number of platelets to prepare three, to prepare four, to prepare five, or to prepare six platelet units from the pooled preparation.

In some embodiments, the new platelet donation comprises greater than the number of platelets required to prepare three platelet units, but less than the number of platelets required to prepare four platelet units. In some embodiments, greater than the number of platelets required to prepare three platelet units, but less than the number of platelets required to prepare four platelet units is about 1.1-fold, 1.2-fold, 1.3-fold, 1.4-fold, 1.5-fold, 1.6-fold, 1.7-fold, 1.8-fold, 1.9-fold, 1.95-fold or about 1.98-fold the number of platelets required to prepare three platelet units. In some embodiments, greater than the number of platelets required to prepare three platelet units, but less than the number of platelets required to prepare four platelet units is about 1.1-fold to about 1.98-fold, about 1.15-fold to about 1.98-fold, about 1.2-fold to about 1.98-fold, about 1.25-fold to about 1.98-fold, about 1.3-fold to about 1.98-fold, about 1.35-fold to about 1.98-fold, about 1.4-fold to about 1.98-fold or about 1.5-fold to about 1.98-fold the number of platelets required to prepare three platelet units. In some embodiments, greater than the number of platelets required to prepare three platelet units, but less than the number of platelets required to prepare four platelet units is about 105%, 110%, 115%, 120%, 125%, 130%, 135%, 140%, 145%, 150%, 155%, 160%, 165%, 170%, 175%, 180%, 185%, 190% or about 195% the number of platelets required to prepare three platelet units. In some embodiments, greater than the number of platelets required to prepare three platelet units, but less than the number of platelets required to prepare four platelet units is about 110% to about 198%, about 115% to about 198%, about 120% to about 198%, about 125% to about 198%, about 130% to about 198%, about 135% to about 198%, about 140% to about 198% or about 150% to about 198% the number of platelets required to prepare three platelet units. In some embodiments, the new platelet donation comprises at least about 325%, 330%, 335%, 340%, 345%, 350%, 355%, 360%, 365%, 370%, 375%, 380%, 385%, 390% or about 395% of the number of platelets required to prepare one platelet unit, but less than the number of platelets required to prepare three platelet units. In some embodiments, the new platelet donation comprises about 310% to about 398%, about 315% to about 398%, about 320% to about 398%, about 325% to about 398%, about 330% to about 398%, about 335% to about 398%, about 340% to about 398%, about 345% to about 398%, or about 350% to about 398% of the number of platelets required to prepare one platelet unit. In some embodiments, collecting the new platelet donation comprises targeting the platelet collection to yield a donation comprising at least about 325%, 330%, 335%, 340%, 345%, 350%, 355%, 360%, 365%, 370%, 375%, 380%, 385%, 390% or about 395% of the number of platelets required to prepare one platelet unit, but less than the number of platelets required to prepare three platelet units. In some embodiments, collecting the new platelet donation comprises targeting the platelet collection to yield a donation comprising about 310% to about 398%, about 315% to about 398%, about 320% to about 398%, about 325% to about 398%, about 330% to about 398%, about 335% to about 398%, about 340% to about 398%, about 345% to about 398%, or about 350% to about 398% the number of platelets required to prepare one platelet unit. In some embodiments, collecting the new platelet donation from the available donor comprises targeting the platelet collection to yield a donation comprising a sufficient number of platelets to prepare four, to prepare five, to prepare six, or to prepare seven platelet units after combining the at least one selected platelet preparation and the new platelet donation and separating the resulting pooled platelet preparation into a plurality of platelet units. In some embodiments, the new platelet donation comprises a sufficient number of platelets that, when combined with the at least one selected platelet preparation, yields a pooled platelet preparation comprising a sufficient number of platelets to separate into four, into five, into six, or into seven platelet units. In some embodiments, collecting the new platelet donation from the available donor comprises collecting a sufficient number of platelets to prepare four, to prepare five, to prepare six, or to prepare seven platelet units from the pooled preparation.

In some embodiments, the at least one selected platelet preparation comprises less than the number of platelets required to prepare one platelet unit and the new platelet donation comprises greater than the number of platelets required to prepare one platelet unit, but less than the number of platelets required to prepare two platelet units. In some embodiments, the at least one selected platelet preparation comprises less than the number of platelets required to prepare one platelet unit and the new platelet donation comprises greater than the number of platelets required to prepare two platelet units, but less than the number of platelets required to prepare three platelet units. In some embodiments, the at least one selected platelet preparation comprises less than the number of platelets required to prepare one platelet unit and the new platelet donation comprises greater than the number of platelets required to prepare three platelet units, but less than the number of platelets required to prepare four platelet units. In some embodiments, the pooled platelet preparation comprises a sufficient number of platelets to prepare two, to prepare three, or to prepare four platelet units. In some embodiments, the plurality of platelet units comprises two platelet units, three platelet units, or four platelet units.

In some embodiments, the new platelet donation comprises less than the number of platelets required to prepare one platelet unit and the at least one selected platelet preparation comprises greater than the number of platelets required to prepare one platelet unit, but less than the number of platelets required to prepare two platelet units. In some embodiments, the new platelet donation comprises less than the number of platelets required to prepare one platelet unit and the at least one selected platelet preparation comprises greater than the number of platelets required to prepare two platelet units, but less than the number of platelets required to prepare three platelet units. In some embodiments, the new platelet donation comprises less than the number of platelets required to prepare one platelet unit and the at least one selected platelet preparation comprises greater than the number of platelets required to prepare three platelet units, but less than the number of platelets required to prepare four platelet units. In some embodiments, the pooled platelet preparation comprises a sufficient number of platelets to prepare two, to prepare three, or to prepare four platelet units. In some embodiments, the plurality of platelet units comprises two platelet units, three platelet units, or four platelet units.

In some embodiments, the at least one selected platelet preparation comprises greater than the number of platelets required to prepare one platelet unit, but less than the number of platelets required to prepare two platelet units, and the new platelet donation comprises greater than the number of platelets required to prepare one platelet unit, but less than the number of platelets required to prepare two platelet units. In some embodiments, the at least one selected platelet preparation comprises greater than the number of platelets required to prepare one platelet unit, but less than the number of platelets required to prepare two platelet units, and the new platelet donation comprises greater than the number of platelets required to prepare two platelet units, but less than the number of platelets required to prepare three platelet units. In some embodiments, the at least one selected platelet preparation comprises greater than the number of platelets required to prepare one platelet unit, but less than the number of platelets required to prepare two platelet units, and the new platelet donation comprises greater than the number of platelets required to prepare three platelet units, but less than the number of platelets required to prepare four platelet units. In some embodiments, the pooled platelet preparation comprises a sufficient number of platelets to prepare three, to prepare four, or to prepare five platelet units. In some embodiments, the plurality of platelet units comprises three platelet units, four platelet units, or five platelet units.

In some embodiments, the at least one selected platelet preparation comprises greater than the number of platelets required to prepare two platelet units, but less than the number of platelets required to prepare three platelet units, and the new platelet donation comprises greater than the number of platelets required to prepare two platelet units, but less than the number of platelets required to prepare three platelet units. In some embodiments, the at least one selected platelet preparation comprises greater than the number of platelets required to prepare two platelet units, but less than the number of platelets required to prepare three platelet units, and the new platelet donation comprises greater than the number of platelets required to prepare three platelet units, but less than the number of platelets required to prepare four platelet units. In some embodiments, the at least one selected platelet preparation comprises greater than the number of platelets required to prepare three platelet units, but less than the number of platelets required to prepare four platelet units, and the new platelet donation comprises greater than the number of platelets required to prepare three platelet units, but less than the number of platelets required to prepare four platelet units. In some embodiments, the pooled platelet preparation comprises a sufficient number of platelets to prepare five, to prepare six, or to prepare seven platelet units. In some embodiments, the plurality of platelet units comprises five platelet units, six platelet units, or seven platelet units.

In some embodiments, the method further comprises identifying a second available donor suitable for donating platelets by apheresis, collecting a second new platelet donation by apheresis from the second available donor, combining the second new platelet donation with the at least one selected platelet preparation and the new platelet donation to yield the pooled platelet preparation, and separating the pooled platelet preparation into a plurality of platelet units each in an individual container, wherein the plurality of platelet units comprises a greater number of platelet units than the total number of platelet units that can be prepared from the at least one selected platelet preparation, the new platelet donation and the second new platelet donation without combining the at least one selected platelet preparation, the new platelet donation and the second new platelet donation. In some embodiments, the method further comprises subjecting the at least one selected platelet preparation, the new platelet donation, and/or the second new platelet donation to a pathogen inactivation process. In some embodiments, the method further comprises subjecting each of the at least one selected platelet preparation and the new platelet donation a pathogen inactivation process. In some embodiments, the method further comprises subjecting each of the at least one selected platelet preparation, the new platelet donation and the second new platelet donation a pathogen inactivation process. In some embodiments, the method further comprises subjecting the pooled platelet preparation a pathogen inactivation process. In some embodiments, subjecting the at least one selected platelet preparation, new platelet donation, second new platelet donation and/or pooled platelet preparation to a pathogen inactivation process comprises treating the at least one selected platelet preparation, new platelet donation, second new platelet donation and/or pooled platelet preparation with a pathogen inactivating compound. In some embodiments, the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540. In some embodiments, the pathogen inactivating compound is a psoralen. In some embodiments, the pathogen inactivating compound is amotosalen.

In some embodiments, the method further comprises collecting a third platelet donation by apheresis from a third donor, combining the third platelet donation with the at least one selected platelet preparation and the new platelet donation to yield a pooled platelet preparation, and separating the pooled platelet preparation into a plurality of platelet units each in an individual container, wherein the plurality of platelet units comprises a greater number of platelet units than the total number of platelet units that can be prepared from the at least one selected platelet preparation, the new platelet donation and the third platelet donation without combining the at least one selected platelet preparation, the new platelet donation and the third platelet donation. In some embodiments, the method further comprises subjecting the at least one selected platelet preparation, the new platelet donation, and/or the third platelet donation to a pathogen inactivation process. In some embodiments, the method further comprises subjecting each of the at least one selected platelet preparation and the new platelet donation a pathogen inactivation process. In some embodiments, the method further comprises subjecting each of the at least one selected platelet preparation, the new platelet donation and the third platelet donation a pathogen inactivation process. In some embodiments, the method further comprises subjecting the pooled platelet preparation a pathogen inactivation process. In some embodiments, subjecting the at least one selected platelet preparation, new platelet donation, third platelet donation and/or pooled platelet preparation to a pathogen inactivation process comprises treating the at least one selected platelet preparation, new platelet donation, third platelet donation and/or pooled platelet preparation with a pathogen inactivating compound. In some embodiments, the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540. In some embodiments, the pathogen inactivating compound is a psoralen. In some embodiments, the pathogen inactivating compound is amotosalen.

In some of the aforementioned embodiments, the new platelet donation is collected less than about (e.g., within) 24 hours, 22 hours, 20 hours, 16 hours, 12 hours, 10 hours, 8 hours, 6 hours, 5 hours, 4 hours, 3 hours, 2 hours or less than about 1 hour from the time the at least one selected platelet preparation is collected. In some embodiments, the new platelet donation is collected within about 7 days, within about 6 days, within about 5 days, within about 4 days, within about 3 days, within about 2 days, or within about 1 day from the time the at least one selected platelet preparation is collected. In some embodiments, the new platelet donation is collected about the same time (e.g., overlapping collection time) as the at least one selected platelet preparation. In some embodiments, the new platelet donation is collected within 20 hours from the time the at least one selected platelet preparation is collected. In some embodiments, the new platelet donation is collected within 12 hours from the time the at least one selected platelet preparation is collected. In some embodiments, the new platelet donation is collected within 6 hours from the time the at least one selected platelet preparation is collected. In some of the aforementioned embodiments, the second new platelet donation (or third platelet donation) is collected less than about (e.g., within) 24 hours, 22 hours, 20 hours, 16 hours, 12 hours, 10 hours, 8 hours, 6 hours, 5 hours, 4 hours, 3 hours, 2 hours or less than about 1 hour from the time the at least one selected platelet preparation is collected. In some embodiments, the second new platelet donation is collected within about 7 days, within about 6 days, within about 5 days, within about 4 days, within about 3 days, within about 2 days, or within about 1 day from the time the at least one selected platelet preparation is collected. In some embodiments, the second new platelet donation (or third platelet donation) is collected about the same time (e.g., overlapping collection time) as the at least one selected platelet preparation. In some embodiments, the second new platelet donation (third platelet donation) is collected within 20 hours from the time the at least one selected platelet preparation is collected. In some embodiments, the second new platelet donation (or third platelet donation) is collected within 12 hours from the time the at least one selected platelet preparation is collected. In some embodiments, the second new platelet donation (or third platelet donation) is collected within 6 hours from the time the at least one selected platelet preparation is collected. In some embodiments, the second new platelet donation is collected within about 24 hours, 22 hours, 20 hours, 16 hours, 12 hours, 10 hours, 8 hours, 6 hours, 5 hours, 4 hours, 3 hours, 2 hours or about 1 hour from the time the at least one selected platelet preparation and the new platelet donation are collected. In some embodiments, the at least one selected platelet preparation, the new platelet donation and the second new platelet donation (or third platelet donation) are collected within a period (e.g., the same period) of about 24 hours, 22 hours, 20 hours, 16 hours, 12 hours, 10 hours, 8 hours, 6 hours, 5 hours, 4 hours, 3 hours, 2 hours or about 1 hour. In some embodiments, the at least one selected platelet preparation, the new platelet donation and the second new platelet donation (or third platelet donation) are collected within about 7 days, within about 6 days, within about 5 days, within about 4 days, within about 3 days, within about 2 days, within about 1 day, within about 72 hours, within about 48 hours, within about 36 hours, within about 24 hours, within about 20 hours, within about 12 hours or within about 6 hours of each other. In some embodiments, the at least one selected platelet preparation, the new platelet donation and the second new platelet donation (or third platelet donation) are collected at about the same time (e.g., overlapping collection times).

In some of the aforementioned embodiments, the pooled platelet preparation comprises platelets (e.g., the at least one selected platelet preparation, the new platelet donation, the second new platelet donation, the third platelet donation) only from donors of the same ABO blood type.

In some of the aforementioned embodiments, the platelet units (e.g., each of the platelet units) in the plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ platelets. In some embodiments, the platelet units each comprise at least about 3.0×10¹¹ platelets, and the pooled platelet preparation comprises at least about 6.2×10¹¹ platelets. In some embodiments, the platelet units each comprise at least about 3.0×10¹¹ platelets, and the pooled platelet preparation comprises at least about 9.3×10¹¹ platelets. In some embodiments, the platelet units each comprise at least about 3.0×10¹¹ platelets, and the pooled platelet preparation comprises at least about 12.4×10¹¹ platelets. In some embodiments, the platelet units each comprise at least about 3.0×10¹¹ platelets, and the pooled platelet preparation comprises at least about 15.5×10¹¹ platelets.

In some of the aforementioned embodiments, the platelet units (e.g., each platelet unit) in the plurality of platelet units comprise a therapeutic dose (e.g., therapeutic dosage unit) of platelets suitable for infusion into a human subject (e.g., subject in need of a platelet infusion). In some embodiments, the therapeutic dose comprises a minimum number (e.g., at least a minimum number) of platelets as defined by criteria (e.g., acceptance criteria) of a governmental agency, regulatory agency, institution and/or accrediting organization (e.g., governmental agency, regulatory agency, institution and/or accrediting organization for donated blood products (e.g., donated platelets)). In some embodiments, the platelet units are prepared in the country of the governmental agency, regulatory agency, institution and/or accrediting organization defining the criteria of a therapeutic dose of platelets. In some embodiments, at least about 75%, at least about 80%, at least about 85%, at least about 90%, at least about 95%, at least about 98%, at least about 99% or more of the platelet units in the plurality of platelet units comprise the minimum number of platelets of a therapeutic dose. In some embodiments, each of the platelet units in the plurality of platelet units comprises the minimum number of platelets of a therapeutic dose. In some embodiments, the minimum number of platelets in a therapeutic dose is at least about 2.0×10¹¹ platelets, at least about 2.2×10¹¹, platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ platelets.

In some of the aforementioned embodiments, targeting the platelet collection comprises determining an optimal platelet collection. In some embodiments, an optimal platelet collection is a maximum platelet collection (e.g., maximum that can be safely collected, maximum according to regulatory and/or industry standards or regulations) that can be collected from a donor. In some embodiments, targeting the platelet collection comprises providing an input (e.g., programming) to an apheresis system. In some embodiments, targeting the platelet collection comprises providing a donation volume to an apheresis system. In some embodiments, targeting the platelet collection comprises providing a duration time for the platelet collection to an apheresis system. In some embodiments, targeting the platelet collection comprises providing a donation volume and/or duration time for the platelet collection that factors in (e.g., accounts for) variation (e.g., distribution) in actual platelet collection yields as compared to targeted collection yields. In some embodiments, the at least one selected platelet preparation and the new platelet donation are combined in a single container of suitable size for the volume of the pooled platelet preparation. In some embodiments, the at least one selected platelet preparation and the new platelet donation are of suitable volume for treating with a pathogen inactivating compound. In some embodiments, the at least one selected platelet preparation and the new platelet donation are of suitable volume and platelet number (e.g., platelet quantity) for treating with a pathogen inactivating compound. In some embodiments, the pooled platelet preparation is of suitable volume for treating with a pathogen inactivating compound. In some embodiments, the pooled platelet preparation is of suitable volume and platelet number for treating with a pathogen inactivating compound.

In another aspect, the present disclosure provides a platelet unit suitable for infusion prepared by any of the aforementioned methods.

In another aspect, the present disclosure provides a method of preparing two or more platelet units (e.g., therapeutic dosage units of platelets) comprising: a) combining two or more apheresis-derived platelet donor units to form a pooled platelet composition, b) subjecting the pooled platelet composition to a pathogen inactivation process, and c) separating the pooled platelet composition into two or more platelet units (e.g., therapeutic dosage units of platelets), each in an individual container. In some embodiments, the method is a method of preparing three or more, four or more, five or more, six or more, or seven or more platelet units. In some embodiments, the disclosure provides a method of preparing two or more therapeutic dosage units of platelets (e.g., therapeutic dosage units of platelets suitable for infusion into a subject) comprising: a) combining two or more apheresis-derived platelet donor units to form a pooled platelet composition, b) subjecting the pooled platelet composition to a pathogen inactivation process, and c) separating the pooled platelet composition into two or more therapeutic dosage units of platelets, each in an individual container. In some embodiments, the method is a method of preparing three or more, four or more, five or more, six or more, or seven or more therapeutic dosage units of platelets. In some embodiments, the number of therapeutic dosage units of platelets following step c) is greater than the number of therapeutic dosage units of platelets that can be prepared from the two or more apheresis-derived platelet donor units individually, without combining the two or more donor units.

In some embodiments, each apheresis-derived platelet donor unit comprises greater than the number of platelets required to prepare one therapeutic dosage unit of platelets, but less than the number of platelets required to prepare two therapeutic dosage units of platelets. In some embodiments, each apheresis-derived platelet donor unit comprises greater than the number of platelets required to prepare two therapeutic dosage units of platelets, but less than the number of platelets required to prepare three therapeutic dosage units of platelets. In some embodiments, each apheresis-derived platelet donor unit comprises greater than the number of platelets required to prepare three therapeutic dosage units of platelets, but less than the number of platelets required to prepare four therapeutic dosage units of platelets.

In some embodiments, two apheresis-derived platelet donor units are combined and each donor unit comprises greater than the number of platelets required to prepare one therapeutic dosage unit of platelets, but less than the number of platelets required to prepare two therapeutic dosage units of platelets. In some embodiments, each donor unit comprises at least about 125%, at least about 130%, at least about 135%, at least about 140%, at least about 145%, or at least about at least about 150% of the number of platelets required to prepare one platelet unit. In some embodiments, each donor unit comprises less than about 198% of the number of platelets required to prepare one platelet unit. In some embodiments, two apheresis-derived platelet donor units are combined and each donor unit comprises greater than the number of platelets required to prepare two therapeutic dosage units of platelets, but less than the number of platelets required to prepare three therapeutic dosage units of platelets. In some embodiments, each donor unit comprises at least about 225%, at least about 230%, at least about 235%, at least about 240%, at least about 245%, or at least about at least about 250% of the number of platelets required to prepare one platelet unit. In some embodiments, each donor unit comprises less than about 298% of the number of platelets required to prepare one platelet unit. In some embodiments, two apheresis-derived platelet donor units are combined and each donor unit comprises greater than the number of platelets required to prepare three therapeutic dosage units of platelets, but less than the number of platelets required to prepare four therapeutic dosage units of platelets. In some embodiments, each donor unit comprises at least about 325%, at least about 330%, at least about 335%, at least about 340%, at least about 345%, or at least about at least about 350% of the number of platelets required to prepare one platelet unit. In some embodiments, each donor unit comprises less than about 398% of the number of platelets required to prepare one platelet unit. In some embodiments, two apheresis-derived platelet donor units are combined, and one of the two donor units (e.g., a first donor unit) comprises greater than the number of platelets required to prepare one therapeutic dosage unit of platelets, but less than the number of platelets required to prepare two therapeutic dosage units of platelets, and the other of the two donor units (e.g., a second donor unit) comprises greater than the number of platelets required to prepare two therapeutic dosage unit of platelets, but less than the number of platelets required to prepare three therapeutic dosage units of platelets. In some embodiments, two apheresis-derived platelet donor units are combined, and one of the two donor units comprises greater than the number of platelets required to prepare one therapeutic dosage unit of platelets, but less than the number of platelets required to prepare two therapeutic dosage units of platelets, and the other of the two donor units comprises greater than the number of platelets required to prepare three therapeutic dosage unit of platelets, but less than the number of platelets required to prepare four therapeutic dosage units of platelets. In some embodiments, two apheresis-derived platelet donor units are combined, and one of the two donor units comprises greater than the number of platelets required to prepare two therapeutic dosage units of platelets, but less than the number of platelets required to prepare three therapeutic dosage units of platelets, and the other of the two donor units comprises greater than the number of platelets required to prepare three therapeutic dosage units of platelets, but less than the number of platelets required to prepare four therapeutic dosage units of platelets. In some embodiments, two apheresis-derived platelet donor units are combined, and one of the two donor units comprises less than the number of platelets required to prepare one therapeutic dosage unit of platelets, and the other of the two donor units comprises greater than the number of platelets required to prepare one therapeutic dosage unit of platelets, but less than the number of platelets required to prepare two therapeutic dosage units of platelets. In some embodiments, two apheresis-derived platelet donor units are combined, and one of the two donor units comprises less than the number of platelets required to prepare one therapeutic dosage unit of platelets, and the other of the two donor units comprises greater than the number of platelets required to prepare two therapeutic dosage units of platelets, but less than the number of platelets required to prepare three therapeutic dosage units of platelets. In some embodiments, two apheresis-derived platelet donor units are combined, and one of the two donor units comprises less than the number of platelets required to prepare one therapeutic dosage unit of platelets, and the other of the two donor units comprises greater than the number of platelets required to prepare three therapeutic dosage units of platelets, but less than the number of platelets required to prepare four therapeutic dosage units of platelets. In some embodiments, at least one of the two donor units comprises at least about 125%, at least about 130%, at least about 135%, at least about 140%, at least about 145%, or at least about at least about 150% of the number of platelets required to prepare one platelet unit. In some embodiments, at least one of the two donor units comprises at least about 225%, at least about 230%, at least about 235%, at least about 240%, at least about 245%, or at least about at least about 250% of the number of platelets required to prepare one platelet unit. In some embodiments, at least one of the two donor units comprises at least about 325%, at least about 330%, at least about 335%, at least about 340%, at least about 345%, or at least about at least about 350% of the number of platelets required to prepare one platelet unit. In some embodiments, at least one donor units comprises less than about 98%, less than about 198%, less than about 298% or less than about 398% of the number of platelets required to prepare one platelet unit.

In some embodiments, at least one of two or more apheresis-derived platelet donor units is selected from an inventory log of an inventory of apheresis-collected platelet donor units, the inventory log comprising for each platelet donor unit a unique identifier, a donor blood type, a donation time and a quantity of platelets in the donor unit. In some embodiments, each of the two or more apheresis-derived platelet donor units is selected from the inventory log. In some embodiments, the two or more apheresis-derived platelet donor units are combined within about 7 days, within about 6 days, within about 5 days, within about 4 days, within about 3 days, within about 2 days, or within about 1 day from the time of collection. In some embodiments, the two or more apheresis-derived platelet donor units are combined within 20 hours from the time of collection (e.g., completed collection of each donor unit). In some embodiments, the two or more apheresis-derived platelet donor units are combined within 12 hours from the time of collection (e.g., completed collection of each donor unit). In some embodiments, the two or more apheresis-derived platelet donor units are combined within 6 hours from the time of collection (e.g., completed collection of each donor unit). In some embodiments, the two or more apheresis-derived platelet donor units are from donors of the same ABO blood type. In some embodiments, subjecting the pooled platelet composition to a pathogen inactivation process comprises treating the pooled pathogen composition with a pathogen inactivating compound. In some embodiments, the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540. In some embodiments, the pathogen inactivating compound is a psoralen. In some embodiments, the pathogen inactivating compound is amotosalen.

In some embodiments, the two or more platelet units (e.g., each of the two or more platelet units) comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ platelets. In some embodiments, each therapeutic dosage unit of platelets comprises at least 2.4×10¹¹ platelets, at least 2.5×10¹¹ platelets, at least 2.6×10¹¹ platelets, at least 2.7×10¹¹ platelets, at least 2.8×10¹¹ platelets, at least 2.9×10¹¹ platelets or at least about 3.0×10¹¹ or more platelets. In some embodiments, the platelet units each comprise at least about 3.0×10¹¹ platelets, and the pooled platelet preparation comprises at least about 6.2×10¹¹ platelets. In some embodiments, the platelet units each comprise at least about 3.0×10¹¹ platelets, and the pooled platelet preparation comprises at least about 9.3×10¹¹ platelets. In some embodiments, the platelet units each comprise at least about 3.0×10¹¹ platelets, and the pooled platelet preparation comprises at least about 12.4×10¹¹ platelets. In some embodiments, the platelet units each comprise at least about 3.0×10¹¹ platelets, and the pooled platelet preparation comprises at least about 15.5×10¹¹ platelets.

In some of the aforementioned embodiments, the therapeutic dosage units of platelets comprise a minimum number of platelets as defined by criteria (e.g., acceptance criteria) of a governmental agency, regulatory agency, institution and/or accrediting organization (e.g., governmental agency, regulatory agency, institution and/or accrediting organization for donated blood products (e.g., donated platelets)). In some embodiments, the therapeutic dosage units of platelets are prepared in the country of the governmental agency, regulatory agency, institution and/or accrediting organization defining the criteria of a therapeutic dosage unit of platelets. In some embodiments, at least about 75%, at least about 80%, at least about 85%, at least about 90%, at least about 95%, at least about 98%, at least about 99% or more of the therapeutic dosage units of platelets comprise the minimum number of platelets of a therapeutic dosage unit. In some embodiments, each of the therapeutic dosage units of platelets comprises the minimum number of platelets of a therapeutic dosage unit. In some embodiments, the minimum number of platelets in a therapeutic dosage unit is at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ platelets.

In another aspect, the present disclosure provides a therapeutic dosage unit of platelets prepared by the method of any of the aforementioned methods.

In another aspect, the present disclosure provides, a method for collecting a platelet donation by apheresis from a donor, comprising: a) maintaining an inventory log of an inventory of apheresis-collected platelet preparations, the inventory log comprising for each platelet preparation a unique identifier, a donor blood type, a donation date and a quantity of platelets in the preparation, b) identifying a donor suitable for donating platelets by apheresis, c) selecting from the inventory log one or more platelet preparations suitable for combining with a new platelet donation to be collected by apheresis from the donor, d) determining a target volume of the new platelet donation, wherein the target volume optimizes the number of platelet units that can be prepared from a combination comprising the new platelet donation and the one or more selected platelet preparations; and e) collecting the target volume of the new platelet donation by apheresis from the donor. In some embodiments, determining a target volume of the new platelet donation comprises determining a target yield of platelets for the new platelet donation. In some embodiments, the method further comprises determining a duration time for the collection of the new platelet donation by apheresis. In some embodiments, the method further comprises: f) combining the one or more selected platelet preparations and the new platelet donation to yield a pooled platelet preparation; and g) separating the pooled platelet preparation into a plurality of platelet units (e.g., therapeutic dosage units) each in an individual container. In some embodiments, each of the platelet units in the plurality of platelet units comprises a therapeutic dose (e.g., therapeutic dose of platelets) suitable for infusion into a human subject in need thereof. In some embodiments, the plurality of platelet units comprises a greater number of platelet units than the total number of platelet units that can be prepared from the one or more selected platelet preparations and the new platelet donation without combining the one or more selected platelet preparations and the new platelet donation.

In some embodiments, each of the one or more selected platelet preparations comprises less than the number of platelets required to prepare one therapeutic dose. In some embodiments, each of the one or more selected platelet preparations comprises greater than the number of platelets required to prepare one therapeutic dose, but less than the number of platelets required to prepare two therapeutic doses. In some embodiments, each of the one or more selected platelet preparations comprises greater than the number of platelets required to prepare two therapeutic doses, but less than the number of platelets required to prepare three therapeutic doses. In some embodiments, each of the one or more selected platelet preparations comprises greater than the number of platelets required to prepare three therapeutic doses, but less than the number of platelets required to prepare four therapeutic doses.

In some embodiments, the new platelet donation comprises less than the number of platelets required to prepare one therapeutic dose. In some embodiments, the new platelet donation comprises greater than the number of platelets required to prepare one therapeutic dose, but less than the number of platelets required to prepare two therapeutic doses. In some embodiments, the new platelet donation comprises greater than the number of platelets required to prepare two therapeutic doses, but less than the number of platelets required to prepare three therapeutic doses. In some embodiments, the new platelet donation comprises greater than the number of platelets required to prepare three therapeutic doses, but less than the number of platelets required to prepare four therapeutic doses.

In some embodiments, the one or more selected platelet preparations comprises less than the number of platelets required to prepare one platelet unit. In some embodiments, the target volume of the new platelet donation is a volume comprising a sufficient number of platelets to prepare two, to prepare three, or to prepare four platelet units after combining the one or more selected platelet preparations and the new platelet donation and separating the resulting pooled platelet preparation into a plurality of platelet units. In some embodiments, the new platelet donation comprises a sufficient number of platelets that, when combined with the one or more selected platelet preparation, yields a pooled platelet preparation comprising a sufficient number of platelets to separate into two, into three, or into four platelet units. In some embodiments, collecting the new platelet donation from the donor comprises collecting a sufficient number of platelets to prepare two, to prepare three, or to prepare four platelet units from the pooled preparation.

In some embodiments, the one or more selected platelet preparations comprises greater than the number of platelets required to prepare one platelet unit, but less than the number of platelets required to prepare two platelet units. In some embodiments, the one or more selected platelet preparations comprises at least about 125%, at least about 130%, at least about 135%, at least about 140%, at least about 145%, or at least about at least about 150% of the number of platelets required to prepare one platelet unit, but less than the number of platelets required to prepare two platelet units. In some embodiments, the target volume of the new platelet donation is a volume comprising a sufficient number of platelets to prepare two, to prepare three, to prepare four, or to prepare five platelet units after combining the one or more selected platelet preparations and the new platelet donation and separating the resulting pooled platelet preparation into a plurality of platelet units. In some embodiments, the new platelet donation comprises a sufficient number of platelets that, when combined with the one or more selected platelet preparation, yields a pooled platelet preparation comprising a sufficient number of platelets to separate into two, into three, into four, or into five platelet units. In some embodiments, collecting the new platelet donation from the donor comprises collecting a sufficient number of platelets to prepare two, to prepare three, to prepare four, or to prepare five platelet units from the pooled preparation.

In some embodiments, the one or more selected platelet preparations comprises greater than the number of platelets required to prepare two platelet units, but less than the number of platelets required to prepare three platelet units. In some embodiments, the one or more selected platelet preparations comprises at least about 225%, at least about 230%, at least about 235%, at least about 240%, at least about 245%, or at least about at least about 250% of the number of platelets required to prepare one platelet unit, but less than the number of platelets required to prepare three platelet units. In some embodiments, the target volume of the new platelet donation is a volume comprising a sufficient number of platelets to prepare three, to prepare four, to prepare five, or to prepare six platelet units after combining the one or more selected platelet preparations and the new platelet donation and separating the resulting pooled platelet preparation into a plurality of platelet units. In some embodiments, the new platelet donation comprises a sufficient number of platelets that, when combined with the one or more selected platelet preparation, yields a pooled platelet preparation comprising a sufficient number of platelets to separate into three, into four, into five, or into six platelet units. In some embodiments, collecting the new platelet donation from the donor comprises collecting a sufficient number of platelets to prepare three, to prepare four, to prepare five, or to prepare six platelet units from the pooled preparation.

In some embodiments, the one or more selected platelet preparations comprises greater than the number of platelets required to prepare three platelet units, but less than the number of platelets required to prepare four platelet units. In some embodiments, the one or more selected platelet preparations comprises at least about 325%, at least about 330%, at least about 335%, at least about 340%, at least about 345%, or at least about at least about 350% of the number of platelets required to prepare one platelet unit, but less than the number of platelets required to prepare four platelet units. In some embodiments, the target volume of the new platelet donation is a volume comprising a sufficient number of platelets to prepare to prepare four, to prepare five, to prepare six, or to prepare seven platelet units after combining the one or more selected platelet preparations and the new platelet donation and separating the resulting pooled platelet preparation into a plurality of platelet units. In some embodiments, the new platelet donation comprises a sufficient number of platelets that, when combined with the one or more selected platelet preparation, yields a pooled platelet preparation comprising a sufficient number of platelets to separate into four, into five, into six, or into seven platelet units. In some embodiments, collecting the new platelet donation from the donor comprises collecting a sufficient number of platelets to prepare four, to prepare five, to prepare six, or to prepare seven platelet units from the pooled preparation.

In some embodiments, each of the one or more selected platelet preparations and the new platelet donation comprises greater than the number of platelets required to prepare one therapeutic dose, but less than the number of platelets required to prepare two therapeutic doses. In some embodiments, each of the one or more selected platelet preparations and the new platelet donation comprises greater than the number of platelets required to prepare two therapeutic dosage units of platelets, but less than the number of platelets required to prepare three therapeutic dosage units of platelets. In some embodiments, the one or more selected platelet preparations comprise greater than the number of platelets required to prepare one therapeutic dose, but less than the number of platelets required to prepare two therapeutic doses and the new platelet donation comprises less than the number of platelets required to prepare one therapeutic doses. In some embodiments, the one or more selected platelet preparations comprise greater than the number of platelets required to prepare one therapeutic dose, but less than the number of platelets required to prepare two therapeutic doses and the new platelet donation comprises greater than the number of platelets required to prepare two therapeutic doses, but less than the number of platelets required to prepare three therapeutic doses. In some embodiments, the one or more selected platelet preparations comprise greater than the number of platelets required to prepare one therapeutic dose, but less than the number of platelets required to prepare two therapeutic doses and the new platelet donation comprises greater than the number of platelets required to prepare three therapeutic doses, but less than the number of platelets required to prepare four therapeutic doses. In some embodiments, the one or more selected platelet preparations comprise greater than the number of platelets required to prepare two therapeutic doses, but less than the number of platelets required to prepare three therapeutic doses and the new platelet donation comprises less than the number of platelets required to prepare one therapeutic dose. In some embodiments, the one or more selected platelet preparations comprise greater than the number of platelets required to prepare two therapeutic doses, but less than the number of platelets required to prepare three therapeutic doses and the new platelet donation comprises greater than the number of platelets required to prepare one therapeutic dose, but less than the number of platelets required to prepare two therapeutic doses. In some embodiments, the one or more selected platelet preparations comprise greater than the number of platelets required to prepare two therapeutic doses, but less than the number of platelets required to prepare three therapeutic doses and the new platelet donation comprises greater than the number of platelets required to prepare three therapeutic doses, but less than the number of platelets required to prepare four therapeutic doses.

In some embodiments, the one or more selected platelet preparations and the new platelet donation are combined within about 7 days, within about 6 days, within about 5 days, within about 4 days, within about 3 days, within about 2 days, or within about 1 day from the time of collection. In some embodiments, the one or more selected platelet preparations and the new platelet donation are combined within 20 hours from the time of collection. In some embodiments, the one or more selected platelet preparations and the new platelet donation are combined within 12 hours from the time of collection. In some embodiments, the one or more selected platelet preparations and the new platelet donation are combined within 6 hours from the time of collection. In some embodiments, the one or more selected platelet preparations and the new platelet donation are from donors of the same ABO blood type.

In some embodiments, the platelet units (e.g., each platelet unit) in the plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ platelets. In some embodiments, each platelet unit comprises at least 2.4×10¹¹ platelets. In some embodiments, each platelet unit comprises at least 2.6×10¹¹ platelets. In some embodiments, each platelet unit comprises at least 3.0×10¹¹ platelets. In some embodiments, the platelet units each comprise at least about 3.0×10¹¹ platelets, and the pooled platelet preparation comprises at least about 6.2×10¹¹ platelets. In some embodiments, the platelet units each comprise at least about 3.0×10¹¹ platelets, and the pooled platelet preparation comprises at least about 9.3×10¹¹ platelets. In some embodiments, the platelet units each comprise at least about 3.0×10¹¹ platelets, and the pooled platelet preparation comprises at least about 12.4×10¹¹ platelets. In some embodiments, the platelet units each comprise at least about 3.0×10¹¹ platelets, and the pooled platelet preparation comprises at least about 15.5×10¹¹ platelets.

In some embodiments, the method further comprises subjecting the pooled platelet preparation to a pathogen inactivation process. In some embodiments, subjecting the pooled platelet preparation to a pathogen inactivation process comprises treating the pooled platelet preparation with a pathogen inactivating compound. In some embodiments, the method further comprises subjecting each of the one or more selected platelet preparations and the new platelet donation to a pathogen inactivation process. In some embodiments, subjecting the one or more selected platelet preparations and the new platelet donation to a pathogen inactivation process comprises treating the one or more selected platelet preparations and the new platelet donation with a pathogen inactivating compound, In some embodiments, the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540. In some embodiments, the pathogen inactivating compound is a psoralen. In some embodiments, the pathogen inactivating compound is amotosalen.

In some embodiments, the platelet units comprise a minimum number of platelets as defined by criteria (e.g., acceptance criteria) of a governmental agency, regulatory agency, institution and/or accrediting organization (e.g., governmental agency, regulatory agency, institution and/or accrediting organization for donated blood products (e.g., donated platelets)). In some embodiments, the platelet units of platelets are prepared in the country of the governmental agency, regulatory agency, institution and/or accrediting organization defining the criteria of a platelet unit. In some embodiments, at least about 75%, at least about 80%, at least about 85%, at least about 90%, at least about 95%, at least about 98%, at least about 99% or more of the platelets units comprise the minimum number of platelets of a platelet unit. In some embodiments, each of the platelet units comprises the minimum number of platelets of a therapeutic dosage unit. In some embodiments, the minimum number of platelets in a therapeutic dosage unit is at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ platelets.

In another aspect, the present disclosure provides a platelet unit suitable for infusion into a human subject, wherein the platelet unit is selected from a plurality of platelet units prepared by any of the aforementioned methods.

In another aspect, the present disclosure provides a therapeutic dosage unit of platelets suitable for infusion into a subject, wherein the therapeutic dosage unit comprises pooled apheresis-derived platelets from two or more donors, and wherein the pooled apheresis-derived platelets have been treated with a pathogen inactivating compound. In some embodiments, the apheresis-derived platelets have been treated with the pathogen inactivating compound prior to pooling. In some embodiments, the apheresis-derived platelets have been treated with the pathogen inactivating compound after pooling. In some embodiments, the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540. In some embodiments, the pathogen inactivating compound is a psoralen. In some embodiments, the pathogen inactivating compound is amotosalen. In some embodiments, the apheresis-derived platelets are from donors of the same ABO blood type. In some embodiments, the apheresis-derived platelets are from donors of the same ABO and Rh type. In some embodiments, the therapeutic dosage unit of platelets comprises at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets, at least about 3.0×10¹¹ or more platelets. In some embodiments, the therapeutic dosage unit of platelets comprises at least 2.4×10¹¹ platelets. In some embodiments, the therapeutic dosage unit of platelets comprises at least 2.6×10¹¹ platelets. In some embodiments, the therapeutic dosage unit of platelets comprises at least 3.0×10¹¹ platelets.

In some embodiments, the therapeutic dosage unit of platelets comprises a minimum number (e.g., at least a minimum number) of platelets as defined by criteria (e.g., acceptance criteria) of a governmental agency, regulatory agency, institution and/or accrediting organization (e.g., governmental agency, regulatory agency, institution and/or accrediting organization for donated blood products (e.g., donated platelets)). In some embodiments, the therapeutic dosage unit of platelets is prepared in the country of the governmental agency, regulatory agency, institution and/or accrediting organization defining the criteria of a therapeutic dosage unit of platelets.

In another aspect, the present disclosure provides a method of infusing platelets into a subject in need thereof, comprising infusing into the subject an aforementioned platelet unit or an aforementioned therapeutic dosage unit of platelets.

In another aspect, the present disclosure provides a computer-implemented method for optimization of platelet units (e.g., platelet units for infusion), comprising: at a computer system including one or more processors and memory, receiving one or more values associated with one or more properties of a platelet donation; instructing to search, based on the received one or more values, a plurality of values associated with representations of a plurality of platelet preparations; determining, based on the search results, a volume of the platelet donation; and providing the determined volume of the platelet donation, wherein the determined volume of the platelet donation enables optimization of platelet units (e.g., platelet units for infusion). In some embodiments, determining a volume of the platelet donation further comprises determining a platelet yield (e.g., target platelet yield) of the platelet donation. In some embodiments, determining a volume of the platelet donation further comprises determining a duration of the platelet donation. In some embodiments, optimization of platelet units comprises optimizing the number of platelet units that can be prepared (e.g., from the platelet donation and the plurality of platelet preparations, relative to the process for preparing platelet units before optimizing). In some embodiments, optimization of platelet units comprises reducing excess platelet platelets per platelet unit (e.g., relative to the process for preparing platelet units before optimizing). In some embodiments, optimization of platelet units comprises reducing or eliminating platelet waste (e.g., relative to the process for preparing platelet units before optimizing). In some embodiments, optimization of platelet units comprises improving or increasing the number of platelet units prepared (e.g., from the platelet donation and the plurality of platelet preparations, relative to the process for preparing platelet units before optimizing). In some embodiments, optimization of platelet units comprises increasing the efficiency of preparing platelet units (e.g., relative to the process for preparing platelet units before optimizing). In some embodiments, optimization of platelet units comprises obtaining a target value (e.g., number) of platelets per platelet unit.

In some embodiments, at least one of the platelet donation and the plurality of platelet preparations comprises apheresis-derived platelets. In some embodiments, the platelet donation comprises platelets being currently collected from a donor or platelets to be collected from a donor. In some embodiments, the one or more properties of the platelet donation comprises: a blood type, a donation time (e.g., date, time, date and time), number of platelets per unit volume, and optionally a maximum platelet donation volume. In some embodiments, the value of the blood type includes one of: A, B, AB, or O.

In some embodiments, instructing to search the plurality of values associated with representations of a plurality of platelet preparations comprises: obtaining one or more criteria for searching using the one or more received values associated with one or more properties of the platelet donation; instructing to search the plurality of values associated with the representations of the plurality of platelet preparations based on the one or more criteria for searching; and obtaining one or more candidate representations of platelet preparations from the representations of the plurality of platelet preparations, the one or more candidate representations of platelet preparations having values that satisfy the one or more criteria for searching. In some embodiments, the criteria for searching include one or more threshold conditions of one or more of: a blood type, a donation time, a platelet volume and number of platelets per unit volume. In some embodiments, obtaining the one or more candidate representations of platelet preparations comprises filtering the representations of the plurality of platelet preparations based on the one or more criteria for searching. In some embodiments, the representations of the plurality of platelet preparations include at least one of: platelet preparations stored in a platelet preparation inventory, platelet preparations being currently collected from a plurality of donors, and platelet preparations to be collected from a plurality of donors.

In some embodiments, determining the volume of the platelet donation comprises: obtaining one or more values corresponding to one or more candidate representations of platelet preparations provided by the search results; obtaining one or more criteria for platelet units, the criteria for platelet units enabling optimization of platelet units; determining, based on the one or more values corresponding to one or more candidate representations of platelet preparations and the one or more criteria for platelet units, one or more platelet preparations to select from the one or more candidate representations of the plurality of platelet preparations; and obtaining the volume of the platelet donation based on the determined platelet preparation. In some embodiments, providing the determined volume of the platelet donation comprises at least one of: displaying the determined volume of the platelet donation on a display of the computer system; sending a notification providing the determined volume of the platelet donation; and providing the determined volume of the platelet donation to an apheresis system. In some embodiments, determining the volume of the platelet donation further comprises determining a platelet yield (e.g., target platelet yield) of the platelet donation. In some embodiments, determining the volume of the platelet donation further comprises determining a duration time for the platelet donation. In some embodiments, providing the determined volume of the platelet donation further comprises at least one of: displaying the determined platelet yield and/or duration time of the platelet donation on a display of the computer system; sending a notification providing the determined platelet yield and/or duration time of the platelet donation; and providing the determined platelet yield and/or duration time of the platelet donation to an apheresis system.

In some embodiments, the method further comprises combining the platelet donation and one or more platelet preparations selected from the representations of the plurality of platelet preparations to yield a pooled platelet preparation. In some embodiments, the method further comprises separating the pooled platelet preparation into a plurality of platelet units (e.g., therapeutic dosage units of platelets) each in an individual container. In some embodiments, the platelet units (e.g., each platelet unit) in the plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ platelets. In some embodiments, each platelet unit comprises at least 2.4×10¹¹ platelets. In some embodiments, each platelet unit comprises at least 2.6×10¹¹ platelets. In some embodiments, each platelet unit comprises at least 3.0×10¹¹ platelets. In some embodiments, the one or more selected platelet preparations and the platelet donation are combined within 20 hours from the time of collection. In some embodiments, the one or more selected platelet preparations and the platelet donation are combined within 12 hours from the time of collection. In some embodiments, the one or more selected platelet preparations and the platelet donation are combined within 6 hours from the time of collection. In some embodiments, the one or more selected platelet preparations and the platelet donation are from donors of the same ABO blood type. In some embodiments, the method further comprises subjecting the pooled platelet preparation to a pathogen inactivation process. In some embodiments, subjecting the pooled platelet preparation to a pathogen inactivation process comprises treating the pooled platelet preparation with a pathogen inactivating compound. In some embodiments, the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540. In some embodiments, the pathogen inactivating compound is a psoralen. In some embodiments, the pathogen inactivating compound is amotosalen.

In some embodiments, the plurality of platelet units comprises a greater number of platelet units than the total number of platelet units that can be prepared from the one or more selected platelet preparations and the platelet donation without combining the one or more selected platelet preparations and the platelet donation.

In some embodiments, each of the one or more selected platelet preparations comprises less than the number of platelets required to prepare one platelet unit. In some embodiments, each of the one or more selected platelet preparations comprises greater than the number of platelets required to prepare one platelet unit, but less than the number of platelets required to prepare two platelet units. In some embodiments, each of the one or more selected platelet preparations comprises greater than the number of platelets required to prepare two platelet units, but less than the number of platelets required to prepare three platelet units. In some embodiments, each of the one or more selected platelet preparations comprises greater than the number of platelets required to prepare three platelet units, but less than the number of platelets required to prepare four therapeutic platelet units.

In some embodiments, the platelet donation comprises less than the number of platelets required to prepare one platelet unit. In some embodiments, the platelet donation comprises greater than the number of platelets required to prepare one platelet unit, but less than the number of platelets required to prepare platelet units. In some embodiments, the platelet donation comprises greater than the number of platelets required to prepare two platelet units, but less than the number of platelets required to prepare three platelet units. In some embodiments, the platelet donation comprises greater than the number of platelets required to prepare three platelet units, but less than the number of platelets required to prepare four platelet units.

In some embodiments, each of the one or more selected platelet preparations and the platelet donation comprises greater than the number of platelets required to prepare one platelet unit, but less than the number of platelets required to prepare two platelet units. In some embodiments, each of the one or more selected platelet preparations and the platelet donation comprises greater than the number of platelets required to prepare platelet units, but less than the number of platelets required to prepare platelet units. In some embodiments, the one or more selected platelet preparations comprise greater than the number of platelets required to prepare one platelet unit, but less than the number of platelets required to prepare two platelet units and the platelet donation comprises less than the number of platelets required to prepare one platelet unit. In some embodiments, the one or more selected platelet preparations comprise greater than the number of platelets required to prepare one platelet unit, but less than the number of platelets required to prepare two platelet units and the platelet donation comprises greater than the number of platelets required to prepare two platelet unit, but less than the number of platelets required to prepare three platelet unit. In some embodiments, the one or more selected platelet preparations comprise greater than the number of platelets required to prepare one platelet unit, but less than the number of platelets required to prepare two platelet units and the platelet donation comprises greater than the number of platelets required to prepare three platelet unit, but less than the number of platelets required to prepare four platelet unit. In some embodiments, the one or more selected platelet preparations comprise greater than the number of platelets required to prepare two platelet units, but less than the number of platelets required to prepare three platelet units and the platelet donation comprises less than the number of platelets required to prepare one platelet unit. In some embodiments, the one or more selected platelet preparations comprise greater than the number of platelets required to prepare two platelet units, but less than the number of platelets required to prepare three platelet units and the platelet donation comprises greater than the number of platelets required to prepare one platelet unit, but less than the number of platelets required to prepare two platelet units. In some embodiments, the one or more selected platelet preparations comprise greater than the number of platelets required to prepare two platelet units, but less than the number of platelets required to prepare three platelet units and the platelet donation comprises greater than the number of platelets required to prepare three platelet units, but less than the number of platelets required to prepare four platelet units.

In another aspect, the present disclosure provides a non-transitory computer readable medium having instructions stored thereon for optimization of platelet units (e.g., platelet units for infusion), the instructions, when executed by one or more processors of a computer system, cause the computer system to: receive one or more values associated with one or more properties of a platelet donation; instruct to search, based on the received one or more values, a plurality of values associated with representations of a plurality of platelet preparations; determine, based on the search results, a volume of the platelet donation; and provide the determined volume of the platelet donation, wherein the determined volume of the platelet donation enables optimization of platelet units (e.g., platelet units for infusion). In some embodiments, determining a volume of the platelet donation further comprises determining a platelet yield (e.g., target platelet yield) of the platelet donation, and providing the determined platelet yield of the platelet donation, wherein the determined platelet yield of the platelet donation enables optimization of platelet units. In some embodiments, determining a volume of the platelet donation further comprises determining a duration of the platelet donation, and providing the determined duration of the platelet donation, wherein the determined duration of the platelet donation enables optimization of platelet units.

In another aspect, the present disclosure provides a system for optimization of platelet units (e.g., platelet units for infusion), comprising: one or more processors; and memory having instructions stored thereon, the instruction, when executed by the one or more processors, cause the computer system to: receive one or more values associated with one or more properties of a platelet donation; instruct to search, based on the received one or more values, a plurality of values associated with representations of a plurality of platelet preparations; determine, based on the search results, a volume of the platelet donation; and provide the determined volume of the platelet donation, wherein the determined volume of the platelet donation enables optimization of platelet units (e.g., platelet units for infusion). In some embodiments, determining a volume of the platelet donation further comprises determining a platelet yield (e.g., target platelet yield) of the platelet donation, and providing the determined platelet yield of the platelet donation, wherein the determined platelet yield of the platelet donation enables optimization of platelet units. In some embodiments, determining a volume of the platelet donation further comprises determining a duration of the platelet donation, and providing the determined duration of the platelet donation, wherein the determined duration of the platelet donation enables optimization of platelet units. In some embodiments, the computer system is integrated with an apheresis system.

In another aspect, the present disclosure provides a computer-implemented method for optimization of platelet units (e.g., platelet units for infusion), comprising: at a computer system including one or more processors and memory, receiving one or more values associated with one or more properties of a first platelet preparation; instructing to search, based on the received one or more values, a plurality of values associated with representations of a plurality of platelet preparations; selecting, based on the search results, a second platelet preparation from the representations of a plurality of platelet preparations; and providing the selected second platelet preparation, wherein the selected second platelet preparation enables optimization of platelet units (e.g., platelet units for infusion). In some embodiments, optimization of platelet units comprises optimizing the number of platelet units that can be prepared (e.g., from the platelet donation and the plurality of platelet preparations, relative to the process for preparing platelet units before optimizing). In some embodiments, optimization of platelet units comprises reducing excess platelet platelets per platelet unit (e.g., relative to the process for preparing platelet units before optimizing). In some embodiments, optimization of platelet units comprises reducing or eliminating platelet waste (e.g., relative to the process for preparing platelet units before optimizing). In some embodiments, optimization of platelet units comprises improving or increasing the number of platelet units prepared (e.g., from the platelet donation and the plurality of platelet preparations, relative to the process for preparing platelet units before optimizing). In some embodiments, optimization of platelet units comprises increasing the efficiency of preparing platelet units (e.g., relative to the process for preparing platelet units before optimizing). In some embodiments, optimization of platelet units comprises obtaining a target value (e.g., number) of platelets per platelet unit.

In some embodiments, at least one of the first platelet preparation and the plurality of platelet preparations comprises apheresis-derived platelets. In some embodiments, the first platelet preparation comprises a platelet preparation stored in a platelet preparation inventory. In some embodiments, the one or more properties of the first platelet preparation comprises: a blood type, a donation time (e.g., date, time, date and time), a volume, and number of platelets per unit volume. In some embodiments, the value of the blood type includes one of: A, B, AB, or O.

In some embodiments, instructing to search the plurality of values associated with representations of a plurality of platelet preparations comprises: obtaining one or more criteria for searching using the one or more received values associated with one or more properties of the first platelet preparation; instructing to search the plurality of values associated with the representations of the plurality of platelet preparations based on the one or more criteria for searching; and obtaining one or more candidate representations of platelet preparations from the representations of the plurality of platelet preparations, the one or more candidate representations of platelet preparations having values that satisfy the one or more criteria for searching. In some embodiments, the criteria for searching include one or more threshold conditions of one or more of: a blood type, a donation time, a volume, and number of platelets per unit volume. In some embodiments, obtaining the one or more candidate representations of platelet preparations comprises filtering the representations of the plurality of platelet preparations based on the one or more criteria for searching. In some embodiments, the representations of the plurality of platelet preparations include at least one of: platelet preparations stored in a platelet preparation inventory, platelet preparations being currently collected from a plurality of donors, and platelet preparations to be collected from a plurality of donors.

In some embodiments, selecting the second platelet preparation comprises: obtaining one or more values corresponding to one or more candidate representations of platelet preparations provided by the search results; obtaining one or more criteria for platelet units, the criteria for platelet units enabling optimization of platelet units; and selecting, based on the one or more values corresponding to one or more candidate representations of platelet preparations and the one or more criteria for platelet units, a second platelet preparation from the one or more candidate representations of platelet preparations. In some embodiments, providing the selected second platelet preparation comprises at least one of: displaying the selected second platelet preparation on a display of the computer system; and sending a notification providing the selected second platelet preparation; and providing an identification of the selected second platelet preparation to an apheresis system.

In some embodiments, the method further comprises combining the first platelet preparation and selected second platelet preparation to yield a pooled platelet preparation. In some embodiments, the method further comprises separating the pooled platelet preparation into a plurality of platelet units (e.g., therapeutic dosage units of platelets) each in an individual container. In some embodiments, the platelet units (e.g., each platelet unit) in the plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ platelets. In some embodiments, each platelet unit comprises at least 2.4×10¹¹ platelets. In some embodiments, each platelet unit comprises at least 2.6×10¹¹ platelets. In some embodiments, each platelet unit comprises at least 3.0×10¹¹ platelets. In some embodiments, the first platelet preparation and selected second platelet preparation are combined within 20 hours from the time of collection. In some embodiments, the first platelet preparation and selected second platelet preparation are combined within 12 hours from the time of collection. In some embodiments, the first platelet preparation and selected second platelet preparation are combined within 6 hours from the time of collection. In some embodiments, the first platelet preparation and selected second platelet preparation are from donors of the same ABO blood type. In some embodiments, the method further comprises subjecting the pooled platelet preparation to a pathogen inactivation process. In some embodiments, subjecting the pooled platelet preparation to a pathogen inactivation process comprises treating the pooled platelet preparation with a pathogen inactivating compound. In some embodiments, the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540. In some embodiments, the pathogen inactivating compound is a psoralen. In some embodiments, the pathogen inactivating compound is amotosalen.

In some embodiments, the plurality of platelet units comprises a greater number of platelet units than the total number of platelet units that can be prepared from the first platelet preparation and selected second platelet preparation without combining the first platelet preparation and selected second platelet preparation.

In some embodiments, the selected second platelet preparation comprises less than the number of platelets required to prepare one platelet unit. In some embodiments, the selected second platelet preparation comprises greater than the number of platelets required to prepare one platelet unit, but less than the number of platelets required to prepare two platelet units. In some embodiments, the selected second platelet preparation comprises greater than the number of platelets required to prepare two platelet units, but less than the number of platelets required to prepare three platelet units. In some embodiments, the selected second platelet preparation comprises greater than the number of platelets required to prepare three platelet units, but less than the number of platelets required to prepare four therapeutic platelet units.

In some embodiments, the first platelet preparation comprises less than the number of platelets required to prepare one platelet unit. In some embodiments, the first platelet preparation comprises greater than the number of platelets required to prepare one platelet unit, but less than the number of platelets required to prepare platelet units. In some embodiments, the first platelet preparation comprises greater than the number of platelets required to prepare two platelet units, but less than the number of platelets required to prepare three platelet units. In some embodiments, the first platelet preparation comprises greater than the number of platelets required to prepare three platelet units, but less than the number of platelets required to prepare four platelet units.

In some embodiments, each of the selected second platelet preparation and the first platelet preparation comprises greater than the number of platelets required to prepare one platelet unit, but less than the number of platelets required to prepare two platelet units. In some embodiments, each of the selected second platelet preparation and the first platelet preparation comprises greater than the number of platelets required to prepare platelet units, but less than the number of platelets required to prepare platelet units. In some embodiments, the selected second platelet preparation comprises greater than the number of platelets required to prepare one platelet unit, but less than the number of platelets required to prepare two platelet units and the first platelet preparation comprises less than the number of platelets required to prepare one platelet unit. In some embodiments, the selected second platelet preparation comprises greater than the number of platelets required to prepare one platelet unit, but less than the number of platelets required to prepare two platelet units and the first platelet preparation comprises greater than the number of platelets required to prepare two platelet unit, but less than the number of platelets required to prepare three platelet units. In some embodiments, the selected second platelet preparation comprises greater than the number of platelets required to prepare one platelet unit, but less than the number of platelets required to prepare two platelet units and the first platelet preparation comprises greater than the number of platelets required to prepare three platelet units, but less than the number of platelets required to prepare four platelet units. In some embodiments, the selected second platelet preparation comprises greater than the number of platelets required to prepare two platelet units, but less than the number of platelets required to prepare three platelet units and the first platelet preparation comprises less than the number of platelets required to prepare one platelet unit. In some embodiments, the selected second platelet preparation comprises greater than the number of platelets required to prepare two platelet units, but less than the number of platelets required to prepare three platelet units and the first platelet preparation comprises greater than the number of platelets required to prepare one platelet unit, but less than the number of platelets required to prepare two platelet units. In some embodiments, the selected second platelet preparation comprises greater than the number of platelets required to prepare two platelet units, but less than the number of platelets required to prepare three platelet units and the first platelet preparation comprises greater than the number of platelets required to prepare three platelet units, but less than the number of platelets required to prepare four platelet units.

In another aspect, the present disclosure provides a non-transitory computer readable medium having instructions stored thereon for optimization of platelet units (e.g., platelet units for infusion), the instructions, when executed by one or more processors of a computer system, cause the computer system to: receive one or more values associated with one or more properties of a first platelet preparation; instruct to search, based on the received one or more values, a plurality of values associated with representations of a plurality of platelet preparations; select, based on the search results, a second platelet preparation from the representations of a plurality of platelet preparations; and provide the selected second platelet preparation, wherein the selected second platelet preparation enables optimization of platelet units (e.g., platelet units for infusion).

In another aspect, the present disclosure provides, a system for optimization of platelet units (e.g., platelet units for infusion), comprising: one or more processors; and memory having instructions stored thereon, the instruction, when executed by the one or more processors, cause the computer system to: receive one or more values associated with one or more properties of a first platelet preparation; instruct to search, based on the received one or more values, a plurality of values associated with representations of a plurality of platelet preparations; select, based on the search results, a second platelet preparation from the representations of a plurality of platelet preparations; and provide the selected second platelet preparation, wherein the selected second platelet preparation enables optimization of platelet units (e.g., platelet units for infusion). In some embodiments, the computer system is integrated with an apheresis system.

In another aspect, the present disclosure provides a processing set for preparing a plurality of platelet units from two or more platelet donations, wherein each of the platelet units is pathogen-inactivated and suitable for infusion into a subject, the processing set comprising: a) a first container that contains a pathogen inactivation compound (PIC) and is suitable for combining the two or more platelet donations with the PIC; b) a second container, coupled to the first container, within which the two or more platelet donations in admixture with the PIC can be photochemically inactivated under sterile conditions, wherein the volume of the two or more platelet donations is sufficient to yield the plurality of platelet units; c) a compound absorption device (CAD) coupled to the second container such that the two or more photochemically inactivated platelet donations can be transferred from the second container to the compound absorption device under sterile conditions; and d) two or more third containers, each of which is coupled to the compound absorption device such that the two or more photochemically inactivated platelet donations can be transferred from the compound absorption device to the two or more third containers under sterile conditions to provide the plurality of platelet units, wherein the plurality of platelet units comprises a greater number of platelet units than the total number of platelet units that can be prepared from the two or more donations without combining the two or more platelet donations. In some embodiments, the PIC is a psoralen. In some embodiments, the PIC is amotosalen. In some embodiments, the platelet units in the plurality of platelet units each comprise a therapeutic dose of platelets.

In some embodiments, the processing set comprises three or more third containers, each of which is coupled to the compound absorption device such that the two or more photochemically inactivated platelet donations can be transferred from the compound absorption device to the three or more third containers under sterile conditions to provide the plurality of pathogen-inactivated platelet units. In some embodiments, the processing set comprises three third containers, each of which is coupled to the compound absorption device such that the two or more photochemically inactivated platelet donations can be transferred from the compound absorption device to the three third containers under sterile conditions to provide the plurality of pathogen-inactivated platelet units. In some embodiments, the first container is suitable for sterile coupling with an additional container. In some embodiments, the processing set further comprises an additional container suitable for containing one or more of the two or more platelet donations, wherein the additional container is coupled to the first container such that the one or more platelet donations of the additional container can be transferred from the additional container to the first container under sterile conditions. In some embodiments, the additional container is suitable for containing a platelet donation obtained by apheresis. In some embodiments, the additional container is suitable for sterile coupling with a second additional container. In some embodiments, the second additional container contains one or more of the two or more platelet donations, and wherein the additional container is suitable for sterile coupling with the second additional container such that the one or more platelet donations of the second additional container can be transferred from the second additional container to the additional container under sterile conditions.

In some embodiments, the processing set further comprises a second additional container suitable for containing one or more of the two or more platelet donations, wherein the second additional container is coupled to the additional container such that the one or more platelet donations of the second additional container can be transferred from the second additional container to the additional container under sterile conditions. In some embodiments, the additional container is suitable for containing a platelet donation obtained by apheresis from a first donor, and wherein the second additional container is suitable for containing a platelet donation obtained by apheresis from a second donor.

In another aspect, the present disclosure provides a kit comprising (a) a processing set, and (b) instructions for using the processing set to prepare a plurality of platelet units from two or more platelet donations, wherein each of the platelet units is pathogen-inactivated and suitable for infusion into a subject, wherein the plurality of platelet units comprises a greater number of platelet units than can be prepared from the two or more platelet donations without combining the two or more platelet donations, and wherein the processing set comprises: 1) a first container that contains a pathogen inactivation compound (PIC) and is suitable for combining the two or more platelet donations with the PIC; 2) a second container, coupled to the first container, within which the two or more platelet donations in admixture with the PIC can be photochemically inactivated under sterile conditions, wherein the volume of the two or more platelet donations is sufficient to yield the plurality of platelet units; 3) a compound absorption device (CAD) coupled to the second container such that the two or more photochemically inactivated platelet donations can be transferred from the second container to the compound absorption device under sterile conditions; and 4) two or more third containers, each of which is coupled to the compound absorption device such that the two or more photochemically inactivated platelet donations can be transferred from the compound absorption device to the two or more third containers under sterile conditions to provide the plurality of platelet units. In some embodiments, the kit comprises instructions for using the processing set to prepare a plurality of platelet units from two or more platelet concentrates, wherein each of the platelet units is pathogen-inactivated and suitable for infusion into a subject, wherein the plurality of platelet units comprises a greater number of platelet units than can be prepared from the two or more platelet concentrates without combining the two or more platelet concentrates. In some embodiments, the kit comprises instructions for using the processing set to prepare a plurality of platelet units from two or more apheresis platelet donations, wherein each of the platelet units is pathogen-inactivated and suitable for infusion into a subject, wherein the plurality of platelet units comprises a greater number of platelet units than can be prepared from the two or more apheresis platelet donations without combining the two or more apheresis platelet donations. In some embodiments, the PIC is a psoralen. In some embodiments, the PIC is amotosalen. In some embodiments, the platelet units in the plurality of platelet units each comprise a therapeutic dose of platelets.

In some embodiments, the kit comprises three or more third containers, each of which is coupled to the compound absorption device such that the two or more photochemically inactivated platelet donations can be transferred from the compound absorption device to the three or more third containers under sterile conditions to provide the plurality of pathogen-inactivated platelet units. In some embodiments, the kit comprises three third containers, each of which is coupled to the compound absorption device such that the two or more photochemically inactivated platelet donations can be transferred from the compound absorption device to the three third containers under sterile conditions to provide the plurality of pathogen-inactivated platelet units. In some embodiments, the first container is suitable for sterile coupling with an additional container. In some embodiments, the kit further comprises an additional container suitable for containing one or more of the two or more platelet donations, wherein the additional container is coupled to the first container such that the one or more platelet donations of the additional container can be transferred from the additional container to the first container under sterile conditions. In some embodiments, the additional container is suitable for containing a platelet concentrate. In some embodiments, the additional container is suitable for containing a platelet donation obtained by apheresis. In some embodiments, the additional container is suitable for sterile coupling with a second additional container. In some embodiments, the second additional container contains one or more of the two or more platelet donations, and wherein the additional container is suitable for sterile coupling with the second additional container such that the one or more platelet donations of the second additional container can be transferred from the second additional container to the additional container under sterile conditions.

In some embodiments, the kit further comprises a second additional container suitable for containing one or more of the two or more platelet donations, wherein the second additional container is coupled to the additional container such that the one or more platelet donations of the second additional container can be transferred from the second additional container to the additional container under sterile conditions. In some embodiments, the additional container is suitable for containing a platelet concentrate obtained from a first donor, and wherein the second additional container is suitable for containing a platelet concentrate obtained from a second donor. In some embodiments, the additional container is suitable for containing a platelet donation obtained by apheresis from a first donor, and wherein the second additional container is suitable for containing a platelet donation obtained by apheresis from a second donor.

It is to be understood that one, some, or all of the properties of the various embodiments described herein may be combined to form other embodiments. These and other aspects will become apparent to one of skill in the art. These and other embodiments are further described by the detailed description that follows.

### DESCRIPTION OF THE DRAWINGS

FIG. 1 shows a method of preparing three therapeutic units of platelets from two extra-large “single” apheresis platelet donations from a first donor and second donor. Abbreviations: XL, extra-large single; PI txt, pathogen inactivation treatment.

FIG. 2 shows a method of preparing five therapeutic units of platelets from two extra-large “double” apheresis platelet donations collected from a first donor and second donor. Abbreviations: XL, extra-large single; C, conventional single.

FIG. 3 shows a method of preparing four therapeutic units of platelets from one extra-large “double” apheresis platelet donation collected from a first donor and one extra-large “single” apheresis platelet donation collected from second donor. Abbreviations: XL, extra-large single; C, conventional single.

FIG. 4 shows a method of preparing seven therapeutic units of platelets from two extra-large “triple” apheresis platelet donations collected from a first donor and second donor. Abbreviations: XL, extra-large single; C, conventional single.

FIG. 5 shows a method of preparing six therapeutic units of platelets from one extra-large “triple” apheresis platelet donation collected from a first donor and one extra-large “double” apheresis platelet donation collected from second donor. Abbreviations: XL, extra-large single; C, conventional single.

FIG. 6 shows a method of preparing five therapeutic units of platelets from one extra-large “triple” apheresis platelet donation collected from a first donor and one extra-large “single” apheresis platelet donation collected from second donor. Abbreviations: XL, extra-large single; C, conventional single.

FIG. 7 shows an exemplary processing kit for use in preparing pathogen inactivated platelet units in accordance with some embodiments. Dotted components depict one container with platelets (e.g., donor platelets) sterile connected to the processing kit. Abbreviations: PIC, pathogen inactivating compound; CAD, compound adsorption device.

FIG. 8 shows an exemplary processing kit for use in preparing pathogen inactivated platelet units in accordance with some embodiments. Dotted components depict two containers with platelets (e.g., first donor platelets, second donor platelets) sterile connected to each other (e.g., for pooling) and to the processing kit. Abbreviations: PIC, pathogen inactivating compound; CAD, compound adsorption device.

FIG. 9 illustrates a system for optimization of platelet units for infusion.

### DETAILED DESCRIPTION

The term “pooled platelet preparation” refers to a preparation of platelets comprising platelets obtained from more than one donation, such as an apheresis platelet donation, and subsequently combined (e.g., in a single container). Generally, the platelet donations are obtained from different donors. Platelets may be pooled at any stage after donation and prior to therapeutic use, including but not limited to pooling before or after any addition of additive solution, before or after any storage period, and before or after any pathogen inactivation treatment or process. The platelets may be combined into any container suitable for platelet preparations and of sufficient size to accommodate the platelet volume, such as for example, by sterile connecting the containers of two platelet preparations (e.g., with connecting tubing) and transferring the platelets from one container into the other, or by sterile connecting the containers of two platelet preparations to a third container (e.g., with connecting tubing) and transferring the platelets into the third container.

The term “pathogen inactivation process” means a process useful to inactivate pathogens that may be present in a preparation of platelets, such as a platelet donation, where it is understood that the process does not necessarily inactivate completely all pathogens that may be present, but substantially reduces the amount of pathogens to significantly reduce the risk of a transfusion associated disease. The inactivation of a pathogen may be assayed by measuring the number of infective pathogen (e.g., viral or bacterial particles) in a certain volume, and the level of inactivation is typically represented in the log reduction in the infectivity of the pathogen, or log reduction in titer. Methods of assaying log reduction in titer, and measurements thereof for pathogen inactivation are known in the art. When the inactivation process is tested against a variety of pathogens, the reduction in a particular active pathogen is at least about 1 log, at least about 2 log, at least about 3 log, at least about 4 log, or at least about 5 log reduction in titer. A variety of pathogen inactivation processes are known in the art and may be used in the methods of the present disclosure, including for example, commercially available pathogen inactivation processes, such as INTERCEPT Blood System (Cerus Corp), Mirasol (TerumoBCT) and Theraflex (MacoPharma). In certain embodiments, a pathogen inactivation process may comprise treating with a pathogen inactivating compound.

The term “pathogen inactivating compound” means any suitable compound, such as a small organic compound, that can be used to inactivate a pathogen that may be present in a platelet-containing blood product. A “photoactivated pathogen inactivation compound” is a suitable compound that requires some level of light in order to sufficiently inactivate a pathogen. Such compounds are preferred in the inactivation of pathogens in platelet products as they provide control over the inactivation process. Such photoactivated pathogen inactivation compounds described herein include psoralens, isoalloxazines, alloxazines, phthalocyanines, phenothiazines, and porphyrins, where these terms are understood to encompass a general class of compounds, i.e. the core compound and suitable derivatives thereof. For example psoralens or a psoralen generally describes the psoralen core compound and any derivative thereof (e.g. amotosalen), isoalloxazines or an isoalloxazine generally describes the isoalloxazine core and any derivative thereof (e.g. riboflavin), and so forth. Such derivatives comprise the core compound structure as well as additional substituents on the core. Descriptions of such compounds include any salts thereof.

The term “amotosalen” means the compound 3-(2-aminoethoxymethyl)-2,5,9-trimethylfuro[3,2-g]chromen-7-one and any salts thereof. The compound may also be referred to as 4′-(4-amino-2-oxa)butyl-4,5′,8-trimethyl psoralen. The compound may also be referred to as 3-[(2-aminoethoxy) methyl]-2.5.9-trimethyl-7H-furo[3,2-g][1]benzopyran-7-one. Where the inactivation of platelets includes adding amotosalen HCl (the HCl salt of amotosalen) to a unit of platelets, the removal of this compound from the unit of platelets is not limited to the removal of amotosalen HCl, as the amotosalen can be present in solution as other salts or as the free base. As used in the methods described herein, removal of amotosalen means removal of the compound in any form, e.g. as the free base or as any salt, as measured by the assays described herein.

### Apheresis Collected Platelets

Certain aspects of the present disclosure provide systems and methods related to improvements (e.g., providing greater efficiency) in the collection and preparation of platelet units (e.g., apheresis-derived platelet units), including pathogen-inactivated platelet units, such as for example, in blood centers using apheresis collection systems for platelet donations. A particular benefit, among others, provided by the improvements disclosed herein is the opportunity to produce an increased number of platelet units (e.g., platelet units suitable for infusion) without increased the number of platelet donations or required donors. Apheresis generally refers to automated blood collection device that uses a centrifugal or filtration separation to automatically withdraw whole blood from a donor, separate the whole blood into blood components, collects certain of the components (e.g., platelets), and return to the donor the remainder of the whole blood and remaining uncollected blood components. Plateletpheresis is the collection of platelets using such an automated blood cell separator device, which results in obtaining a high yield of platelets (e.g., apheresis platelets) from a single donor. Some automated blood cell separator devices are capable of collection procedures not only for single platelet units, but also double and triple platelet units. Apheresis collection devices are well known in the art, with several such devices commercially available, including for example, the Amicus™ system (Fenwal, Inc), the Trima Accel® system (Terumo BCT) and the MCS®+9000 mobile system (Haemonetics, Inc).

Apheresis platelet donations are based on certain donor parameters, such as for example, gender, physical size (e.g., weight), hemoglobin level, platelet count on the day of donation, prior donation history and donation frequency, in part to ensure only a safe amount of platelets is collected. Any or all of these parameters may be entered into a computer system and/or the apheresis collection device. From these parameters, apheresis platelet donations generally are collected from an individual donor as a volume to yield one, two or three platelet units (e.g., therapeutic dosage units) each containing a specified minimum number (e.g., at least a specified minimum number) of platelets per unit to meet the therapeutic dose requirement, with such per unit or therapeutic dose criteria generally determined by governmental, regulatory or accrediting organization (e.g., industry) standards. Non-limiting examples of such standards include, for example, those set forth by FDA, EDQM, AABB, PMDA, TGA and SFDA. The specified minimum, for example, may vary by country. In some embodiments, platelet units may comprise at least about 2.0×10¹¹, at least about 2.2×10¹¹, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ or more platelets. Generally, a platelet number may be determined for each platelet unit, for example, based on a pre-donation platelet count and information about the volume collected, or alternatively by post-collection testing of units. Preferably, each platelet unit will comprise the specified minimum platelet number; however, determination of the platelet number for each unit may not be an absolute requirement and some platelet units in a plurality of platelet units may have less than a specified number.

The present disclosure may, in certain embodiments, refer in various ways to platelets collected from a donor (e.g., platelet donations, platelet preparations), such as for example as platelet donations or platelet preparations already collected (e.g., collected and stored), with or without further processing (e.g., leukofiltration, pathogen inactivation), platelet donations or platelet preparations currently being collected from a donor, and/or platelet donations or platelet preparations to be collected from a donor (e.g., a donor that has been identified, a present donor). Generally, reference to platelet donations or platelet preparations collected from a donor is prior to any pooling or combining step with additional platelets (e.g., from a different donor, such as a second donor or third donor) that may provide pooled platelet preparation.

In some embodiments of the present disclosure, platelets collected from a donor by apheresis may comprise less than the number (e.g., less than the minimum number, less than the minimum quantity, less than 100%) of platelets required to prepare one platelet unit (e.g., therapeutic dosage unit). For example, a platelet donation may comprise platelets in an amount that is less than about 99%, less than about 98%, less than about 97%, less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30% or less than about 25% of the number of platelets required to prepare one platelet unit. Such platelet donations may comprise a minimum amount of platelets, such as for example, at least about 10%, at least about 20%, at least about 30%, at least about 40% or at least about 50% or more of the number of platelets required to prepare one platelet unit. Alternatively or in addition, the platelet donation may comprise an amount of platelets that is about 10% to about 98%, about 10% to about 95%, about 10% to about 90%, 15% to about 98%, about 15% to about 95%, about 15% to about 90%, 20% to about 98%, about 20% to about 95%, about 20% to about 90%, 25% to about 98%, about 25% to about 95%, about 25% to about 90%, 30% to about 98%, about 30% to about 95%, about 30% to about 90%, 35% to about 98%, about 35% to about 95%, about 35% to about 90%, 40% to about 98%, about 40% to about 95%, about 40% to about 90%, 50% to about 98%, about 50% to about 95% or about 50% to about 90% of the number of platelets required to prepare one platelet unit. In some embodiments, the platelet donation comprises an amount of platelets that is about 98%, about 95%, about 90%, about 85%, about 80%, about 75%, about 70%, about 65%, about 60%, about 55%, about 50%, about 45%, about 40%, about 35%, about 30% or about 25% of the number of platelets required to prepare one platelet unit.

In some embodiments, platelets collected from a donor by apheresis may comprise greater than the number (e.g., greater than the minimum number, greater than the minimum quantity) of platelets required to prepare one platelet unit, but less than the number (e.g., less than the minimum number, less than the minimum quantity) of platelets required to prepare two platelet units. For example, a platelet donation comprising greater than the number of platelets required to prepare one platelet unit, but less than the number of platelets required to prepare two platelet units may comprise platelets in an amount that is about 1.1-fold, about 1.15-fold, about 1.2-fold, about 1.25-fold, about 1.3-fold, about 1.35-fold, about 1.4-fold, about 1.45-fold, about 1.5-fold, about 1.6-fold, about 1.7-fold, about 1.8-fold, about 1.9-fold, about 1.95-fold or about 1.98-fold the number of platelets required to prepare one platelet unit. Alternatively or in addition, a platelet donation comprising greater than the number of platelets required to prepare one platelet unit, but less than the number of platelets required to prepare two platelet units may comprise platelets in an amount that is about 1.1-fold to about 1.98-fold, about 1.1-fold to about 1.95-fold, about 1.1-fold to about 1.9-fold, about 1.15-fold to about 1.98-fold, about 1.15-fold to about 1.95-fold, about 1.15-fold to about 1.9-fold, about 1.2-fold to about 1.98-fold, about 1.2-fold to about 1.95-fold, about 1.2-fold to about 1.9-fold, about 1.25-fold to about 1.98-fold, about 1.25-fold to about 1.95-fold, about 1.25-fold to about 1.9-fold, about 1.3-fold to about 1.98-fold, about 1.3-fold to about 1.95-fold, about 1.3-fold to about 1.9-fold, about 1.35-fold to about 1.98-fold, about 1.35-fold to about 1.95-fold, about 1.35-fold to about 1.9-fold, about 1.4-fold to about 1.98-fold, about 1.4-fold to about 1.95-fold, about 1.4-fold to about 1.9-fold, about 1.5-fold to about 1.98-fold, about 1.5-fold to about 1.95-fold, or about 1.5-fold to about 1.9-fold the number of platelets required to prepare one platelet unit. Alternatively or in addition, a platelet donation comprising greater than the number of platelets required to prepare one platelet unit, but less than the number of platelets required to prepare two platelet units may comprise platelets in an amount that is about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195% or about 198% the number of platelets required to prepare one platelet unit. Alternatively or in addition, a platelet donation comprising greater than the number of platelets required to prepare one platelet unit, but less than the number of platelets required to prepare two platelet units may comprise platelets in an amount that is about 110% to about 198%, about 110% to about 195%, about 110% to about 190%, about 115% to about 198%, about 115% to about 195%, about 115% to about 190%, about 120% to about 198%, about 120% to about 195%, about 120% to about 190%, about 125% to about 198%, about 125% to about 195%, about 125% to about 190%, about 130% to about 198%, about 130% to about 195%, about 130% to about 190%, about 135% to about 198%, about 135% to about 195%, about 135% to about 190%, about 140% to about 198%, about 140% to about 195%, about 140% to about 190%, about 150% to about 198%, about 150% to about 195%, or about 150% to about 190% the number of platelets required to prepare one platelet unit.

In some embodiments, platelets collected from a donor by apheresis may comprise greater than the number (e.g., greater than the minimum number, greater than the minimum quantity) of platelets required to prepare two platelet units, but less than the number (e.g., less than the minimum number, less than the minimum quantity) of platelets required to prepare three platelet units. For example, a platelet donation comprising greater than the number of platelets required to prepare two platelet units, but less than the number of platelets required to prepare three platelet units may comprise platelets in an amount that is about 1.1-fold, about 1.15-fold, about 1.2-fold, about 1.25-fold, about 1.3-fold, about 1.35-fold, about 1.4-fold, about 1.45-fold, about 1.5-fold, about 1.6-fold, about 1.7-fold, about 1.8-fold, about 1.9-fold, about 1.95-fold or about 1.98-fold the number of platelets required to prepare two platelet units. Alternatively or in addition, a platelet donation comprising greater than the number of platelets required to prepare two platelet units, but less than the number of platelets required to prepare three platelet units may comprise platelets in an amount that is about 1.1-fold to about 1.98-fold, about 1.1-fold to about 1.95-fold, about 1.1-fold to about 1.9-fold, about 1.15-fold to about 1.98-fold, about 1.15-fold to about 1.95-fold, about 1.15-fold to about 1.9-fold, about 1.2-fold to about 1.98-fold, about 1.2-fold to about 1.95-fold, about 1.2-fold to about 1.9-fold, about 1.25-fold to about 1.98-fold, about 1.25-fold to about 1.95-fold, about 1.25-fold to about 1.9-fold, about 1.3-fold to about 1.98-fold, about 1.3-fold to about 1.95-fold, about 1.3-fold to about 1.9-fold, about 1.35-fold to about 1.98-fold, about 1.35-fold to about 1.95-fold, about 1.35-fold to about 1.9-fold, about 1.4-fold to about 1.98-fold, about 1.4-fold to about 1.95-fold, about 1.4-fold to about 1.9-fold, about 1.5-fold to about 1.98-fold, about 1.5-fold to about 1.95-fold, or about 1.5-fold to about 1.9-fold the number of platelets required to prepare two platelet units. Alternatively or in addition, a platelet donation comprising greater than the number of platelets required to prepare two platelet units, but less than the number of platelets required to prepare three platelet units may comprise platelets in an amount that is about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195% or about 198% the number of platelets required to prepare two platelet units. Alternatively or in addition, a platelet donation comprising greater than the number of platelets required to prepare two platelet units, but less than the number of platelets required to prepare three platelet units may comprise platelets in an amount that is about 110% to about 198%, about 110% to about 195%, about 110% to about 190%, about 115% to about 198%, about 115% to about 195%, about 115% to about 190%, about 120% to about 198%, about 120% to about 195%, about 120% to about 190%, about 125% to about 198%, about 125% to about 195%, about 125% to about 190%, about 130% to about 198%, about 130% to about 195%, about 130% to about 190%, about 135% to about 198%, about 135% to about 195%, about 135% to about 190%, about 140% to about 198%, about 140% to about 195%, about 140% to about 190%, about 150% to about 198%, about 150% to about 195%, or about 150% to about 190% the number of platelets required to prepare two platelet units.

In some embodiments, platelets collected from a donor by apheresis may comprise greater than the number (e.g., greater than the minimum number, greater than the minimum quantity) of platelets required to prepare three platelet units, but less than the number (e.g., less than the minimum number, less than the minimum quantity) of platelets required to prepare four platelet units. For example, a platelet donation comprising greater than the number of platelets required to prepare three platelet units, but less than the number of platelets required to prepare four platelet units may comprise platelets in an amount that is about 1.1-fold, about 1.15-fold, about 1.2-fold, about 1.25-fold, about 1.3-fold, about 1.35-fold, about 1.4-fold, about 1.45-fold, about 1.5-fold, about 1.6-fold, about 1.7-fold, about 1.8-fold, about 1.9-fold, about 1.95-fold or about 1.98-fold the number of platelets required to prepare three platelet units. Alternatively or in addition, a platelet donation comprising greater than the number of platelets required to prepare three platelet units, but less than the number of platelets required to prepare four platelet units may comprise platelets in an amount that is about 1.1-fold to about 1.98-fold, about 1.1-fold to about 1.95-fold, about 1.1-fold to about 1.9-fold, about 1.15-fold to about 1.98-fold, about 1.15-fold to about 1.95-fold, about 1.15-fold to about 1.9-fold, about 1.2-fold to about 1.98-fold, about 1.2-fold to about 1.95-fold, about 1.2-fold to about 1.9-fold, about 1.25-fold to about 1.98-fold, about 1.25-fold to about 1.95-fold, about 1.25-fold to about 1.9-fold, about 1.3-fold to about 1.98-fold, about 1.3-fold to about 1.95-fold, about 1.3-fold to about 1.9-fold, about 1.35-fold to about 1.98-fold, about 1.35-fold to about 1.95-fold, about 1.35-fold to about 1.9-fold, about 1.4-fold to about 1.98-fold, about 1.4-fold to about 1.95-fold, about 1.4-fold to about 1.9-fold, about 1.5-fold to about 1.98-fold, about 1.5-fold to about 1.95-fold, or about 1.5-fold to about 1.9-fold the number of platelets required to prepare three platelet units. Alternatively or in addition, a platelet donation comprising greater than the number of platelets required to prepare three platelet units, but less than the number of platelets required to prepare four platelet units may comprise platelets in an amount that is about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195% or about 198% the number of platelets required to prepare three platelet units. Alternatively or in addition, a platelet donation comprising greater than the number of platelets required to prepare three platelet units, but less than the number of platelets required to prepare four platelet units may comprise platelets in an amount that is about 110% to about 198%, about 110% to about 195%, about 110% to about 190%, about 115% to about 198%, about 115% to about 195%, about 115% to about 190%, about 120% to about 198%, about 120% to about 195%, about 120% to about 190%, about 125% to about 198%, about 125% to about 195%, about 125% to about 190%, about 130% to about 198%, about 130% to about 195%, about 130% to about 190%, about 135% to about 198%, about 135% to about 195%, about 135% to about 190%, about 140% to about 198%, about 140% to about 195%, about 140% to about 190%, about 150% to about 198%, about 150% to about 195%, or about 150% to about 190% the number of platelets required to prepare three platelet units.

In some or all of the aforementioned embodiments, when a donation comprises greater than the number of platelets required to prepare a particular (e.g., targeted, desired) number of platelet units (e.g., one platelet unit, two platelet units, three platelet units), greater than the number of platelets required to prepare the particular number of units refers to the donation comprising a number of platelets greater than the number required to prepare the particular number of units after factoring in any platelet losses from processing, sampling (e.g., sampling for testing) and/or pathogen inactivation treatment. Likewise, in some or all of the aforementioned embodiments, when a donation comprises less than the number of platelets required to prepare a particular (e.g., targeted, desired) number of platelet units (e.g., one platelet unit, two platelet units, three platelet units), less than the number of platelets required to prepare the particular number of units refers to the donation comprising a number of platelets less than the number required to prepare the particular number of units after factoring in any platelet losses from processing, sampling (e.g., sampling for testing) and/or pathogen inactivation treatment. In some embodiments, a donation may comprise more than the number of platelets required to prepare a particular number of platelet units (e.g., one platelet unit, two platelet units, three platelet units) prior to any processing, sampling and/or pathogen inactivation treatment, but less than the number of platelets required to prepare the same number of platelet units after any processing, sampling and/or pathogen inactivation treatment.

### Preparation of Platelet Units

In certain aspects, the present disclosure provides methods generally related to preparing multiple units of platelets, such as for example, a plurality of platelet units or two or more therapeutic dosage units of platelets. For example, in one aspect, the present disclosure provides a method of preparing a plurality of platelet units suitable for infusion, comprising: a) collecting a first platelet donation by apheresis from a first donor, b) collecting a second platelet donation by apheresis from a second donor, c) combining the first platelet donation and the second platelet donation to yield a pooled platelet preparation, and d) separating the pooled platelet preparation into a plurality of platelet units (e.g., therapeutic doses, therapeutic dosage units) each in an individual container, wherein the plurality of platelet units comprises a greater number of platelet units than the total number of platelet units that can be prepared from the first platelet donation and second platelet donation without combining the first and second platelet donations.

In another aspect, the present disclosure also provides a method of preparing a plurality of platelet units suitable for infusion, comprising: a) maintaining an inventory log of an inventory of apheresis-collected platelet preparations, the inventory log comprising for each platelet preparation a unique identifier, a donor blood type, a donation time and a quantity of platelets in the preparation, b) identifying an available donor suitable for donating platelets by apheresis, c) selecting from the inventory log at least one platelet preparation in the inventory for combining with platelets collected from the available donor, d) collecting a new platelet donation by apheresis from the available donor, e) combining the at least one selected platelet preparation and the new platelet donation to yield a pooled platelet preparation, and f) separating the pooled platelet preparation into a plurality of platelet units each in an individual container, wherein the plurality of platelet units comprises a greater number of platelet units than the total number of platelet units that can be prepared from the at least one selected platelet preparation and the new platelet donation without combining the at least one selected platelet preparation and new platelet donation.

In another aspect, the present disclosure further provides a method of preparing two or more therapeutic dosage units of platelets comprising: a) combining two or more apheresis-derived platelet donor units to form a pooled platelet composition, b) subjecting the pooled platelet composition to a pathogen inactivation process, and c) separating the pooled platelet composition into two or more therapeutic dosage units of platelets, each in an individual container. In some embodiments, the number of therapeutic dosage units of platelets following step c) is greater than the number of therapeutic dosage units of platelets that can be prepared from the two or more apheresis-derived platelet donor units individually, without combining the two or more donor units.

In another aspect, the present disclosure also provides, a method for collecting a platelet donation by apheresis from a donor, comprising: a) maintaining an inventory log of an inventory of apheresis-collected platelet preparations, the inventory log comprising for each platelet preparation a unique identifier, a donor blood type, a donation date and a quantity of platelets in the preparation, b) identifying a donor suitable for donating platelets by apheresis, c) selecting from the inventory log one or more platelet preparations suitable for combining with a new platelet donation to be collected by apheresis from the donor, d) determining a target volume of the new platelet donation, wherein the target volume optimizes the number of platelet units that can be prepared from a combination comprising the new platelet donation and the one or more selected platelet preparations; and e) collecting the target volume of the new platelet donation by apheresis from the donor. In some embodiments, the method further comprises: f) combining the one or more selected platelet preparations and the new platelet donation to yield a pooled platelet preparation; and g) separating the pooled platelet preparation into a plurality of platelet units (e.g., therapeutic dosage units) each in an individual container. In some embodiments, the plurality of platelet units comprises a greater number of platelet units than the total number of platelet units that can be prepared from the one or more selected platelet preparations and the new platelet donation without combining the one or more selected platelet preparations and the new platelet donation.

In another aspect, the present disclosure also provides a computer-implemented method for optimization of platelet units (e.g., platelet units for infusion), comprising: at a computer system including one or more processors and memory, receiving one or more values associated with one or more properties of a platelet donation; instructing to search, based on the received one or more values, a plurality of values associated with representations of a plurality of platelet preparations; determining, based on the search results, a volume of the platelet donation; and providing the determined volume of the platelet donation, wherein the determined volume of the platelet donation enables optimization of platelet units (e.g., platelet units for infusion). In some embodiments, optimization of platelet units comprises optimizing the number of platelet units that can be prepared (e.g., from the platelet donation and the plurality of platelet preparations, relative to the process for preparing platelet units before optimizing). In some embodiments, optimization of platelet units comprises reducing excess platelet platelets per platelet unit (e.g., relative to the process for preparing platelet units before optimizing). In some embodiments, optimization of platelet units comprises reducing or eliminating platelet waste (e.g., relative to the process for preparing platelet units before optimizing). In some embodiments, optimization of platelet units comprises improving or increasing the number of platelet units prepared (e.g., from the platelet donation and the plurality of platelet preparations, relative to the process for preparing platelet units before optimizing). In some embodiments, optimization of platelet units comprises increasing the efficiency of preparing platelet units (e.g., relative to the process for preparing platelet units before optimizing). In some embodiments, optimization of platelet units comprises obtaining a target value (e.g., number) of platelets per platelet unit. In some embodiments, at least one of the platelet donation and the plurality of platelet preparations comprises apheresis-derived platelets. In some embodiments, the platelet donation comprises platelets being currently collected from a donor or platelets to be collected from a donor. In some embodiments, the one or more properties of the platelet donation comprises: a blood type, a donation time (e.g., date, time, date and time), number of platelets per unit volume, and optionally a maximum platelet donation volume. In some embodiments, the value of the blood type includes one of: A, B, AB, or O.

In some embodiments, optimization of platelet units can be performed using quantitative approaches. For example, using a quantitative approach, searching of the plurality of values associated with representations of a plurality of platelet preparations may be performed with respect to the stored data of platelet preparations. In some examples, the stored data of platelet preparations may be dynamic such that they are continuously varying as new donors donate (e.g., new platelet donations are available) and platelet preparations (e.g., older platelet preparations) are no longer available or of suitable for use in optimization of platelet units (e.g., have been used for infusion, have become expired). In some embodiments, optimization of platelet units is performed with each platelet donation (e.g., new platelet donation). In some embodiments, optimization of platelet units is performed at time intervals, such as for example, 1 hour, 2 hour, 3 hour, 4 hour, 5 hour, 6 hour, 7 hour, 8 hour, 9 hour, 10 hour, 12 hour, 14 hour, 16 hour, 18 hour, 20 hour, 24 hour, 36 hour, 48 hour, 60 hour, 72 hour or 96 hour intervals. In some embodiments, the quantitative approach can utilize regression and/or time series analysis for predicting or forecasting the available platelet preparations for optimizing the platelet units. Examples of the time series analysis include autoregressive integrated moving average, the Holt-Winters family of exponential smoothing models, neural-network based models, or the like.

In some embodiments, optimization of platelet units can be performed using qualitative approaches. For example, using a qualitative approach, searching of the plurality of values associated with representations of a plurality of platelet preparations may be performed using expert judgment or management assumptions, with or without entire actual data of platelet preparations. A qualitative approach enables the optimization of the platelet units under the situation where the entire record of platelet preparation may not readily available, or may be only partially available. The assumptions may be made using empirical data of the platelet preparations and/or linear or non-linear regression models. Various models that may be used to optimize platelet units include, for example, exponential smoothing model (exponentially decreases weights of past data), auto-regressive, moving average, autoregressive-moving average, or Box-Jenkins forecasting models (uses a variable's past behavior to select the best forecasting model).

In some embodiments, instructing to search the plurality of values associated with representations of a plurality of platelet preparations comprises: obtaining one or more criteria for searching using the one or more received values associated with one or more properties of the platelet donation; instructing to search the plurality of values associated with the representations of the plurality of platelet preparations based on the one or more criteria for searching; and obtaining one or more candidate representations of platelet preparations from the representations of the plurality of platelet preparations, the one or more candidate representations of platelet preparations having values that satisfy the one or more criteria for searching. In some embodiments, the criteria for searching include one or more threshold conditions of one or more of: a blood type, a donation time, a platelet volume and number of platelets per unit volume. In some embodiments, obtaining the one or more candidate representations of platelet preparations comprises filtering the representations of the plurality of platelet preparations based on the one or more criteria for searching. In some embodiments, the representations of the plurality of platelet preparations include at least one of: platelet preparations stored in a platelet preparation inventory, platelet preparations being currently collected from a plurality of donors, and platelet preparations to be collected from a plurality of donors.

Various searching algorithms may be used for searching a plurality of values associated with representations of a plurality of platelet preparations. A search algorithm retrieves information stored with a data structure such as linked lists, arrays, search trees, hash tables, or the like. A search algorithm may include one or more of an exhaustive search algorithm, a heuristic based searching algorithm, simulated annealing, Tabu search, A-teams, genetic programming, a tree searching algorithm (depth-first search, breadth-first search), combinatorial search (minimax algorithm, alpha-beta pruning), graph search algorithm (Dijkstra's algorithm, Kruskal's algorithm, nearest neighbor algorithm, Prim's algorithm), or the like. It is appreciated that any desired algorithm can be used to perform the search of a plurality of values associated with representations of a plurality of platelet preparations.

In some embodiments, determining the volume of the platelet donation comprises: obtaining one or more values corresponding to one or more candidate representations of platelet preparations provided by the search results; obtaining one or more criteria for platelet units, the criteria for platelet units enabling optimization of platelet units; determining, based on the one or more values corresponding to one or more candidate representations of platelet preparations and the one or more criteria for platelet units, one or more platelet preparations to select from the one or more candidate representations of the plurality of platelet preparations; and obtaining the volume of the platelet donation based on the determined platelet preparation. In some embodiments, providing the determined volume of the platelet donation comprises at least one of: displaying the determined volume of the platelet donation on a display of the computer system; sending a notification providing the determining volume of the platelet donation; and providing the determined volume of the platelet donation to an apheresis system. In some embodiments, the method further comprises combining the platelet donation and one or more platelet preparations selected from the representations of the plurality of platelet preparations to yield a pooled platelet preparation. In some embodiments, the method further comprises separating the pooled platelet preparation into a plurality of platelet units (e.g., therapeutic dosage units of platelets) each in an individual container.

In some embodiments, to determine one or more platelet preparations, various sorting algorithm can be used. In some examples, the sorting algorithm may include one or more of insertion sort, selection sort, merge sort, heapsort, quicksort, bubble sort, shellsort, comb sort, counting sort, bucket sort, radix sort, timsort, cubesort, binary tree sort, cycle sort, patience sorting, strand sort, or the like. The sorting may be based on the search results that include one or more candidate representations of platelet preparations and based on the one or more criteria for platelet units. The one or more criteria can include, for example, threshold conditions of one or more of: a blood type, a donation time, a volume, and number of platelets per unit volume; and can enable optimization of platelet units. For example, the criteria may enable reducing waste of platelet units. The sorting results can identify one or more platelet preparations for obtaining the volume of the platelet donation. For example, the sorting results may identify a top list of platelet preparations that satisfy the criteria with an acceptable confidence level (e.g., confidence level above a predetermined threshold). The volume of the platelet donation can thus be determined based on these platelet preparations identified by the sorting results.

In yet another aspect, the present disclosure provides a computer-implemented method for optimization of platelet units (e.g., platelet units for infusion), comprising: at a computer system including one or more processors and memory, receiving one or more values associated with one or more properties of a first platelet preparation; instructing to search, based on the received one or more values, a plurality of values associated with representations of a plurality of platelet preparations; selecting, based on the search results, a second platelet preparation from the representations of a plurality of platelet preparations; and providing the selected second platelet preparation, wherein the selected second platelet preparation enables optimization of platelet units (e.g., platelet units for infusion). In some embodiments, optimization of platelet units comprises optimizing the number of platelet units that can be prepared (e.g., from the platelet donation and the plurality of platelet preparations, relative to the process for preparing platelet units before optimizing). In some embodiments, optimization of platelet units comprises reducing excess platelet platelets per platelet unit (e.g., relative to the process for preparing platelet units before optimizing). In some embodiments, optimization of platelet units comprises reducing or eliminating platelet waste (e.g., relative to the process for preparing platelet units before optimizing). In some embodiments, optimization of platelet units comprises improving or increasing the number of platelet units prepared (e.g., from the platelet donation and the plurality of platelet preparations, relative to the process for preparing platelet units before optimizing). In some embodiments, optimization of platelet units comprises increasing the efficiency of preparing platelet units (e.g., relative to the process for preparing platelet units before optimizing). In some embodiments, optimization of platelet units comprises obtaining a target value (e.g., number) of platelets per platelet unit. In some embodiments, at least one of the first platelet preparation and the plurality of platelet preparations comprises apheresis-derived platelets. In some embodiments, the first platelet preparation comprises a platelet preparation stored in a platelet preparation inventory. In some embodiments, the one or more properties of the first platelet preparation comprises: a blood type, a donation time (e.g., date, time, date and time), a volume, and number of platelets per unit volume. In some embodiments, the value of the blood type includes one of: A, B, AB, or O.

In some embodiments, instructing to search the plurality of values associated with representations of a plurality of platelet preparations comprises: obtaining one or more criteria for searching using the one or more received values associated with one or more properties of the first platelet preparation; instructing to search the plurality of values associated with the representations of the plurality of platelet preparations based on the one or more criteria for searching; and obtaining one or more candidate representations of platelet preparations from the representations of the plurality of platelet preparations, the one or more candidate representations of platelet preparations having values that satisfy the one or more criteria for searching. In some embodiments, the criteria for searching include one or more threshold conditions of one or more of: a blood type, a donation time, a volume, and number of platelets per unit volume. In some embodiments, obtaining the one or more candidate representations of platelet preparations comprises filtering the representations of the plurality of platelet preparations based on the one or more criteria for searching. In some embodiments, the representations of the plurality of platelet preparations include at least one of: platelet preparations stored in a platelet preparation inventory, platelet preparations being currently collected from a plurality of donors, and platelet preparations to be collected from a plurality of donors.

In some embodiments, selecting the second platelet preparation comprises: obtaining one or more values corresponding to one or more candidate representations of platelet preparations provided by the search results; obtaining one or more criteria for platelet units, the criteria for platelet units enabling optimization of platelet units; and selecting, based on the one or more values corresponding to one or more candidate representations of platelet preparations and the one or more criteria for platelet units, a second platelet preparation from the one or more candidate representations of platelet preparations. In some embodiments, providing the selected second platelet preparation comprises at least one of: displaying the selected second platelet preparation on a display of the computer system; and sending a notification providing the selected second platelet preparation; and providing an identification of the selected second platelet preparation to an apheresis system. In some embodiments, the method further comprises combining the first platelet preparation and selected second platelet preparation to yield a pooled platelet preparation. In some embodiments, the method further comprises separating the pooled platelet preparation into a plurality of platelet units (e.g., therapeutic dosage units of platelets) each in an individual container.

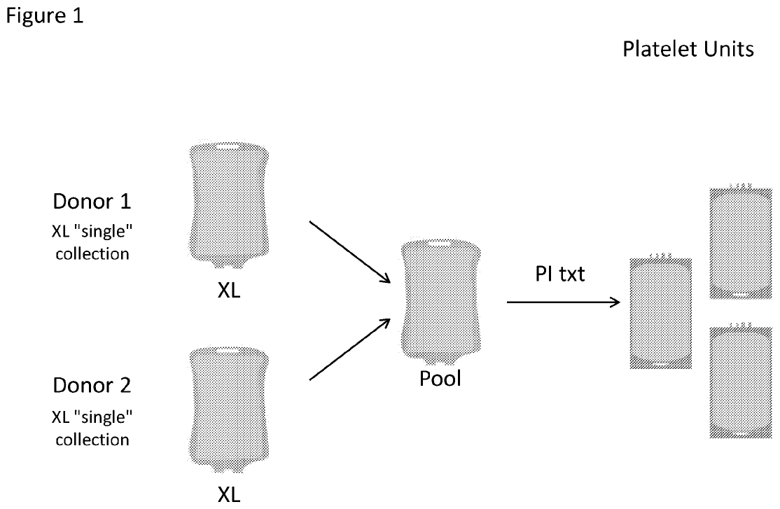

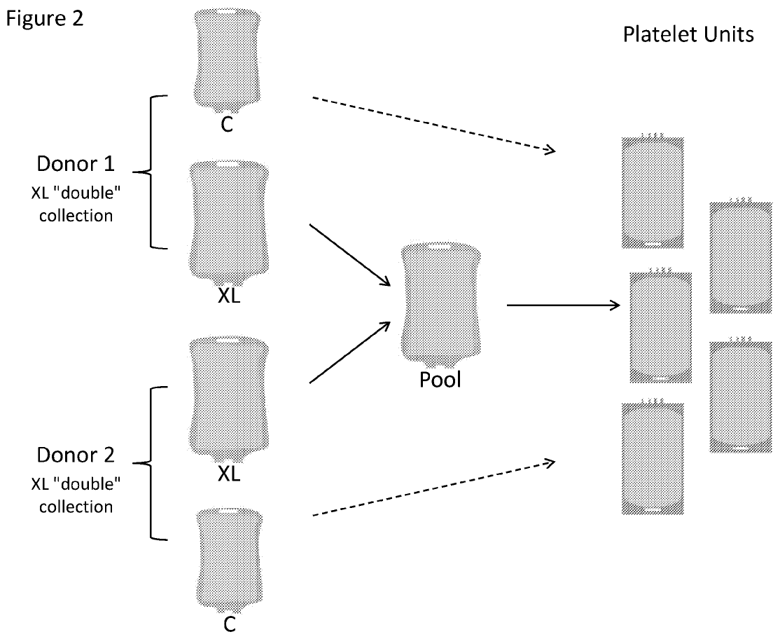

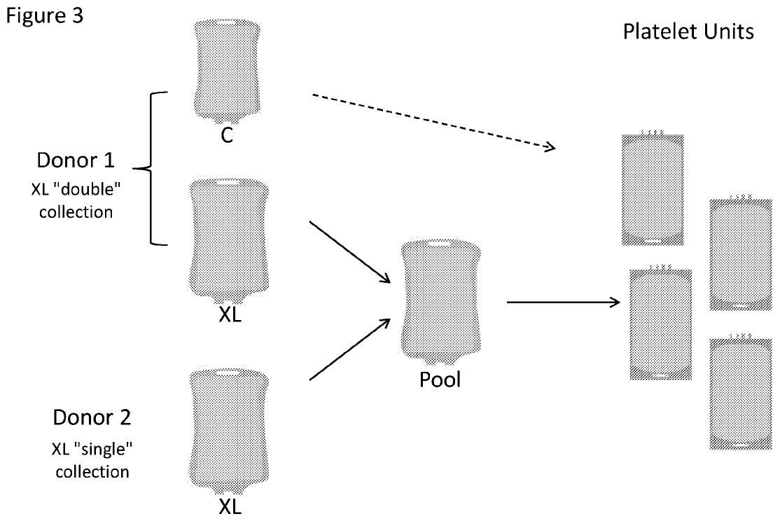

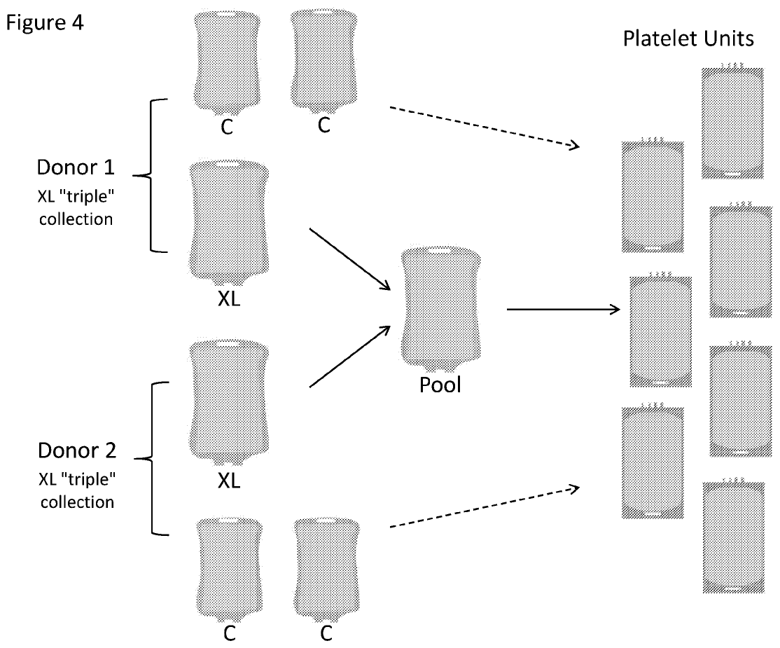

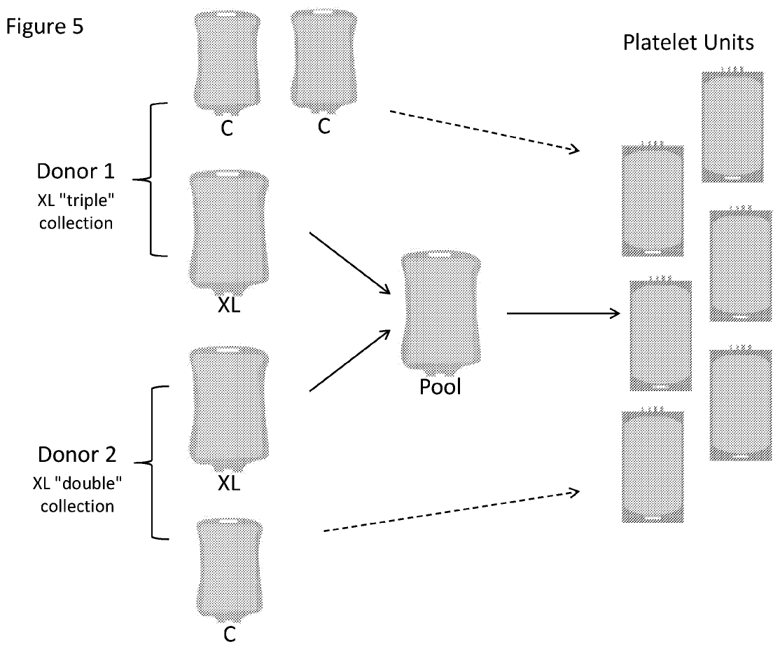

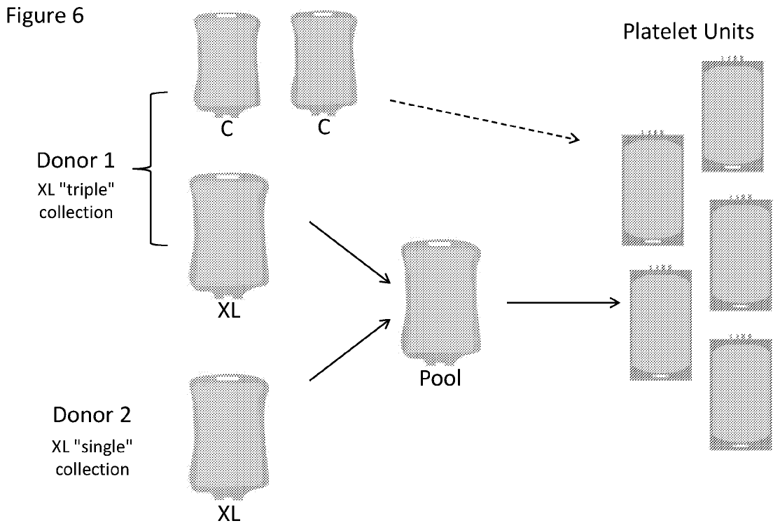

In any or all of the aforementioned aspects and/or embodiments, when two or more platelet donations/platelet preparations are combined to yield a pooled platelet preparation, each of the platelet donations/platelet preparations may comprise an amount (e.g., number) of platelets that is similar or different from the other, such as set forth above. For example, one of the platelet donations (e.g., a first platelet donation) may comprise less than the number of platelets required to prepare one platelet unit and the other platelet donation (e.g., a second platelet donation) may comprise greater than the number of platelets required to prepare one platelet unit, but less than the number of platelets required to prepare two platelet units. Alternatively, one of the platelet donations may comprise less than the number of platelets required to prepare one platelet unit and the second platelet donation may comprise greater than the number of platelets required to prepare two platelet units, but less than the number of platelets required to prepare three platelet units. Alternatively, one of the platelet donations may comprise less than the number of platelets required to prepare one platelet unit and the other platelet donation may comprise greater than the number of platelets required to prepare three platelet units, but less than the number of platelets required to prepare four platelet units. Alternatively, one of the platelet donations may comprise greater than the number of platelets required to prepare one platelet unit, but less than the number of platelets required to prepare two platelet units, and the other platelet donation may comprise greater than the number of platelets required to prepare one platelet unit, but less than the number of platelets required to prepare two platelet units (see e.g., FIG. 1). Alternatively, one of the platelet donations may comprise greater than the number of platelets required to prepare one platelet unit, but less than the number of platelets required to prepare two platelet units, and the other platelet donation may comprise greater than the number of platelets required to prepare two platelet units, but less than the number of platelets required to prepare three platelet units (see e.g., FIG. 3). Alternatively, one of the platelet donations may comprise greater than the number of platelets required to prepare one platelet unit, but less than the number of platelets required to prepare two platelet units, and the other platelet donation may comprise greater than the number of platelets required to prepare three platelet units, but less than the number of platelets required to prepare four platelet units (see e.g., FIG. 6). Alternatively, one of the platelet donations may comprise greater than the number of platelets required to prepare two platelet units, but less than the number of platelets required to prepare three platelet units, and the other platelet donation may comprise greater than the number of platelets required to prepare two platelet units, but less than the number of platelets required to prepare three platelet units (see e.g., FIG. 2). Alternatively, one of the platelet donations may comprise greater than the number of platelets required to prepare two platelet units, but less than the number of platelets required to prepare three platelet units, and the other platelet donation may comprise greater than the number of platelets required to prepare three platelet units, but less than the number of platelets required to prepare four platelet units (see e.g., FIG. 5). Alternatively, one of the platelet donations may comprise greater than the number of platelets required to prepare three platelet units, but less than the number of platelets required to prepare four platelet units, and the other platelet donation may comprise greater than the number of platelets required to prepare three platelet units, but less than the number of platelets required to prepare four platelet units (see e.g., FIG. 4).

The present disclosure also contemplates that in any or all aspects and/or embodiments, a pooled platelet preparation may comprise more than two platelet donations/platelet preparations (e.g., a third platelet donation, a second new platelet donation). For example, in some embodiments, the method further comprises collecting a third platelet donation by apheresis from a third donor, and combining the third platelet donation with a first platelet donation and a second platelet donations to yield a pooled platelet preparation, and separating the pooled platelet preparation into a plurality of platelet units each in an individual container, wherein the plurality of platelet units comprises a greater number of platelet units than the total number of platelet units that can be prepared from the first platelet donation, the second platelet donation and the third platelet donation without combining the first, second and third platelet donations. In some embodiments, the third platelet donation comprises greater than the number of platelets required to prepare one platelet unit, but less than the number of platelets required to prepare two platelet units. In some embodiments, the third platelet donation comprises greater than the number of platelets required to prepare two platelet units, but less than the number of platelets required to prepare three platelet units. In some embodiments, the third platelet donation comprises greater than the number of platelets required to prepare three platelet units, but less than the number of platelets required to prepare four platelet units.

In some embodiments, the method further comprises identifying a second available donor suitable for donating platelets by apheresis, collecting a second new platelet donation by apheresis from the second available donor, combining the second new platelet donation with an at least one selected platelet preparation and a new platelet donation to yield the pooled platelet preparation, and separating the pooled platelet preparation into a plurality of platelet units each in an individual container, wherein the plurality of platelet units comprises a greater number of platelet units than the total number of platelet units that can be prepared from the at least one selected platelet preparation, the new platelet donation and the second new platelet donation without combining the at least one selected platelet preparation, the new platelet donation and the second new platelet donation.

In some embodiments, the method further comprises collecting a third platelet donation by apheresis from a third donor, combining the third platelet donation with an at least one selected platelet preparation and a new platelet donation to yield a pooled platelet preparation, and separating the pooled platelet preparation into a plurality of platelet units each in an individual container, wherein the plurality of platelet units comprises a greater number of platelet units than the total number of platelet units that can be prepared from the at least one selected platelet preparation, the new platelet donation and the third platelet donation without combining the at least one selected platelet preparation, the new platelet donation and the third platelet donation. In some embodiments, the method further comprises subjecting the at least one selected platelet preparation, the new platelet donation, and/or the third platelet donation to a pathogen inactivation process.

In some of the aforementioned embodiments, the pooled platelet preparation comprises platelets only from donors (e.g., first donor, second donor, third donor) of the same ABO blood type. In some embodiments, the pooled platelet preparation comprises platelets only from donors (e.g., first donor, second donor, third donor) of the same ABO and Rh type.

Generally, when pooling two or more platelet donations and/or platelet preparations, it is preferred that the platelet donations and/or platelet preparations are derived from donations relatively close in time, such as for example within about 24 hours, within about 18 hours, within about 12 hours, or within about 6 hours of each other. Thus, in certain embodiments, the time of donation (e.g., date, time, date and time) may be information of importance to be captured and/or associated with the platelet donation and/or platelet preparation. The time of donation may, in certain embodiments, refer to the time of initiating apheresis collection of platelets and/or time of completing apheresis collection of platelets. Additionally, a donation time may be, for example, a precise time (e.g., to the nearest minute) or a time rounded to a time increment, such as the nearest 15 minute increment or 1 hour increment, or a time within a time range, such as 15 minute interval or time period or 1 hour interval or time period. In some of the aforementioned embodiments, platelet donations and/or platelet preparations are collected less than about (e.g., within) 24 hours, less than about 22 hours, less than about 20 hours, less than about 16 hours, less than about 12 hours, less than about 10 hours, less than about 8 hours, less than about 6 hours, less than about 5 hours, less than about 4 hours, less than about 3 hours, less than about 2 hours or less than about 1 hour apart. In some embodiments, the second platelet donation is collected about the same time (e.g., overlapping collection time) as the first platelet donation. In some embodiments, platelet donations and/or platelet preparations are collected at about the same time (e.g., overlapping collection time). In some embodiments, platelet donations and/or platelet preparations are collected within a period (e.g., the same period) of about 24 hours, about 22 hours, about 20 hours, about 16 hours, about 12 hours, about 10 hours, about 8 hours, about 6 hours, about 5 hours, about 4 hours, about 3 hours, about 2 hours or about 1 hour.

The pooled platelet preparation in any of the aforementioned embodiments may comprise, for example, at least about 5.0×10¹¹, at least about 5.5×10¹¹, at least about 6.0×10¹¹, at least about 6.5×10¹¹, at least about 7.0×10¹¹, at least about 7.5×10¹¹, at least about 8.0×10¹¹, at least about 8.5×10¹¹, at least about 9.0×10¹¹, at least about 9.5×10¹¹, at least about 10.0×10¹¹, at least about 10.5×10¹¹, at least about 11.0×10¹¹, at least about 11.5×10¹¹, at least about 12.0×10¹¹, at least about 12.5×10¹¹, at least about 13.0×10¹¹, at least about 13.5×10¹¹, at least about 14.0×10¹¹, at least about 14.5×10¹¹, or at least about 15.0×10¹¹ platelets. In some embodiments, the pooled platelet preparation may comprise, for example, less than about 16.0×10¹¹, less than about 15.0×10¹¹, less than about 14.0×10¹¹, less than about 13.0×10¹¹, or less than about 12.0×10¹¹ platelets. In some embodiments, the pooled platelet preparation may comprise, for example, about 5.0×10¹¹, about 5.5×10¹¹, about 6.0×10¹¹, about 6.5×10¹¹, about 7.0×10¹¹, about 7.5×10¹¹, about 8.0×10¹¹, about 8.5×10¹¹, about 9.0×10¹¹, about 9.5×10¹¹, about 10.0×10¹¹, about 10.5×10¹¹ about 11.0×10¹¹ about 11.5×10¹¹ about 12.0×10¹¹ about 12.5×10¹¹ about 13.0×10¹¹, about 13.5×10¹¹, about 14.0×10¹¹, about 14.5×10¹¹, or about 15.0×10¹¹ platelets

### Platelet Processing and Processing Sets and Kits

Platelet processing as described in the present disclosure may involve the use of blood product container or blood product bag systems, which are well known in the art. In general, such systems may include more than one plastic container, typically plastic bags, where the bags are integrally connected with plastic tubing. Some of the containers described herein include such plastic bags as are known in the storage and handling of blood products, including platelet products. Blood bags typically can be designed to hold various volumes of fluid, including, but not limited to, volumes ranging from 50 mL to 2 liters, for example having up to a 350 mL capacity, 450 mL capacity, 500 mL capacity, 1 liter capacity, up to a 1.5 liter capacity, or up to a 2 liter capacity. It is understood that when a method refers to a bag, it includes any such plastic bags used in blood product handling. Where such bags are referred to as “pooling bag”, “mixing bag”, “removal bag”, “product bag”, “storage bag”, or “illumination bag”, it is understood that these bags are typical blood product handling bags, or are similar to such bags in nature. Plastic bags suitable for use according to the present disclosure include for example, those comprising PL2410, as well as other suitable plastics known in the art. Plastic bag materials include polyvinyl chloride, polyolefins, ethylene vinyl acetate, ethylene vinyl acetate blended with other plastics, and the like.

As described herein, where tubing is described as connecting e.g. two bags, such as for pooling and/or of a processing set, it is understood that the tubing may be joined at some point therebetween by another component of the connection between the two bags. For example, a removal bag connected to a product bag by a tubing includes wherein the tubing comprises a filter between the two bags, i.e. the tubing is divided by a filter such that fluid flows from one bag to the other through the tubing and filter. In one example, tubing connecting a removal bag and a product bag can include a filter to remove any loose particles from fluid flowing from the removal device to the product bag, i.e. the tubing is divided by, or interrupted by the filter between the bags. Such filters are designed to remove any small particles that may come off of the removal device, while allowing platelets to pass through the filter. The tubing between bags allows for fluid to flow from one bag to another, which can be blocked to prevent the flow until necessary, e.g. as part of the processing the fluid in one bag may be prevented from flowing to the next bag until required for the next step in a process. As such an openable seal, such as a clamp, plug, valve or the like is included in or on the tubing connecting the bags, where the clamp, plug, valve or the like can be selectively opened as required, for example to transfer the fluid from one bag to the next. In certain embodiments, the tubing between bags comprises a breakable seal, such as a breakable valve, whereupon breaking the breakable seal allows for the blood product solution to flow between the bags through the tubing. It is understood that the breakable seal is contained within the connection between containers, such that sterility of the system is maintained. It is also understood that a tubing comprising a filter, or a breakable seal, includes where the tubing may be interrupted by the filter or the seal, for example the tubing runs from one bag and is connected to the filter or seal (an incoming portion of the tubing), and the tubing continues from another portion of the filter or seal to another bag (an outgoing portion of the tubing). In such a configuration, fluid flows from the first bag, through the incoming portion of the tubing, through the filter or seal, and through the outgoing portion of the tubing and into the other bag.

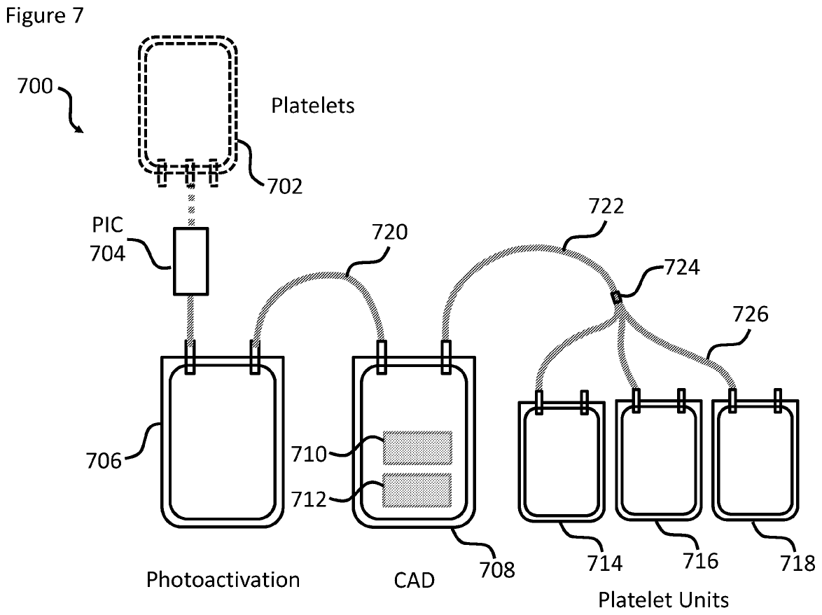

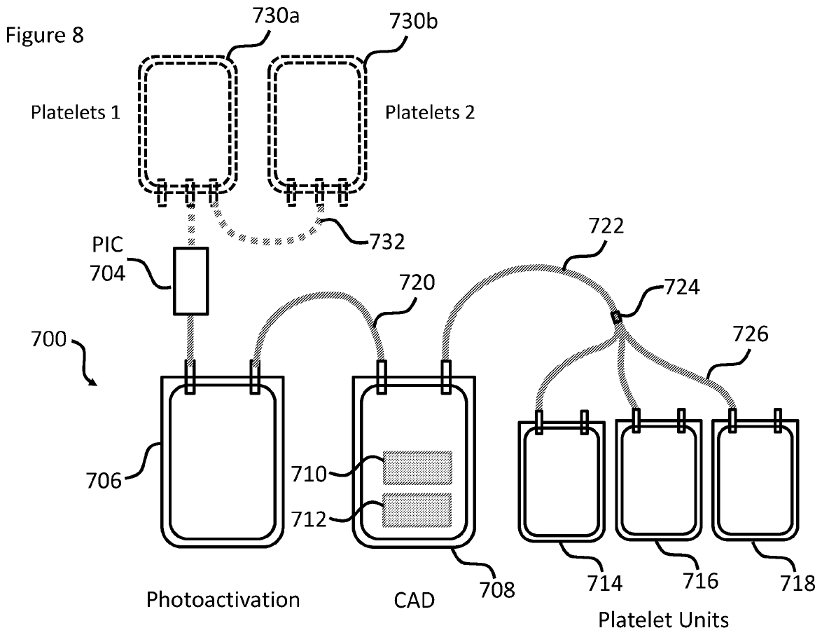

Different bags within a blood product bag system can be used for different steps of a process (see e.g., FIGS. 7-8). For example, a system of bags to be used for the pathogen inactivation of a preparation of platelets can include a container with pathogen inactivating compound contained within, a bag for receiving the unit of platelets (e.g., platelet donation) and a pathogen inactivating compound (e.g. an illumination bag), a bag for the illumination of the unit of platelets when the pathogen inactivation method includes illumination (e.g., an illumination bag, and typically the same bag to receive the unit of platelets and pathogen inactivating compound), a bag for the removal of pathogen inactivating compounds and/or by-products thereof from the treated unit of platelets (e.g., referred to as a removal bag, compound adsorption device, CAD), and one or more bags for containing the final platelet product, i.e. the pathogen inactivated platelet unit (e.g., therapeutic dosage unit) that has the concentration of the inactivating compound and/or by-products thereof reduced to below a desired concentration, which is ready for use or can be stored for later use (e.g., referred to as a product bag, storage bag). Each bag in the system is typically made up of a plastic material. For example, the container for containing a solution of pathogen inactivating compound can be made of a suitable plastic such as PL2411 (Baxter Healthcare), or other plastics such as polyvinyl chloride, polyolefins, ethylene vinyl acetate, ethylene vinyl acetate blended with other plastics, and the like. This container is also overwrapped with a material that is impermeable to light of a wavelength that will activate the photoactive pathogen inactivation compound (for example suitable plastic such as PL2420, Baxter Healthcare). The illumination bag for a photoactivated pathogen inactivating compound requires a clear, durable thermoplastic material that is translucent to light of the selected wavelength. Suitable plastics that are translucent to light in the UVA wavelength range include polyvinyl chloride, polyolefins, ethylene vinyl acetate, ethylene vinyl acetate blended with other plastics, or other blends of thermoplastic polymers. Such suitable plastics include PL2410 (Baxter Healthcare) and PL732 (Baxter Healthcare). Similar materials may be used to make the removal bag and the product bag. The product bags include, for example, those made of PL2410. Suitable bag materials are discussed, for example, in PCT publication number WO 2003078023, and U.S. Pat. No. 7,025,877, the disclosures of which are hereby incorporated by reference as it relates to such bag materials and related materials. In all cases, the materials used in preparing the processing set have to be sterilizable by known methods such as steam and gamma or electron beam radiation used to ensure sterility of the processing set. While these are exemplary materials for making the bags, the methods described herein are applicable to processes using any suitable bag material as would be readily available to one skilled in the art, and can also be used with containers other than bags. The bags used for illumination, removal, and storage are also designed to allow for gases such as oxygen and carbon dioxide to go into and out of the blood bag, so that the platelets therein have adequate oxygen supply and carbon dioxide levels during the processing and storage.

Certain aspects of the present disclosure relate to processing sets. The processing sets of the present disclosure may find use, inter alia, in preparing a plurality of platelet units suitable for infusion, e.g., as described herein. Any of the exemplary components such as bags and tubings described supra may find use in the processing sets of the present disclosure.

In some embodiments, provided herein are processing sets for preparing a plurality of platelet units suitable for infusion. In some embodiments, the processing sets comprise a first container that contains a pathogen inactivation compound (PIC) and is suitable for mixing two or more platelet donations with the PIC; a second container, coupled to the first container, within which the two or more platelet donations in admixture with the PIC can be photochemically inactivated (e.g., as described herein) under sterile conditions (in some embodiments, the volume of the two or more platelet donations is sufficient to yield a plurality of platelet units); a compound adsorption device (CAD) coupled to the second container such that the two or more photochemically inactivated platelet donations can be transferred from the second container to the compound absorption device under sterile conditions (e.g., for reducing the concentration of pathogen inactivating compound); and two or more third containers, each of which is coupled to the compound absorption device such that the two or more photochemically inactivated platelet donations can be transferred from the compound absorption device to the two or more third containers under sterile conditions. In some embodiments, the containers are coupled by a sterile fluid pathway comprising tubing (e.g., sterile tubing). Thus, the processing sets are configured to provide a plurality of pathogen-inactivated platelet units suitable for infusion, e.g., such that the plurality of platelet units comprises a greater number of platelet units than the total number of platelet units that can be prepared from the two or more donations without combining the two or more platelet donations. In some embodiments, the volume of the one or more platelet donations is sufficient to yield a plurality of platelet units, such that the processing sets are configured to process a plurality of pathogen-inactivated platelet units suitable for infusion into a subject. In some such embodiments, a CAD of the present disclosure comprises a container (e.g., CAD container, bag) containing 2 or more CAD wafers (e.g., wafers comprising adsorbent particles). In some embodiments, the processing sets comprise three or more (e.g., three) third containers, each of which is coupled to the compound absorption device such that the two or more photochemically inactivated platelet donations can be transferred from the compound absorption device to the three or more third containers under sterile conditions. In some embodiments, the processing sets are suitable (e.g., configured) for sterile coupling with one or more additional containers that are suitable for containing one or more platelet donations (e.g., wherein containing comprises combining two or more platelet donations), wherein after coupling of the additional container(s) with the processing set, the one or more platelet donations can be transferred from the additional container to the processing set under sterile conditions. For example, in some embodiments, the processing sets comprise a first container suitable for sterile coupling with an additional container. In such a processing set comprising a first container suitable for sterile coupling with an additional container, after coupling the additional container to the first container, the one or more platelet donations can be transferred from the additional container to the first container under sterile conditions. In some embodiments, the processing sets comprise an additional container suitable for containing one or more platelet donations (e.g., wherein containing comprises combining two or more platelet donations), wherein the additional container is coupled to the first container such that the one or more platelet donations can be transferred from the additional container to the first container under sterile conditions. For example, the additional container can be suitable for containing a platelet donation obtained by apheresis. In some embodiments, the additional container is suitable for sterile coupling with a second additional container. In some embodiments, the processing sets comprise multiple additional containers coupled to one another, wherein at least one of the additional containers is coupled to the first container, and wherein platelet donations can be mixed or pooled together in at least one of the additional containers. For example, the additional containers can be suitable for mixing platelets from apheresis donations from multiple donors.

Further non-limiting examples of processing sets are illustrated in FIGS. 7 & 8. Exemplary processing set 700 is shown in FIG. 7. In some embodiments, set 700 includes optional bag 702 containing donor platelets. Processing set 700 includes container 704 (e.g., a first container) that contains a pathogen inactivation compound (PIC, e.g., a psoralen) and is sterile connected to optional container (e.g., bag) 702 (e.g., an additional container) and connected to container (e.g., bag) 706 to allow for exposure of donor platelets to the PIC and sterile transfer of the donor platelets to container 706 for photochemical inactivation (e.g., a second container). Processing set 700 further includes CAD (e.g., CAD container, CAD bag) 708 connected to container 706 via sterile tubing 720, which allows transfer of the donor platelets after photochemical inactivation to the CAD. CAD 708 contains two wafers, 710 and 712, each of which provides for removal or reducing the concentration of pathogen inactivating compound. Advantageously, this allows for pathogen inactivation of a larger number or volume of donor platelets. For example, in some embodiments, 710 and 712 can include adsorbent particles contained in a mesh pouch and/or matrix (e.g., as described infra) that bind and/or otherwise adsorb the pathogen inactivating compound. Processing set 700 further includes sterile tubing 722, three-way lead 724, and three additional sterile tubings (e.g., tubing 726) that connect CAD 708 to platelet unit containers (e.g., bags) 714, 716, and 718 (e.g., three third containers). Advantageously, this configuration allows for the production of an increased number of platelet units (e.g., in bags 714, 716, and 718) from the donor platelets (e.g., if donor platelets 702 are from an apheresis donation).

An alternative configuration for processing set 700 is shown in FIG. 8. Instead of being sterile connected to bag 702 containing donor platelets, container 704 is sterile connected to two containers, 730a and 730b (e.g., first and second additional containers), containing donor platelets (e.g., first donor platelets, second donor platelets). 730a and 730b are sterile connected to each other (e.g., for pooling) via sterile tubing 732. Advantageously, this configuration provides for a pooled platelet preparation that can be subjected to pathogen inactivation and subsequently divided into an increased number of individual platelet units (e.g., 714, 716, and 718).

Certain aspects of the present disclosure also relate to kits. For example, the disclosure provides a kit comprising (a) a processing set (e.g., an aforementioned processing set), and (b) instructions for using the processing set to prepare a plurality of platelet units from two or more platelet donations, wherein each of the platelet units is pathogen-inactivated and suitable for infusion into a subject, wherein the plurality of platelet units comprises a greater number of platelet units than can be prepared from the two or more platelet donations without combining the two or more platelet donations, and wherein the processing set comprises: 1) a first container that contains a pathogen inactivation compound (PIC) and is suitable for combining the two or more platelet donations with the PIC; 2) a second container, coupled to the first container, within which the two or more platelet donations in admixture with the PIC can be photochemically inactivated under sterile conditions, wherein the volume of the two or more platelet donations is sufficient to yield the plurality of platelet units; 3) a compound absorption device (CAD) coupled to the second container such that the two or more photochemically inactivated platelet donations can be transferred from the second container to the compound absorption device under sterile conditions; and 4) two or more third containers, each of which is coupled to the compound absorption device such that the two or more photochemically inactivated platelet donations can be transferred from the compound absorption device to the two or more third containers under sterile conditions to provide the plurality of platelet units. In some embodiments, the kit comprises instructions for using the processing set to prepare a plurality of platelet units from two or more apheresis platelet donations, wherein each of the platelet units is pathogen-inactivated and suitable for infusion into a subject, wherein the plurality of platelet units comprises a greater number of platelet units than can be prepared from the two or more apheresis platelet donations without combining the two or more apheresis platelet donations. In some embodiments, the kit comprises three or more (e.g., three) third containers, each of which is coupled to the compound absorption device such that the two or more photochemically inactivated platelet donations can be transferred from the compound absorption device to the three or more (e.g., three) third containers under sterile conditions to provide the plurality of pathogen-inactivated platelet units.

In some embodiments, the first container is suitable for sterile coupling with an additional container suitable for containing one or more of the two or more platelet donations (e.g., platelet donation obtained by apheresis) such that the one or more platelet donations of the additional container can be transferred from the additional container to the first container under sterile conditions. In some embodiments, the additional container is suitable for sterile coupling with a second additional container. In some embodiments, the second additional container contains one or more of the two or more platelet donations, and wherein the additional container is suitable for sterile coupling with the second additional container such that the one or more platelet donations of the second additional container can be transferred from the second additional container to the additional container under sterile conditions. In some embodiments, the platelet units in the plurality of platelet units each comprise a therapeutic dose of platelets.

In some embodiments, the instructions for using the processing set to prepare a plurality of platelet units from two or more platelet donations provide a minimum and/or maximum number of platelets (e.g., combined platelet number) from the two or more platelet donations suitable for combining with the PIC. In some embodiments, the instructions for using the processing set to prepare a plurality of platelet units from two or more platelet donations provide a minimum and/or maximum volume (e.g., combined volume) for the two or more platelet donations suitable for combining with the PIC. In some embodiments, the minimum number of platelets is about 4.5×10¹¹, about 5.0×10¹¹, about 5.5×10¹¹, about 6.0×10¹¹, about 6.5×10¹¹, about 7.0×10¹¹ platelets. In some embodiments, the maximum number of platelets is about 15.0×10¹¹, about 14.0×10¹¹, about 13.0×10¹¹, about 12.0×10¹¹, about 11.0×10¹¹ platelets. In some embodiments, the minimum number of platelets is about 4.0×10¹¹ platelets and the maximum number of platelets is about 13.0×10¹¹ platelets. In some embodiments, the minimum number of platelets is about 5.0×10¹¹ platelets and the maximum number of platelets is about 12.0×10¹¹ platelets. In some embodiments, the minimum number of platelets is about 6.0×10¹¹ platelets and the maximum number of platelets is about 12.0×10¹¹ platelets. In some embodiments, the minimum volume of platelets is about 360 mL, about 380 mL, about 400 mL, about 420 mL, about 440 mL, about 460 mL, about 480 mL, or about 500 mL. In some embodiments, the maximum volume of platelets is about 750 mL, about 700 mL, about 675 mL, about 650 mL, about 625 mL, or about 600 mL. In some embodiments, the minimum volume of platelets is about 400 mL and the maximum volume of platelets is about 700 mL. In some embodiments, the minimum volume of platelets is about 420 mL and the maximum volume of platelets is about 650 mL. In some embodiments, the minimum volume of platelets is about 440 mL and the maximum volume of platelets is about 625 mL.

Pathogen Inactivation

Blood products, including platelet-containing blood products, may contain pathogens, or may be contaminated with pathogens during processing. As such, it is desirable to subject such blood products to a pathogen inactivation process in order to reduce the risk of transfusion-transmitted diseases. Various processes and methods have been assessed to mitigate the risk of transfusion-associated disease transmission in platelet-containing blood products. Aside from screening and detection of pathogens and subsequent elimination of contaminated blood products, processes that incorporate treatments to inactivate pathogens (i.e., pathogen inactivation) that may be present are available. Ideally, such a process results in the inactivation of a broad range of pathogens such as viruses, bacteria and parasites that may be present in the blood product. In certain preferred embodiments, the methods of pathogen inactivation require addition of an amount of pathogen inactivating compound to a preparation of platelets (e.g., treating the platelet preparation). For example, pathogen inactivation may involve the addition of a low molecular weight compound that inactivates various pathogens, where a preferred method involves the addition of a photosensitizer that, when activated by illumination using light of suitable wavelengths, will inactivate a variety of pathogens that may be present. Two preferred methods that are commercially available include the addition of amotosalen or riboflavin to the platelets, with subsequent illumination with UV light. Other methods include illumination with UV light without addition of a photosensitizer, as well as illumination with other photoactive compounds, including psoralen derivatives other than amotosalen, isoalloxazines other than riboflavin, alloxazines, dyes such as phthalocyanines, phenothiazine dyes (e.g. methylene blue, azure B, azure C, thionine, toluidine blue), porphyrin derivatives (e.g. dihematoporphyrin ether, hematoporphyrin derivatives, benzoporphyrin derivatives, alkyl-substituted sapphyrin), and merocyanine 540 (Prodouz et al., Blood Cells 1992, 18(1):101-14; Sofer, Gail, BioPharm, August 2002). Other pathogen inactivation systems include, for example, those described in PCT publication numbers WO 2012071135; WO 2012018484; WO 2003090794; WO 2003049784; WO 1998018908; WO 1998030327; WO 1996008965; WO 1996039815; WO 1996039820; WO 1996040857; WO 1993000005; US patent application number US 20050202395; and U.S. Pat. Nos. 8,296,071 and 6,548,242, the disclosures of which are hereby incorporated by reference as they relate to pathogen inactivation in blood products. In some embodiments, the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540. In some embodiments, the pathogen inactivating compound is a psoralen. In some embodiments, the pathogen inactivating compound is amotosalen. Where addition of a compound to the platelets is used for pathogen inactivation, whether the method requires illumination or not, in some instances it is desirable to remove any residual pathogen inactivation compound or by-product thereof.

Methods for pathogen inactivation and removal of pathogen inactivating compound as described herein are applicable to any platelet preparations, whether the platelet preparations comprise individual platelet donations (e.g., apheresis collected platelets) or pooled platelet preparations. These processes typically provide a platelet preparation that is either in about 85% to 100% plasma or has some amount of platelet additive solution added, typically in the range of 50 to 95% platelet additive solution, with the rest of the volume effectively being plasma, i.e. plasma in the range of about 5 to 50%. It is understood that a solution of pathogen inactivating compound can be added during the processing to inactivate pathogens, since pathogen inactivating compound is not typically combined in solid form, but is dissolved in a solution (for example, amotosalen is the HCl salt dissolved in a saline solution). As such, in some instances, when a platelet preparation is designated as about 100% plasma, it is understood that this means no additional platelet additive solution is included in the platelet unit. If such a preparation of platelets in about 100% plasma is treated for pathogen inactivation, some volume of the solution of pathogen inactivating compound will be included in the final product, as well as some volume of anticoagulant used in collecting the blood for isolation of platelets. While the plasma has been diluted partially with whatever amount of anticoagulant and solution that is used to contain the pathogen inactivating compound, the resulting platelet preparation including pathogen inactivation compound may be referred to as comprising about 100% plasma, or may be referred to as about 85 to 100% plasma (typically less than about 5 to 15% of the volume will comprise the solution used to deliver the pathogen inactivating compound). Platelet preparations can also be prepared with some amount of platelet additive solution, which may, for example, be added after concentrating the platelets, removing a portion of the plasma from the supernatant, and adding the desired amount of platelet additive solution to the platelet preparation. The platelet additive is added to provide the desired percentage of platelet additive solution. Such a preparation of platelets is typically adjusted so the plasma content is about 5 to 50%, with the remainder of the solution being platelet additive solution, i.e. 50 to 95% platelet additive solution. When amounts of plasma and platelet additive solutions are described, it is understood that as with platelet preparations described as being in about 100% plasma, some volume of solution containing a pathogen inactivating compound may be included in the unit of platelets containing a pathogen inactivating compound. While the solution has been diluted partially with whatever amount of solution is used to contain the pathogen inactivating compound, it is understood that, for example, a platelet preparation designated as comprising 35% plasma and 65% platelet additive solution may refer to relative amounts of plasma and platelet additive solution prior to the addition of a solution containing pathogen inactivating compound.

Some pathogen inactivation methods may require the use of a removal device, i.e. a device for reducing the concentration of pathogen inactivating compound, such as a small organic compound, e.g. platelet inactivating compound, and by-products thereof in a preparation of platelets, while substantially maintaining a desired biological activity of the platelets. In some embodiments, the removal device is referred to as a compound adsorption device (CAD), and may comprise a container (e.g., CAD container, CAD bag) containing one or more materials, such as for example, adsorbent particles, and which is suitable for also containing a preparation of platelets from which the concentration of pathogen inactivating compound and by-products thereof are to be reduced. Such a removal device is generally intended to be used in a batch mode, i.e. the device is placed in contact with the platelets, and continued contact with the removal device, e.g. with shaking to allow essentially the entirety of the solution of platelets to come into contact with the removal device over time of contact, results in reducing the levels of pathogen inactivating compound. Such batch devices entail the use of an adsorbent particle that binds the pathogen inactivation compound, and can be used by either adding adsorbent particles directly to the platelet container (e.g., bag) following illumination or transferring the platelets to a bag containing the adsorbent particles following illumination and the platelets are then agitated for a specified period of time with the platelet preparations contacting the removal device. While free adsorbent particles may be used as a removal device, such particles may be contained within a mesh pouch, such as a polyester or nylon mesh pouch, which allows for contact of the platelet solution with the adsorbent particles while containing the particles within the pouch. Alternatively, the adsorbent particles may be immobilized within a matrix, where the immobilized matrix can reside directly in the blood bag used for batch removal, or may be similarly contained within a mesh pouch. In some instances, the removal device comprises porous adsorbent particles in an amount sufficient to reduce the pathogen inactivating compound to below a desired concentration, wherein the adsorbent particles have an affinity for the pathogen inactivating compound, where it is understood such adsorbent particle can be selected to best adsorb the compound or compounds to be removed, with minimal effect on components that should not be removed or damaged by contact with the adsorbent particle. A variety of adsorbent particles are known, including generally particles made from any natural or synthetic material capable of interacting with compounds to be removed, including particulates made of natural materials such as activated carbon, silica, diatomaceous earth, and cellulose, and synthetic materials such as hydrophobic resins, hydrophilic resins or ion exchange resins. Such synthetic resins include, for example, carbonaceous materials, polystyrene, polyacrylic, polyacrylic ester, cation exchange resin, and polystyrene-divinylbenzene. Detailed description of such removal devices suitable for use in the methods as described herein can be found in PCT publication numbers WO 1996040857, WO 1998030327, WO 1999034914, and WO 2003078023, the disclosures of which are hereby incorporated by reference with respect to the discussion of such removal devices and the adsorbent particles and other materials used to prepare such devices. Exemplary adsorbent particles include, but are not limited to, Amberlite (Rohm and Haas) XAD-2, XAD-4, XAD-7, XAD-16, XAD-18, XAD-1180, XAD-1600, XAD-2000, XAD-2010; Amberchrom (Toso Haas) CG-71m, CG-71c, CG-161m, CG161c; Diaion Sepabeads (Mitsubishi Chemicals) HP20, SP206, SP207, SP850, HP2MG, HP20SS, SP20MS; Dowex (Dow Chemical) XUS-40285, XUS-40323, XUS-43493 (also referred to as Optipore V493 (dry form) or Optipore L493 (hydrated form)), Optipore V503, Optipore SD-2; Hypersol Macronet (Purolite) MN-100, MN-102, MN-150, MN-152, MN-170, MN-200, MN-202, MN-250, MN-252, MN-270, MN-300, MN-400, MN-500, MN-502, Purosorb (Purolite) PAD 350, PAD 400, PAD 428, PAD 500, PAD 550, PAD 600, PAD 700, PAD 900, and PAD 950. The material used to form the immobilized matrix comprises a low melting polymer, such as nylon, polyester, polyethylene, polyamide, polyolefin, polyvinyl alcohol, ethylene vinyl acetate, or polysulfone. In one example, the adsorbent particles immobilized in a matrix are in the form of a sintered medium. While it is understood that the methods and devices described herein encompass removal devices as are known in the art, such methods and devices may be exemplified using the removal device of an amotosalen inactivated platelet product as is commercially available. Such a removal device comprises Hypersol Macronet MN-200 adsorbent contained within a sintered matrix, where the sintered matrix comprises PL2410 plastic as a binder. In one instance, the removal device comprises Hypersol Macronet MN-200 adsorbent in a sintered matrix comprising PL2410, wherein the Hypersol Macronet MN-200 is in an amount of about 5-50 grams, about 5-10 grams, about 10-15 grams, about 15-20 grams, about, 20-25 grams, about 25-30 grams, about 30-35 grams, about 35-40 grams, about 40-45 grams or about 45-50 grams dry weight equivalent.

As various resins may require different processing when used to make the removal devices useful in the methods and devices as described herein, comparison of amounts of adsorbent resins described herein, unless otherwise indicated, are comparison of the dry weight of the resin. For example, the resins are dried to <5% water prior to processing, and the equivalent of the dry weight of adsorbent is used in comparing amounts of resin in use. For example, Hypersol Macronet MN-200 is processed to stabilize the adsorbent, or what is typically referred to as wetting the adsorbent, so as to be directly usable upon contact with a platelet unit. Such a wetted sample may include, for example, about 50% glycerol or other suitable wetting agent. In some embodiments, the adsorbent resin is a polystyrene-divinylbenzene resin. In some embodiments, the polystyrene-divinylbenzene resin is Hypersol Macronet MN-200. In some embodiments, the adsorbent is contained within a sintered matrix, wherein the sintered matrix comprises PL2410 binder. In some embodiments, Hypersol Macronet MN-200 adsorbent is contained within a sintered matrix to provide a removal device.

### Platelet Units

The present disclosure also provides platelet units suitable for infusion (e.g., infusion into a human subject), such as for example a platelet unit selected from a plurality of platelet units prepared by any of the methods of the present disclosure. The platelet units (e.g., each platelet unit) in a plurality of platelet units comprise a therapeutic dose (e.g., therapeutic dosage unit) of platelets suitable for infusion into a human subject (e.g., a subject in need of a platelet infusion). In some embodiments, the therapeutic dose comprises a minimum number (e.g., at least a minimum number) of platelets as defined by criteria (e.g., acceptance criteria) of a governmental agency, regulatory agency, institution and/or accrediting organization (e.g., governmental agency, regulatory agency, institution and/or accrediting organization for donated blood products (e.g., donated platelets)). In some embodiments, the platelet units are prepared in the country of the governmental agency, regulatory agency, institution and/or accrediting organization defining the criteria of a therapeutic dose of platelets. In some embodiments, at least about 75%, at least about 80%, at least about 85%, at least about 90%, at least about 95%, at least about 98%, at least about 99% or more of the platelet units in the plurality of platelet units comprise the minimum number of platelets of a therapeutic dose. In some embodiments, each of the platelet units in the plurality of platelet units comprises the minimum number of platelets of a therapeutic dose. In some embodiments, the minimum number of platelets in a therapeutic dose is at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ platelets. In some embodiments, the platelet units in a plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ or more platelets. In some embodiments, each of the platelet units in a plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ or more platelets. In some embodiments, at least about 75% of the platelet units in a plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ or more platelets. In some embodiments, at least about 80% of the platelet units in the plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ or more platelets. In some embodiments, at least about 85% of the platelet units in the plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ or more platelets. In some embodiments, at least about 90% of the platelet units in the plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ or more platelets. In some embodiments, at least about 95% of the platelet units in the plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ or more platelets. In some embodiments, at least about 98% of the platelet units in the plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ or more platelets. In some embodiments, at least about 99% of the platelet units in the plurality of platelet units comprise at least about 2.0×10¹¹ platelets, at least about 2.2×10¹¹ platelets, at least about 2.4×10¹¹ platelets, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets or at least about 3.0×10¹¹ or more platelets.

In another aspect, the present disclosure provides a therapeutic dosage unit of platelets suitable for infusion into a subject, wherein the therapeutic dosage unit comprises pooled apheresis-derived platelets from two or more donors, and wherein the pooled apheresis-derived platelets have been treated with a pathogen inactivating compound. In some embodiments, the apheresis-derived platelets have been treated with the pathogen inactivating compound prior to pooling. In some embodiments, the apheresis-derived platelets have been treated with the pathogen inactivating compound after pooling. In some embodiments, the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540. In some embodiments, the pathogen inactivating compound is a psoralen. In some embodiments, the pathogen inactivating compound is amotosalen. In some embodiments, the apheresis-derived platelets are from donors of the same ABO blood type. In some embodiments, the apheresis-derived platelets are from donors of the same ABO and Rh type. In some embodiments, the therapeutic dosage unit of platelets comprises at least about 2.0×10¹¹ platelets, at least about 2.2×10¹¹ platelets, at least about 2.4×10¹¹, at least about 2.5×10¹¹ platelets, at least about 2.6×10¹¹ platelets, at least about 2.7×10¹¹ platelets, at least about 2.8×10¹¹ platelets, at least about 2.9×10¹¹ platelets, at least about 3.0×10¹¹ or more platelets. In some embodiments, the therapeutic dosage unit of platelets comprises at least 2.4×10¹¹ platelets. In some embodiments, the therapeutic dosage unit of platelets comprises at least 2.6×10¹¹ platelets. In some embodiments, the therapeutic dosage unit of platelets comprises at least 3.0×10¹¹ platelets. In some embodiments, the therapeutic dosage unit of platelets comprises a minimum number (e.g., at least a minimum number) of platelets as defined by criteria (e.g., acceptance criteria) of a governmental agency, regulatory agency, institution and/or accrediting organization (e.g., governmental agency, regulatory agency, institution and/or accrediting organization for donated blood products (e.g., donated platelets)). In some embodiments, the therapeutic dosage unit of platelets is prepared in the country of the governmental agency, regulatory agency, institution and/or accrediting organization defining the criteria of a therapeutic dosage unit of platelets.

The present disclosure also provides a method of infusing platelets into a subject (e.g., human subject) in need thereof, comprising infusing into the subject an aforementioned platelet unit or an aforementioned therapeutic dosage unit of platelets.

### Systems

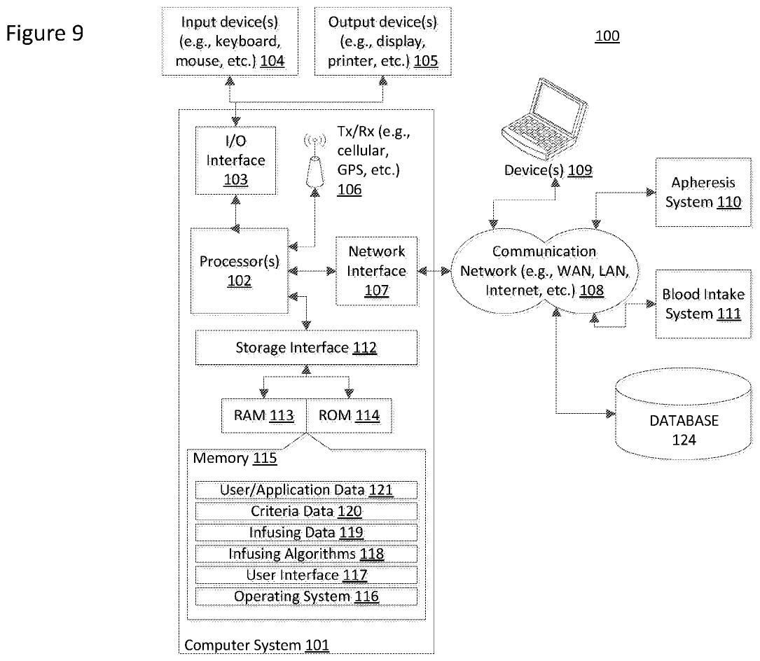

In other aspects, the present disclosure provides computer-implemented methods and systems for optimization of platelet units for infusion. FIG. 9 illustrates an exemplary system 100 for blood infusing optimization (e.g., optimization of platelet units for infusion), consistent with some embodiments of the present disclosure. In some embodiments, optimization comprises optimizing the number of platelet units that can be prepared, such as for example, from a platelet donation and a plurality of platelet preparations (e.g., inventory of platelet preparations), relative to the process for preparing platelet units before optimizing. In some embodiments, optimization comprises reducing excess platelet platelets per platelet unit (e.g., relative to the process for preparing platelet units before optimizing). In some embodiments, optimization comprises reducing or eliminating platelet waste (e.g., relative to the process for preparing platelet units before optimizing). In some embodiments, optimization comprises improving or increasing the number of platelet units prepared, such as for example, from a platelet donation and a plurality of platelet preparations (e.g., inventory of platelet preparations), relative to the process for preparing platelet units before optimizing. In some embodiments, optimization comprises increasing the efficiency of preparing platelet units (e.g., relative to the process for preparing platelet units before optimizing). In some embodiments, optimization comprises obtaining a target value (e.g., number) of platelets per platelet unit.

Referring to FIG. 9, system 100 may include a computer system 101, input devices 104, output devices 105, devices 109, apheresis system 110, and blood intake system 111. It is appreciated that one or more components of system 100 can be separate systems or can be integrated systems. In some embodiments, computer system 101 may comprise one or more central processing units (“CPU” or “processor(s)”) 102. Processor(s) 102 may comprise at least one data processor for executing program components for executing user- or system-generated requests. A user may include a person, a person using a device such as those included in this disclosure, or such a device itself. The processor may include specialized processing units such as integrated system (bus) controllers, memory management control units, floating point units, graphics processing units, digital signal processing units, etc. The processor may include a microprocessor, such as AMD Athlon, Duron or Opteron, ARM's application, embedded or secure processors, IBM PowerPC, Intel's Core, Itanium, Xeon, Celeron or other line of processors, etc. The processor 102 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor(s) 102 may be disposed in communication with one or more input/output (I/O) devices via I/O interface 103. I/O interface 103 may employ communication protocols/methods such as, without limitation, audio, analog, digital, monoaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.11 a/b/g/n/x, Bluetooth, cellular (e.g., code-division multiple access (CDMA), high-speed packet access (HSPA+), global system for mobile communications (GSM), long-term evolution (LTE), WiMax, or the like), etc.

Using I/O interface 103, computer system 101 may communicate with one or more I/O devices. For example, input device 104 may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, visors, electrical pointing devices, etc. Output device 105 may be a printer, fax machine, video display (e.g., cathode ray tube (CRT), liquid crystal display (LCD), light-emitting diode (LED), plasma, or the like), audio speaker, etc. In some embodiments, a transceiver 106 may be disposed in connection with the processor(s) 102. The transceiver may facilitate various types of wireless transmission or reception. For example, the transceiver may include an antenna operatively connected to a transceiver chip (e.g., Texas Instruments WiLink WL1283, Broadcom BCM4750IUB8, Infineon Technologies X-Gold 618-PMB9800, or the like), providing IEEE 802.11a/b/g/n, Bluetooth, FM, global positioning system (GPS), 2G/3G HSDPA/HSUPA communications, etc.

In some embodiments, processor(s) 102 may be disposed in communication with a communication network 108 via a network interface 107. Network interface 107 may communicate with communication network 108. Network interface 107 may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, etc. Communication network 108 may include, without limitation, a direct interconnection, local area network (LAN), wide area network (WAN), wireless network (e.g., using Wireless Application Protocol), the Internet, etc. Using network interface 107 and communication network 108, computer system 101 may communicate with devices 109. These devices may include, without limitation, personal computer(s), server(s), fax machines, printers, scanners, various mobile devices such as cellular telephones, smartphones (e.g., Apple iPhone, Blackberry, Android-based phones, etc.), tablet computers, eBook readers (Amazon Kindle, Nook, etc.), laptop computers, notebooks, gaming consoles (Microsoft Xbox, Nintendo DS, Sony PlayStation, etc.), or the like. In some embodiments, computer system 101 may itself embody one or more of these devices.

In some embodiments, using network interface 107 and communication network 108, computer system 101 may communicate with apheresis system 110, blood intake system 111, or any other medical systems. Computer system 101 may communicate with these systems to obtain data for blood infusing optimization (e.g., optimization of platelet units for infusion). Computer system 101 may also be integrated with these imaging systems.

In some embodiments, processor 102 may be disposed in communication with one or more memory devices (e.g., RAM 113, ROM 114, etc.) via a storage interface 112. The storage interface may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory devices may further include a drum, magnetic disc drive, magneto-optical drive, optical drive, redundant array of independent discs (RAID), solid-state memory devices, flash devices, solid-state drives, etc.

The memory devices may store a collection of program or database components, including, without limitation, an operating system 116, user interface application 117, infusing algorithms 118, infusing data 119, criteria data 120, user/application data 121 (e.g., any data variables or data records discussed in this disclosure), etc. Operating system 116 may facilitate resource management and operation of computer system 101. Examples of operating systems include, without limitation, Apple Macintosh OS X, Unix, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like. User interface 117 may facilitate display, execution, interaction, manipulation, or operation of program components through textual or graphical facilities. For example, user interfaces may provide computer interaction interface elements on a display system operatively connected to computer system 101, such as cursors, icons, check boxes, menus, scrollers, windows, widgets, etc. Graphical user interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, Javascript, AJAX, HTML, Adobe Flash, etc.), or the like.

In some embodiments, computer system 101 may implement infusing algorithms 118. Computer system 101 may also store the infusing data 119 and criteria data 120. For example, memory 115 and/or a frame buffer (not shown) may be used for storing infusing data 119.

In some embodiments, computer system 101 may store user/application data 121, such as data, variables, and parameters as described in this disclosure, and database 124. Database 124 may include data associated with supplies (e.g., platelet preparations) stored in a platelets inventory (e.g., platelet preparation inventory). Database 124 may be internal or external to computer system 101. Such databases may be implemented as fault-tolerant, relational, scalable, secure databases such as Oracle or Sybase. Alternatively, such databases may be implemented using standardized data structures, such as an array, hash, linked list, struct, structured text file (e.g., XML), table, or as object-oriented databases (e.g., using ObjectStore, Poet, Zope, etc.). Such databases may be consolidated or distributed, sometimes among the various computer systems discussed above in this disclosure. It is to be understood that the structure and operation of any computer or database component may be combined, consolidated, or distributed in any working combination.

Disclosed embodiments describe systems and methods for blood infusing optimization (e.g., optimization of platelet units for infusion). The illustrated components and steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items.

It should be noted that, despite references to particular computing paradigms and software tools herein, the computer program instructions with which embodiments of the present subject matter may be implemented may correspond to any of a wide variety of programming languages, software tools and data formats, and be stored in any type of volatile or nonvolatile, non-transitory computer-readable storage medium or memory device, and may be executed according to a variety of computing models including, for example, a client/server model, a peer-to-peer model, on a stand-alone computing device, or according to a distributed computing model in which various of the functionalities may be effected or employed at different locations. In addition, references to particular algorithms herein are merely by way of examples. Suitable alternatives or those later developed known to those of skill in the art may be employed without departing from the scope of the subject matter in the present disclosure.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects. Rather, the scope of this disclosure should be determined with reference to the appended claims.

The invention is illustrated further by the following examples, which are not to be construed as limiting the invention in scope or spirit to the specific procedures described in them.

### EXAMPLES

### Example 1: Apheresis Collection of Platelets

Donor(s) suitable for apheresis donation of platelet are identified and platelets are collected using an approved device with apheresis collection procedures known in the art. More specifically, in one example platelet donations were collected by apheresis using an AMICUS™ system (Fenwal, Inc., Lake Zurich, Ill.). Gender, weight, height, hematocrit and platelet pre-count were determined for available donors and the donors were targeted for a single, double or triple apheresis collections to prepare 1, 2 or 3 platelet units, each comprising at least the number of platelets per unit according to applicable FDA regulatory standards. The apheresis collections factored in potential losses (e.g., processing losses) and split ranges for the blood center were >3.1×10¹¹ platelets for a single, >6.2×10¹¹ platelets for a double, and >9.6×10¹¹ platelets for a triple. The corresponding ranges for such splits also may be represented, for example, as >3.1×10¹¹ to <6.2×10¹¹ platelets for a single, >6.2×10¹¹ to <9.6×10¹¹ platelets for a double, and >9.6×10¹¹ to <12.8×10¹¹ platelets for a triple.

Table 1 illustrates donor characterization and targeted platelet collections (target platelet yield) in order to prepare the indicated number of platelet products for each of twelve donors. Preparation of apheresis platelet units by standard methods as the targeted singles, doubles or triples resulted in 23 platelet units from the above twelve donors.

TABLE 1
Apheresis platelet collections

| | | Weight | Height | Pre- | Target | |
|---|---|---|---|---|---|---|
| Donor | Gender | (lbs) | (in.) | count | Yield | Products |
| 1 | M | 185 | 75″ | 375 | 10.96 | 3 |
| 2 | M | 167 | 71″ | 257 | 7.20 | 2 |
| 3 | M | 170 | 70″ | 246 | 6.38 | 2 |
| 4 | F | 202 | 66″ | 433 | 10.50 | 3 |
| 5 | F | 165 | 63″ | 309 | 7.80 | 2 |
| 6 | M | 210 | 70″ | 309 | 8.75 | 2 |
| 7 | M | 175 | 72″ | 205 | 5.21 | 1 |
| 8 | F | 165 | 64″ | 441 | 7.00 | 2 |
| 9 | F | 135 | 67″ | 220 | 4.50 | 1 |
| 10 | M | 212 | 73″ | 234 | 8.00 | 2 |
| 11 | M | 140 | 67″ | 190 | 4.86 | 1 |
| 12 | M | 155 | 66″ | 362 | 9.15 | 2 |

Among these twelve donors, platelet collections for donors #6, 7, 11 and 12 may be categorized as extra-large collections as provided in one embodiment of the present disclosure, in that each comprises excess platelet numbers, in this case greater than 50% platelet above the platelet split number, but less than the platelet number to achieve the next higher split level to prepare an additional platelet unit product (e.g., based on platelet units of at least 3×10¹¹). For example, donor #6 with 8.75×10¹¹ platelets, exceeded the >6.2×10¹¹ yield for a double collection but is less than the 9.6×10¹¹ platelets for a triple collection (e.g., an extra-large double). Similarly, donor #11 with 4.86×10¹¹ platelets, exceeded the >3.1×10¹¹ yield for a single collection but is less than the >6.2×10¹¹ yield for a double collection (e.g., an extra-large single).

### Example 2: Preparing Increased Number of Platelet Units

As described in one embodiment of the present disclosure, the methods provided herein set forth a method of preparing a plurality of platelet units comprising a greater number of platelet units than the total number of platelet units that can be prepared from the same apheresis platelet donations without using the disclosed methods. More specifically, in one example, an increased number of platelet units are prepared from the twelve donors of Example 1 and Table 1. Following collection, the following pairs of platelet donations are combined:

Donors #7 and 11, each comprising greater than the number of platelets required to prepare one platelet unit, but less than the number of platelets required to prepare two platelet units.
Donors #6 and 12, each comprising greater than the number of platelets required to prepare two platelet units, but less than the number of platelets required to prepare three platelet units.

Following the combining of the platelet donations pairs, the pooled platelet preparations are separated into three platelet units (in the case of #7 with #11), and five platelet units (in the case of #6 and #12). Together with the units (e.g., non-pooled units) from the remaining eight donors from Table 1, these methods provide for an increased number of apheresis platelet units, with 25 total platelet units prepared, as compared to 23 platelet units that would result from convention apheresis collection and preparation methods (e.g., based on platelet units of at least 3×10¹¹). Thus, an increased platelet unit production of almost 9% based on the present disclosure, without additional donors or platelet donations.

Similarly, alternative combinations of the platelet donations may be combined to achieve and increased number of total platelet units prepared. For example, following collection, the following pairs of platelet donations are combined:

Donors #6 and 7, with #6 comprising greater than the number of platelets required to prepare two platelet units, but less than the number of platelets required to prepare three platelet units, and #7 comprising greater than the number of platelets required to prepare one platelet unit, but less than the number of platelets required to prepare two platelet units.
Donors #11 and 12, with #12 comprising greater than the number of platelets required to prepare two platelet units, but less than the number of platelets required to prepare three platelet units, and #11 comprising greater than the number of platelets required to prepare one platelet unit, but less than the number of platelets required to prepare two platelet units

Following the combining of the platelet donations pairs, the pooled platelet preparations are separated into four platelet units (in the case of #6 with #7), and four platelet units (in the case of #11 and #12). Together with the units (e.g., non-pooled units) from the remaining eight donors from Table 1, these methods again provide for an increased number of apheresis platelet units, with 25 total platelet units prepared, as compared to 23 platelet units that would result from convention apheresis collection and preparation methods (e.g., based on platelet units of at least 3×10¹¹). Analysis of a significantly larger apheresis platelet donation data set provides an opportunity to further highlight the improved preparation of a plurality of platelet units, with a significantly greater number of platelet units that may be prepared according to methods of the present disclosure, as compared to currently accepted apheresis collection practices for preparing platelet units.

### Example 3: Pathogen Inactivation

The methods described in Example 2 may further include a pathogen inactivation treatment step. More specifically, in one embodiment, the apheresis collected platelets are individually subjected to a pathogen inactivation process with a pathogen inactivating compound, using the commercially available INTERCEPT® Blood System for platelets (Cerus Corporation, Concord, Calif.), according to manufacturer instructions. Following the pathogen inactivation treatment of the platelet donations from donors #6, 7, 11 and 12, the platelet donations are combined in pairs as set forth above, and the pooled platelet preparations are separated into three platelet units. Each of the other eight platelet donations are similarly subjected to the pathogen inactivation process, providing a plurality of pathogen inactivated platelet units. Alternatively, the pooled platelet preparation may be subjected to a pathogen inactivation process with a pathogen inactivating compound, using the commercially available INTERCEPT® Blood System for platelets, together with a processing set, such as depicted in FIG. 7 or FIG. 8.

In another example, pools of two apheresis platelet donations (n=6) were prepared to contain between approximately 10.2×10¹¹ to 11.8×10¹¹ platelets in approximately 623 to 648 mL of 35% plasma/65% PAS-3 platelet additive solution. The pooled apheresis platelets were subjected to photochemical pathogen-inactivation with amotosalen and UVA, using a processing set as provided in the present disclosure (see e.g., FIGS. 7 and 8) to generate three platelet units of at least 3×10¹¹ platelets from each of the two input apheresis platelet donations. The units were evaluated on Day 7 post-donation for product dose and volume retention, pH, and in vitro platelet quality (e.g., function) parameters correlating with platelet survival and recovery in vivo (e.g., pH, normalized lactate, ATP, morphology score, ESC and HSR), and found to meet EDQM requirements and to be within the ranges for platelet units from single donors and pathogen-inactivated with the commercially available INTERCEPT® Blood System for platelets.

### Example 4: Preparing Further Increased Number of Platelet Units

The platelet preparation methods disclosed herein may be further improved by targeting increased apheresis platelet collections from available donors. For example, in one embodiment, a maximum platelet yield is determined for the above twelve donors based on the previously described donor characteristics. In addition to the prior target yield and targeted products reproduced below from Table 1, the maximum yield platelet number is also illustrated in the following Table 2, as well as the additional targeted maximum product numbers that may be achieved by collecting at the maximum amount of platelets.

TABLE 2

| | | | | Pre- | Target | Targeted | | Max |
|---|---|---|---|---|---|---|---|---|
| Donor | Gender | Weight | Height | count | Yield | Products | Max Yield | Products |
| 1 | M | 185 | 75″ | 375 | 10.96 | 3 | 11.74 | 3 |
| 2 | M | 167 | 71″ | 257 | 7.20 | 2 | 9.39 | 2 |
| 3 | M | 170 | 70″ | 246 | 6.38 | 2 | 8.16 | 2 |
| 4 | F | 202 | 66″ | 433 | 10.50 | 3 | 11.52 | 3 |
| 5 | F | 165 | 63″ | 309 | 7.80 | 2 | 9.09 | 2 |
| 6 | M | 210 | 70″ | 309 | 8.75 | 2 | 11.56 | 3 |
| 7 | M | 175 | 72″ | 205 | 5.21 | 1 | 7.21 | 2 |
| 8 | F | 165 | 64″ | 441 | 7.00 | 2 | 9.90 | 3 |
| 9 | F | 135 | 67″ | 220 | 4.50 | 1 | 6.05 | 1 |
| 10 | M | 212 | 73″ | 234 | 8.00 | 2 | 9.49 | 2 |
| 11 | M | 140 | 67″ | 190 | 4.86 | 1 | 5.81 | 1 |
| 12 | M | 155 | 66″ | 362 | 9.15 | 2 | 9.83 | 3 |

As shown in Table 2, four additional platelet units may be prepared by increasing the apheresis collections to the maximum amount shown, increasing the total to 27 platelet units, as compared to 23 units with previous collection targets. Additionally, as provided in the present disclosure a plurality of even more platelet units may be prepared from the same twelve donors, based on the maximum collections and the methods previously described in Example 2. More specifically, following collection for maximum yield, the following pairs of platelet donations are combined:

Donors #9 and 11, each comprising greater than the number of platelets required to prepare one platelet unit, but less than the number of platelets required to prepare two platelet units.
Donors #2 and 5, each comprising greater than the number of platelets required to prepare two platelet units, but less than the number of platelets required to prepare three platelet units.

Following the combining of the platelet donations pairs, the pooled platelet preparations are separated into three platelet units (in the case of #9 with #11), and five platelet units (in the case of #2 and #5). Together with units (e.g., unpooled units) from the remaining eight donors, these methods provide for an increased number of apheresis platelet units, with 29 total platelet units prepared, as compared to 23 platelet units from conventional apheresis collection and preparation methods or 27 platelet units from convention apheresis collection at the maximum yield (e.g., based on platelet units of at least 3×10¹¹). Analysis of a significantly larger apheresis platelet donation data set provides an opportunity to further highlight the improved preparation of a plurality of platelet units, with a significantly greater number of platelet units that may be prepared according to methods of the present disclosure, as compared to currently accepted apheresis collection practices for preparing platelet units.

As described in Example 3, these platelet donations may be subjected to a pathogen inactivation process prior to combining, or alternatively after combining by subjecting the pooled platelet preparation to the pathogen inactivation process.

### Example 5: Preparing Platelet Units from Inventory and New Donations

The present disclosure also provides further improved methods for preparing a plurality of platelet units suitable for infusion. More specifically, in one embodiment, an inventory log of an inventory of apheresis-collected platelet preparations is maintained. The following Table 3 illustrates a model inventory log of an inventory of such platelet preparations, and includes for each platelet preparation a unique identifier, a donor ABO blood type, a donation time and a quantity of platelets (×10¹¹) in the preparation.

TABLE 3
Apheresis platelet inventory

| Identifier | ABO | Date | Time | Pre-count | Platelets | Products |
|---|---|---|---|---|---|---|
| 15072347 | B | 23July2015 | 16:33 | 375 | 10.96 | 3 |
| 15072348 | O | 23July2015 | 16:42 | 257 | 7.20 | 2 |
| 15072349 | O | 23July2015 | 16:55 | 246 | 6.38 | 2 |
| 15072201 | A | 22July2015 | 10:42 | 433 | 10.50 | 3 |
| 15072202 | AB | 22July2015 | 10:58 | 309 | 7.80 | 2 |
| 15072203 | A | 22July2015 | 11:26 | 309 | 8.75 | 2 |
| 15072204 | B | 22July2015 | 11:32 | 205 | 5.21 | 1 |
| 15072205 | O | 22July2015 | 11:48 | 441 | 7.00 | 2 |
| 15072206 | AB | 22July2015 | 12:14 | 220 | 4.50 | 1 |
| 15072207 | A | 22July2015 | 12:22 | 234 | 8.00 | 2 |
| 15072208 | A | 22July2015 | 12:59 | 190 | 4.86 | 1 |
| 15072209 | O | 22July2015 | 13:16 | 362 | 9.15 | 2 |

An available donor of blood type A suitable for donating platelets by apheresis is identified and a platelet preparation in the inventory (#15072208, an extra-large single) is selected from the inventory log for combining with platelets collected from the available donor. A new platelet donation with target yield of 5.10×10¹¹ platelets (an extra-large single) is collected by apheresis from the available donor and combined with the selected platelet preparation (e.g., within 6 hours) to yield a pooled platelet preparation comprising about 9.86×10¹¹ platelets. The pooled platelet preparation is separated into three platelet units, which is a greater number of platelet units than the two platelet units that can be prepared from selected platelet preparation and the new platelet donation without combining them as provided by the present disclosure. Similarly additional new platelet donations may be collected and combined with other platelet preparations, for example a new extra-large single platelet donation of blood type A to combine with inventory platelet preparation 15072203 and yield a pooled platelet preparation comprising sufficient platelets to generate 4 platelet units, a new extra-large double platelet donation of blood type B to combine with inventory platelet preparation 15072204 and yield a pooled platelet preparation comprising sufficient platelets to generate 4 platelet units, and a new extra-large double platelet donation of blood type O to combine with inventory platelet preparation 15072209 yield a pooled platelet preparation comprising sufficient platelets to generate 5 platelet units, to further increase the plurality of platelet units that may be obtained. For example, in the above scenario illustrated in this example, the 23 possible units from inventory may be combined with the four new platelet donations which would otherwise provide 6 new platelet units by conventional methods, to instead prepare 33 total platelet units (e.g., at least 3×10¹¹ per unit) based on the methods of the present disclosure. A pathogen inactivation process, such as described in Example 3 (e.g., photochemical pathogen-inactivation with amotosalen and UVA, and processing set as provided FIGS. 7 and 8), may optionally be performed prior to combining, or alternatively after combining by subjecting the pooled platelet preparation to the pathogen inactivation process.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Wherever an open-ended term is used to describe a feature or element, it is specifically contemplated that a closed-ended term can be used in place of the open-ended term without departing from the spirit and scope of the disclosure. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the description and does not pose a limitation on the scope of the description unless otherwise claimed. No language in the specification should be construed as indicating any non-claimed element as essential to the practice of the methods, systems and compositions disclosed herein.

Preferred embodiments are described herein. Variations of those preferred embodiments may become apparent to those working in the art upon reading the foregoing description. It is expected that skilled artisans will be able to employ such variations as appropriate, and the practice of the methods, systems and compositions described herein otherwise than as specifically described herein. Accordingly, the methods, systems and compositions described herein include all modifications and equivalents of the subject matter recited in the claims appended hereto as permitted by applicable law. Moreover, any combination of the above-described elements in all possible variations thereof is encompassed by the description unless otherwise indicated herein or otherwise clearly contradicted by context.

### LIST OF EMBODIMENTS

### Embodiment 1

A method of preparing a plurality of platelet units suitable for infusion, comprising:
a) collecting a first platelet donation by apheresis from a first donor,
b) collecting a second platelet donation by apheresis from a second donor,
c) combining the first platelet donation and the second platelet donation to yield a pooled platelet preparation, and
d) separating the pooled platelet preparation into a plurality of platelet units each in an individual container, wherein the plurality of platelet units comprises a greater number of platelet units than the total number of platelet units that can be prepared from the first platelet donation and second platelet donation without combining the first and second platelet donations.

### Embodiment 2

The method of embodiment 1, wherein collecting the first platelet donation from the first donor comprises targeting the platelet collection to yield a first platelet donation comprising less than about 98% of the number of platelets required to prepare one platelet unit.

### Embodiment 3

The method of embodiment 1, wherein the first platelet donation comprises less than the number of platelets required to prepare one platelet unit.

### Embodiment 4

The method of embodiment 1, wherein collecting the first platelet donation from the first donor comprises targeting the platelet collection to yield a first platelet donation comprising at least about 125% of the number of platelets required to prepare one platelet unit, but less than the number of platelets required to prepare two platelet units.

### Embodiment 5

The method of embodiment 1, wherein the first platelet donation comprises greater than the number of platelets required to prepare one platelet unit, but less than the number of platelets required to prepare two platelet units.

### Embodiment 6

The method of embodiment 5, wherein the first platelet donation comprises about 125% to about 198% of the number of platelets required to prepare one platelet unit.

### Embodiment 7

The method of embodiment 1, wherein collecting the first platelet donation from the first donor comprises targeting the platelet collection to yield a first platelet donation comprising at least about 225% of the number of platelets required to prepare one platelet unit, but less than the number of platelets required to prepare three platelet units.

### Embodiment 8

The method of embodiment 1, wherein the first platelet donation comprises greater than the number of platelets required to prepare two platelet units, but less than the number of platelets required to prepare three platelet units.

### Embodiment 9

The method of embodiment 8, wherein the first platelet donation comprises about 125% to about 198% of the number of platelets required to prepare two platelet units.

### Embodiment 10

The method of embodiment 8, wherein the first platelet donation comprises about 225% to about 298% of the number of platelets required to prepare one platelet unit.

### Embodiment 11

The method of embodiment 1, wherein collecting the first platelet donation from the first donor comprises targeting the platelet collection to yield a first platelet donation comprising at least about 325% of the number of platelets required to prepare one platelet unit, but less than the number of platelets required to prepare four platelet units.

### Embodiment 12

The method of embodiment 1, wherein the first platelet donation comprises greater than the number of platelets required to prepare three platelet units, but less than the number of platelets required to prepare four platelet units.

### Embodiment 13

The method of embodiment 12, wherein the first platelet donation comprises about 125% to about 198% of the number of platelets required to prepare three platelet units.

### Embodiment 14

The method of embodiment 12, wherein the first platelet donation comprises about 325% to about 398% of the number of platelets required to prepare one platelet unit.

### Embodiment 15

The method of any one of embodiments 1-14, wherein collecting the second platelet donation from the second donor comprises targeting the platelet collection to yield a second platelet donation comprising at least about 125% of the number of platelets required to prepare one platelet unit, but less than the number of platelets required to prepare two platelet units.

### Embodiment 16

The method of any one of embodiments 1-14, wherein the second platelet donation comprises greater than the number of platelets required to prepare one platelet unit, but less than the number of platelets required to prepare two platelet units.

### Embodiment 17

The method of embodiment 16, wherein the second platelet donation comprises about 125% to about 198% of the number of platelets required to prepare one platelet unit.

### Embodiment 18

The method of any one of embodiments 1-14, wherein collecting the second platelet donation from the second donor comprises targeting the platelet collection to yield a second platelet donation comprising at least about 225% of the number of platelets required to prepare one platelet unit, but less than the number of platelets required to prepare three platelet units.

### Embodiment 19

The method of any one of embodiments 1-14, wherein the second platelet donation comprises greater than the number of platelets required to prepare two platelet units, but less than the number of platelets required to prepare three platelet units.

### Embodiment 20

The method of embodiment 19, wherein the second platelet donation comprises about 125% to about 198% of the number of platelets required to prepare two platelet units.

### Embodiment 21

The method of embodiment 19, wherein the second platelet donation comprises about 225% to about 298% of the number of platelets required to prepare one platelet unit.

### Embodiment 22

The method of any one of embodiments 1-14, wherein collecting the second platelet donation from the second donor comprises targeting the platelet collection to yield a second platelet donation comprising at least about 325% of the number of platelets required to prepare one platelet unit, but less than the number of platelets required to prepare four platelet units.

### Embodiment 23

The method of any one of embodiments 1-14, wherein the second platelet donation comprises greater than the number of platelets required to prepare three platelet units, but less than the number of platelets required to prepare four platelet units.

### Embodiment 24

The method of embodiment 23, wherein the second platelet donation comprises about 125% to about 198% of the number of platelets required to prepare three platelet units.

### Embodiment 25

The method of embodiment 23, wherein the second platelet donation comprises about 325% to about 398% of the number of platelets required to prepare one platelet unit.

### Embodiment 26

The method of embodiment 2 or embodiment 3, wherein collecting the second platelet donation from the second donor comprises targeting the platelet collection to yield a second platelet donation comprising a sufficient number of platelets to prepare two platelet units after combining the first and second platelet donations and separating the resulting pooled platelet preparation into a plurality of platelet units.

### Embodiment 27

The method of embodiment 2 or embodiment 3, wherein the second platelet donation comprises a sufficient number of platelets that, when combined with the first platelet donation, yields a pooled platelet preparation comprising a sufficient number of platelets to separate into two platelet units.

### Embodiment 28

The method of embodiment 2 or embodiment 3, wherein collecting the second platelet donation from the second donor comprises collecting a sufficient number of platelets to prepare two platelet units from the pooled preparation.

### Embodiment 29

The method of any one of embodiments 4-6, wherein collecting the second platelet donation from the second donor comprises targeting the platelet collection to yield a second platelet donation comprising a sufficient number of platelets to prepare three platelet units after combining the first and second platelet donations and separating the resulting pooled platelet preparation into a plurality of platelet units.

### Embodiment 30

The method of any one of embodiments 4-6, wherein the second platelet donation comprises a sufficient number of platelets that, when combined with the first platelet donation, yields a pooled platelet preparation comprising a sufficient number of platelets to separate into three platelet units.

### Embodiment 31

The method of any one of embodiments 4-6, wherein collecting the second platelet donation from the second donor comprises collecting a sufficient number of platelets to prepare three platelet units from the pooled preparation.

### Embodiment 32

The method of any one of embodiments 7-10, wherein collecting the second platelet donation from the second donor comprises targeting the platelet collection to yield a second platelet donation comprising a sufficient number of platelets to prepare five platelet units after combining the first and second platelet donations and separating the resulting pooled platelet preparation into a plurality of platelet units.

### Embodiment 33

The method of any one of embodiments 7-10, wherein the second platelet donation comprises a sufficient number of platelets that, when combined with the first platelet donation, yields a pooled platelet preparation comprising a sufficient number of platelets for separation into five platelet units.

### Embodiment 34

The method of any one of embodiments 7-10, wherein collecting the second platelet donation from the second donor comprises collecting a sufficient number of platelets to prepare five platelet units from the pooled preparation.

### Embodiment 35

The method of any one of embodiments 11-14, wherein collecting the second platelet donation from the second donor comprises targeting the platelet collection to yield a second platelet donation comprising a sufficient number of platelets to prepare seven platelet units after combining the first and second platelet donations and separating the resulting pooled platelet preparation into a plurality of platelet units.

### Embodiment 36

The method of any one of embodiments 11-14, wherein the second platelet donation comprises a sufficient number of platelets that, when combined with the first platelet donation, yields a pooled platelet preparation comprising a sufficient number of platelets to separate into seven platelet units.

### Embodiment 37

The method of any one of embodiments 11-14, wherein collecting the second platelet donation from the second donor comprises collecting a sufficient number of platelets to prepare seven platelet units from the pooled preparation.

### Embodiment 38

The method of any one of embodiments 15-17, wherein the first platelet donation comprises greater than the number of platelets required to prepare one platelet unit, but less than the number of platelets required to prepare two platelet units, and the second platelet donation comprises greater than the number of platelets required to prepare one platelet unit, but less than the number of platelets required to prepare two platelet units.

### Embodiment 39

The method of embodiment 38, wherein the pooled platelet preparation comprises a sufficient number of platelets to prepare three platelet units.

### Embodiment 40

The method of embodiment 38 or embodiment 39, wherein the plurality of platelet units comprises three platelet units.

### Embodiment 41

The method of any one of embodiments 18-21, wherein the first platelet donation comprises greater than the number of platelets required to prepare two platelet units, but less than the number of platelets required to prepare three platelet units, and the second platelet donation comprises greater than the number of platelets required to prepare two platelet units, but less than the number of platelets required to prepare three platelet units.

### Embodiment 42

The method of embodiment 41, wherein the pooled platelet preparation comprises a sufficient number of platelets to prepare five platelet units.

### Embodiment 43

The method of embodiment 41 or embodiment 42, wherein the plurality of platelet units comprises five platelet units.

### Embodiment 44

The method of any one of embodiments 1-43, further comprising collecting a third platelet donation by apheresis from a third donor, combining the third platelet donation with the first and the second platelet donations to yield a pooled platelet preparation, and separating the pooled platelet preparation into a plurality of platelet units each in an individual container, wherein the plurality of platelet units comprises a greater number of platelet units than the total number of platelet units that can be prepared from the first platelet donation, the second platelet donation and the third platelet donation without combining the first, second and third platelet donations.

### Embodiment 45

The method of any one of embodiments 1-44, wherein the second platelet donation is collected within 20 hours from the time the first platelet donation is collected.

### Embodiment 46

The method of embodiment 45, wherein the second platelet donation is collected within 12 hours from the time the first platelet donation is collected.

### Embodiment 47

The method of embodiment 46, wherein the second platelet donation is collected within 6 hours from the time the first platelet donation is collected.

### Embodiment 48

The method of embodiment 44, wherein the third platelet donation is collected within 12 hours from the time the first platelet donation is collected.

### Embodiment 49

The method of any one of embodiments 1-48, wherein the pooled platelet preparation comprises platelets only from donors of the same ABO blood type.

### Embodiment 50

The method of any one of embodiments 1-49, further comprising subjecting each of the first platelet donation and the second platelet donation to a pathogen inactivation process.

### Embodiment 51

The method of embodiment 44, further comprising subjecting each of the first platelet donation, the second platelet donation and the third platelet donation to a pathogen inactivation process.

### Embodiment 52

The method of any one of embodiments 1-49, further comprising subjecting the pooled platelet preparation to a pathogen inactivation process.

### Embodiment 53

The method of any one of embodiments 50-52, wherein subjecting the platelet donations or pooled platelet preparation to a pathogen inactivation process comprises treating the platelet donation or pooled platelet preparation with a pathogen inactivating compound.

### Embodiment 54

The method of embodiment 53, wherein the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540.

### Embodiment 55

The method of embodiment 54, wherein the pathogen inactivating compound is a psoralen.

### Embodiment 56

The method of embodiment 55, wherein the pathogen inactivating compound is amotosalen.

### Embodiment 57

The method of any one of embodiments 1-56, wherein the platelet units in the plurality of platelet units each comprise a therapeutic dose of platelets suitable for infusion into a human subject.

### Embodiment 58

The method of embodiment 57, wherein the therapeutic dose of platelets comprises a minimum number of platelets as defined by criteria of a governmental agency, regulatory agency, institution and/or accrediting organization.

### Embodiment 59

The method of any one of embodiments 1-58 wherein the platelet units each comprise at least about 2.4×10¹¹ platelets.

### Embodiment 60

The method of embodiment 59, wherein the platelet units each comprise at least about 2.6×10¹¹ platelets.

### Embodiment 61

The method of embodiment 60, wherein the platelet units each comprise at least about 3.0×10¹¹ platelets.

### Embodiment 62

The method of any one of embodiments 26-28, wherein the platelet units each comprise a therapeutic dose of at least about 3×10¹¹ platelets.

### Embodiment 63

The method of embodiment 62, wherein the pooled platelet preparation comprises at least about 6.2×10¹¹ platelets.

### Embodiment 64

The method of any one of embodiments 29-31, wherein the platelet units each comprise a therapeutic dose of at least about 3×10¹¹ platelets.

### Embodiment 65

The method of embodiment 65, wherein the pooled platelet preparation comprises at least about 9.3×10¹¹ platelets.

### Embodiment 66

The method of any one of embodiments 26-34, wherein the platelet units each comprise a therapeutic dose of at least about 3×10¹¹ platelets.

### Embodiment 67

The method of embodiment 66, wherein the pooled platelet preparation comprises at least about 15.5×10¹¹ platelets.

### Embodiment 68

The method of any one of embodiments 2, 4, 7, 11, 15, 18, 22, 26, 29, 32 and 35, wherein targeting the platelet collection comprises determining an optimal platelet collection.

### Embodiment 69

The method of any one of embodiments 2, 4, 7, 11, 15, 18, 22, 26, 29, 32 and 35, wherein targeting the platelet collection comprises providing an input to an apheresis system.

### Embodiment 70

The method of any one of embodiments 2, 4, 7, 11, 15, 18, 22, 26, 29, 32 and 35, wherein targeting the platelet collection comprises providing a donation volume to an apheresis system.

### Embodiment 71

The method of any one of embodiments 1-70, wherein the first platelet donation and the second platelet donation are combined in a single container of suitable size for the volume of the pooled platelet preparation.

### Embodiment 72

A method of preparing a plurality of platelet units suitable for infusion, comprising:
a) maintaining an inventory log of an inventory of apheresis-collected platelet preparations, the inventory log comprising for each platelet preparation a unique identifier, a donor blood type, a donation time and a quantity of platelets in the preparation,
b) identifying an available donor suitable for donating platelets by apheresis,
c) selecting from the inventory log at least one platelet preparation in the inventory for combining with platelets collected from the available donor,
d) collecting a new platelet donation by apheresis from the available donor,
e) combining the at least one selected platelet preparation and the new platelet donation to yield a pooled platelet preparation, and
f) separating the pooled platelet preparation into a plurality of platelet units each in an individual container, wherein the plurality of platelet units comprises a greater number of platelet units than the total number of platelet units that can be prepared from the at least one selected platelet preparation and the new platelet donation without combining the at least one selected platelet preparation and new platelet donation.

### Embodiment 73

The method of embodiment 72, wherein the inventory of apheresis-collected platelet preparations comprises existing platelet preparations that have already been collected.

### Embodiment 74

The method of embodiment 72, wherein the inventory of apheresis-collected platelet preparations comprises one or more platelet preparations being collected by apheresis from one or more present donors, wherein the one or more present donors are not the available donor.

### Embodiment 75

The method of any one of embodiments 72-74, wherein the at least one selected platelet preparation comprises less than the number of platelets required to prepare one platelet unit.

### Embodiment 76

The method of any one of embodiment 72-74, wherein the at least one selected platelet preparation comprises greater than the number of platelets required to prepare one platelet unit, but less than the number of platelets required to prepare two platelet units.

### Embodiment 77

The method of any one of embodiments 72-74 and 76, wherein the at least one selected platelet preparation comprises at least about 125% of the number of platelets required to prepare one platelet unit, but less than the number of platelets required to prepare two platelet units.

### Embodiment 78

The method of embodiment 76, wherein the at least one selected platelet preparation comprises about 125% to about 198% of the number of platelets required to prepare one platelet unit.

### Embodiment 79

The method of any one of embodiments 72-74, wherein the at least one selected platelet preparation comprises greater than the number of platelets required to prepare two platelet units, but less than the number of platelets required to prepare three platelet units.

### Embodiment 80

The method of any one of embodiments 72-74 and 79, wherein the at least one selected platelet preparation comprises at least about 225% of the number of platelets required to prepare one platelet unit, but less than the number of platelets required to prepare three platelet units.

### Embodiment 81

The method of embodiment 80, wherein the at least one selected platelet preparation comprises about 225% to about 298% of the number of platelets required to prepare one platelet unit.

### Embodiment 82

The method of embodiment 80, wherein the at least one selected platelet preparation comprises about 125% to about 198% of the number of platelets required to prepare two platelet units.

### Embodiment 83

The method of any one of embodiments 72-74, wherein the at least one selected platelet preparation comprises greater than the number of platelets required to prepare three platelet units, but less than the number of platelets required to prepare four platelet units.

### Embodiment 84

The method of any one of embodiments 72-74 and 83, wherein the at least one selected platelet preparation comprises at least about 325% of the number of platelets required to prepare one platelet unit, but less than the number of platelets required to prepare four platelet units.

### Embodiment 85

The method of embodiment 84, wherein the at least one selected platelet preparation comprises about 325% to about 398% of the number of platelets required to prepare one platelet unit.

### Embodiment 86

The method of embodiment 83, wherein the at least one selected platelet preparation comprises about 125% to about 198% of the number of platelets required to prepare three platelet units.

### Embodiment 87

The method of any one of embodiments 72-86, wherein collecting the new platelet donation comprises targeting the platelet collection to yield a donation comprising at least about 125% of the number of platelets required to prepare one platelet unit, but less than the number of platelets required to prepare two platelet units.

### Embodiment 88

The method of any one of embodiments 72-86, wherein the new platelet donation comprises greater than the number of platelets required to prepare one platelet unit, but less than the number of platelets required to prepare two platelet units.

### Embodiment 89

The method of embodiment 88, wherein the new platelet donation comprises about 125% to about 198% of the number of platelets required to prepare one platelet unit.

### Embodiment 90

The method of any one of embodiments 72-86, wherein collecting the new platelet donation comprises targeting the platelet collection to yield a donation comprising at least about 225% of the number of platelets required to prepare one platelet unit, but less than the number of platelets required to prepare three platelet units.

### Embodiment 91

The method of any one of embodiments 72-86, wherein the new platelet donation comprises greater than the number of platelets required to prepare two platelet units, but less than the number of platelets required to prepare three platelet units.

### Embodiment 92

The method of embodiment 91, wherein the new platelet donation comprises about 125% to about 198% of the number of platelets required to prepare two platelet units.

### Embodiment 93

The method of embodiment 91, wherein the new platelet donation comprises about 225% to about 298% of the number of platelets required to prepare one platelet unit.

### Embodiment 94

The method of any one of embodiments 72-86, wherein collecting the new platelet donation comprises targeting the platelet collection to yield a donation comprising at least about 325% of the number of platelets required to prepare one platelet unit, but less than the number of platelets required to prepare four platelet units.

### Embodiment 95

The method of any one of embodiments 72-86, wherein the new platelet donation comprises greater than the number of platelets required to prepare three platelet units, but less than the number of platelets required to prepare four platelet units.

### Embodiment 96

The method of embodiment 95, wherein the new platelet donation comprises about 125% to about 198% of the number of platelets required to prepare three platelet units.

### Embodiment 97

The method of embodiment 95, wherein the new platelet donation comprises about 325% to about 398% of the number of platelets required to prepare one platelet unit.

### Embodiment 98

The method of embodiment 75, wherein collecting the new platelet donation from the available donor comprises targeting the platelet collection to yield a donation comprising a sufficient number of platelets to prepare two platelet units after combining the at least one selected platelet preparation and the new platelet donation and separating the resulting pooled platelet preparation into a plurality of platelet units.

### Embodiment 99

The method of embodiment 75, wherein the new platelet donation comprises a sufficient number of platelets that, when combined with the at least one selected platelet preparation, yields a pooled platelet preparation comprising a sufficient number of platelets to separate into two platelet units.

### Embodiment 100

The method of embodiment 75, wherein collecting the new platelet donation from the available donor comprises collecting a sufficient number of platelets to prepare two platelet units from the pooled preparation.

### Embodiment 101

The method of any one of embodiments 76-78, wherein collecting the new platelet donation from the available donor comprises targeting the platelet collection to yield a donation comprising a sufficient number of platelets to prepare three platelet units after combining the at least one selected platelet preparation and the new platelet donation and separating the resulting pooled platelet preparation into a plurality of platelet units.

### Embodiment 102

The method of any one of embodiments 76-78, wherein the new platelet donation comprises a sufficient number of platelets that, when combined with the at least one selected platelet preparation, yields a pooled platelet preparation comprising a sufficient number of platelets to separate into three platelet units.

### Embodiment 103

The method of any one of embodiments 76-78, wherein collecting the new platelet donation from the available donor comprises collecting a sufficient number of platelets to prepare three platelet units from the pooled preparation.

### Embodiment 104

The method of any one of embodiments 79-82, wherein collecting the new platelet donation from the available donor comprises targeting the platelet collection to yield a donation comprising a sufficient number of platelets to prepare five platelet units after combining the at least one selected platelet preparation and the new platelet donation and separating the resulting pooled platelet preparation into a plurality of platelet units.

### Embodiment 105

The method of any one of embodiments 79-82, wherein the new platelet donation comprises a sufficient number of platelets that, when combined with the at least one selected platelet preparation, yields a pooled platelet preparation comprising a sufficient number of platelets for separation into five platelet units.

### Embodiment 106

The method of any one of embodiments 79-82, wherein collecting the new platelet donation from the available donor comprises collecting a sufficient number of platelets to prepare five platelet units from the pooled preparation.

### Embodiment 107

The method of any one of embodiments 83-86, wherein collecting the new platelet donation from the available donor comprises targeting the platelet collection to yield a donation comprising a sufficient number of platelets to prepare seven platelet units after combining the at least one selected platelet preparation and the new platelet donations and separating the resulting pooled platelet preparation into a plurality of platelet units.

### Embodiment 108

The method of any one of embodiments 83-86, wherein the new platelet donation comprises a sufficient number of platelets that, when combined with the at least one selected platelet preparation, yields a pooled platelet preparation comprising a sufficient number of platelets to separate into seven platelet units.

### Embodiment 109

The method of any one of embodiments 83-86, wherein collecting the new platelet donation from the available donor comprises collecting a sufficient number of platelets to prepare seven platelet units from the pooled preparation.

### Embodiment 110

The method of embodiment 88 or embodiment 89, wherein the at least one selected platelet preparation comprises greater than the number of platelets required to prepare one platelet unit, but less than the number of platelets required to prepare two platelet units, and the new platelet donation comprises greater than the number of platelets required to prepare one platelet unit, but less than the number of platelets required to prepare two platelet units.

### Embodiment 111

The method of embodiment 110, wherein the pooled platelet preparation comprises a sufficient number of platelets to prepare three platelet units.

### Embodiment 112

The method of embodiment 110 or embodiment 111, wherein the plurality of platelet units comprises three platelet units.

### Embodiment 113

The method of any one of embodiments 91-93, wherein the at least one selected platelet preparation comprises greater than the number of platelets required to prepare two platelet units, but less than the number of platelets required to prepare three platelet units, and the new platelet donation comprises greater than the number of platelets required to prepare two platelet units, but less than the number of platelets required to prepare three platelet units.

### Embodiment 114

The method of embodiment 113, wherein the pooled platelet preparation comprises a sufficient number of platelets to prepare five platelet units.

### Embodiment 115

The method of embodiment 113 or embodiment 114, wherein the plurality of platelet units comprises five platelet units.

### Embodiment 116

The method of any one of embodiments 72-115, further comprising identifying a second available donor suitable for donating platelets by apheresis, collecting a second new platelet donation by apheresis from the second available donor, combining the second new platelet donation with the at least one selected platelet preparation and the new platelet donation to yield the pooled platelet preparation, and separating the pooled platelet preparation into a plurality of platelet units each in an individual container, wherein the plurality of platelet units comprises a greater number of platelet units than the total number of platelet units that can be prepared from the at least one selected platelet preparation, the new platelet donation and the second new platelet donation without combining the at least one selected platelet preparation, the new platelet donation and the second new platelet donation.

### Embodiment 117

The method of any one of embodiments 72-116, wherein the new platelet donation is collected within 12 hours from the time the at least one selected platelet preparation is collected.

### Embodiment 118

The method of embodiment 117, wherein the second new platelet donation is collected within 12 hours from the time the at least one selected platelet preparation and the new platelet donation are collected.

### Embodiment 119

The method of any one of embodiments 72-118, wherein the pooled platelet preparation comprises platelets only from donors of the same ABO blood type.

### Embodiment 120

The method of any one of embodiments 72-119, further comprising subjecting each of the at least one selected platelet preparation and the new platelet donation to a pathogen inactivation process.

### Embodiment 121

The method of embodiment 116, further comprising subjecting each of the at least one selected platelet preparation, the new platelet donation and the second new platelet donation to a pathogen inactivation process.

### Embodiment 122

The method of embodiment 120 or embodiment 121, wherein subjecting each of the at least one selected platelet preparation, the new platelet donation and the second new platelet donation to a pathogen inactivation process comprises treating each of the at least one selected platelet preparation, the new platelet donation and the second new platelet donation with a pathogen inactivating compound.

### Embodiment 123

The method of any one of embodiments 72-119, further comprising subjecting the pooled platelet preparation to a pathogen inactivation process.

### Embodiment 124

The method of embodiment 123, wherein subjecting the pooled platelet preparation to a pathogen inactivation process comprises treating the pooled platelet preparation with a pathogen inactivating compound.

### Embodiment 125

The method of embodiment 122 or embodiment 124, wherein the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540.

### Embodiment 126

The method of embodiment 125, wherein the pathogen inactivating compound is a psoralen.

### Embodiment 127

The method of embodiment 126, wherein the pathogen inactivating compound is amotosalen.

### Embodiment 128

The method of any one of embodiments 72-127, wherein the platelet units in the plurality of platelet units each comprise a therapeutic dose of platelets suitable for infusion into a human subject.

### Embodiment 129

The method of embodiment 128, wherein the therapeutic dose of platelets comprises a minimum number of platelets as defined by criteria of a governmental agency, regulatory agency, institution and/or accrediting organization.

### Embodiment 130

The method of any one of embodiments 72-129, wherein the platelet units each comprise at least about 2.4×10¹¹ platelets.

### Embodiment 131

The method of embodiment 130, wherein the platelet units each comprise at least about 2.6×10¹¹ platelets.

### Embodiment 132

The method of embodiment 131, wherein the platelet units each comprise at least about 3.0×10¹¹ platelets.

### Embodiment 133

The method of any one of embodiments 98-100, wherein the platelet units each comprise a therapeutic dose of at least about 3×10¹¹ platelets.

### Embodiment 134

The method of embodiment 133, wherein the pooled platelet preparation comprises at least about 6.2×10¹¹ platelets.

### Embodiment 135

The method of any one of embodiments 101-103, wherein the platelet units each comprise a therapeutic dose of at least about 3×10¹¹ platelets.

### Embodiment 136

The method of embodiment 135, wherein the pooled platelet preparation comprises at least about 9.3×10¹¹ platelets.

### Embodiment 137

The method of any one of embodiments 98-106, wherein the platelet units each comprise a therapeutic dose of at least about 3×10¹¹ platelets.

### Embodiment 138

The method of embodiment 137, wherein the pooled platelet preparation comprises at least about 15.5×10¹¹ platelets.

### Embodiment 139

The method of any one of embodiments 87, 90, 94, 98, 101, 104, 107, wherein targeting the platelet collection comprises determining an optimal platelet collection.

### Embodiment 140

The method of any one of embodiments 87, 90, 94, 98, 101, 104, 107, wherein targeting the platelet collection comprises providing an input to an apheresis system.

### Embodiment 141

The method of any one of embodiments 87, 90, 94, 98, 101, 104, 107, wherein targeting the platelet collection comprises providing a donation volume to an apheresis system.

### Embodiment 142

The method of any one of embodiments 72-141, wherein the at least one selected platelet preparation and the new platelet donation are combined in a single container of suitable size for the volume of the pooled platelet preparation.

### Embodiment 143

A platelet unit suitable for infusion prepared by the method of any one of embodiments 1-143.

### Embodiment 144

A method of preparing two or more therapeutic dosage units of platelets comprising:
a) combining two or more apheresis-derived platelet donor units to form a pooled platelet composition,
b) subjecting the pooled platelet composition to a pathogen inactivation process, and
c) separating the pooled platelet composition into two or more therapeutic dosage units of platelets, each in an individual container.

### Embodiment 145

The method of embodiment 144, wherein the number of therapeutic dosage units of platelets following step c) is greater than the number of therapeutic dosage units of platelets that can be prepared from the two or more apheresis-derived platelet donor units individually, without combining the two or more donor units.

### Embodiment 146

The method of embodiment 144 or embodiment 145, wherein two apheresis-derived platelet donor units are combined and each donor unit comprises greater than the number of platelets required to prepare one therapeutic dosage unit of platelets, but less than the number of platelets required to prepare two therapeutic dosage units of platelets.

### Embodiment 147

The method of embodiment 146, wherein each donor unit comprises at least about 125% of the number of platelets required to prepare one platelet unit.

### Embodiment 148

The method of embodiment 144 or embodiment 145, wherein the two apheresis-derived platelet donor units are combined and each donor unit comprises greater than the number of platelets required to prepare two therapeutic dosage units of platelets, but less than the number of platelets required to prepare three therapeutic dosage units of platelets.

### Embodiment 149

The method of embodiment 148, wherein each donor unit comprises at least about 225% of the number of platelets required to prepare one platelet unit.

### Embodiment 150

The method of any one of embodiments 144-149, wherein at least one of two or more apheresis-derived platelet donor units is selected from an inventory log of an inventory of apheresis-collected platelet donor units, the inventory log comprising for each platelet donor unit a unique identifier, a donor blood type, a donation time and a quantity of platelets in the donor unit.

### Embodiment 151

The method of embodiment 150, wherein each of the two or more apheresis-derived platelet donor units is selected from the inventory log.

### Embodiment 152

The method of any one of embodiments 144-151, wherein the two or more apheresis-derived platelet donor units are combined within 12 hours from the time of collection.

### Embodiment 153

The method of any one of embodiments 144-151, wherein the two or more apheresis-derived platelet donor units are from donors of the same ABO blood type.

### Embodiment 154

The method of any one of embodiments 144-153, wherein subjecting the pooled platelet composition to a pathogen inactivation process comprises treating the pooled pathogen composition with a pathogen inactivating compound, Embodiment 155. The method of embodiment 154, wherein the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540.

### Embodiment 156

The method of embodiment 155, wherein the pathogen inactivating compound is a psoralen.

### Embodiment 157

The method of embodiment 156, wherein the pathogen inactivating compound is amotosalen.

### Embodiment 158

The method of any one of embodiments 144-157, wherein each therapeutic dosage unit of platelets comprises at least 2.4×10¹¹ platelets.

### Embodiment 159

The method of embodiment 158, wherein each therapeutic dosage units of platelets comprises at least 2.6×10¹¹ platelets.

### Embodiment 160

The method of embodiment 159, wherein each therapeutic dosage units of platelets comprises at least 3.0×10¹¹ platelets.

### Embodiment 161

A therapeutic dosage unit of platelets prepared by the method of any one of embodiments 144-160.

### Embodiment 162

A method for collecting a platelet donation by apheresis from a donor, comprising:
a) maintaining an inventory log of an inventory of apheresis-collected platelet preparations, the inventory log comprising for each platelet preparation a unique identifier, a donor blood type, a donation date and a quantity of platelets in the preparation,
b) identifying a donor suitable for donating platelets by apheresis,
c) selecting from the inventory log one or more platelet preparations suitable for combining with a new platelet donation to be collected by apheresis from the donor,
d) determining a target volume of the new platelet donation, wherein the target volume optimizes the number of platelet units that can be prepared from a combination comprising the new platelet donation and the one or more selected platelet preparations; and
e) collecting the target volume of the new platelet donation by apheresis from the donor.

### Embodiment 163

The method of embodiment 162, further comprising:

f) combining the one or more selected platelet preparations and the new platelet donation to yield a pooled platelet preparation; and

g) separating the pooled platelet preparation into a plurality of platelet units each in an individual container.

### Embodiment 164

The method of embodiment 163, wherein each of the platelet units in the plurality of platelet units comprises a therapeutic dose suitable for infusion into a human subject.

### Embodiment 165

The method of embodiment 163 or embodiment 164, wherein the plurality of platelet units comprises a greater number of platelet units than the total number of platelet units that can be prepared from the one or more selected platelet preparations and the new platelet donation without combining the one or more selected platelet preparations and the new platelet donation.

### Embodiment 166

The method of any one of embodiments 162-165, wherein the one or more selected platelet preparations comprises less than the number of platelets required to prepare one platelet unit.

### Embodiment 167

The method of embodiment 166, wherein the target volume of the new platelet donation is a volume comprising a sufficient number of platelets to prepare two platelet units after combining the one or more selected platelet preparations and the new platelet donation and separating the resulting pooled platelet preparation into a plurality of platelet units.

### Embodiment 168

The method of embodiment 166, wherein the new platelet donation comprises a sufficient number of platelets that, when combined with the one or more selected platelet preparation, yields a pooled platelet preparation comprising a sufficient number of platelets to separate into two platelet units.

### Embodiment 169

The method of embodiment 166, wherein collecting the new platelet donation from the donor comprises collecting a sufficient number of platelets to prepare two platelet units from the pooled preparation.

### Embodiment 170

The method of any one of embodiments 162-165, wherein the one or more selected platelet preparations comprises greater than the number of platelets required to prepare one platelet unit, but less than the number of platelets required to prepare two platelet units.

### Embodiment 171

The method of embodiment 167, wherein the one or more selected platelet preparations comprises at least about 125% of the number of platelets required to prepare one platelet unit, but less than the number of platelets required to prepare two platelet units.

### Embodiment 172

The method of embodiment 170 or embodiment 171, wherein the target volume of the new platelet donation is a volume comprising a sufficient number of platelets to prepare three platelet units after combining the one or more selected platelet preparations and the new platelet donation and separating the resulting pooled platelet preparation into a plurality of platelet units.

### Embodiment 173

The method of embodiment 170 or embodiment 171, wherein the new platelet donation comprises a sufficient number of platelets that, when combined with the one or more selected platelet preparations, yields a pooled platelet preparation comprising a sufficient number of platelets to separate into three platelet units.

### Embodiment 174

The method of embodiment 170 or embodiment 171, wherein collecting the new platelet donation from the donor comprises collecting a sufficient number of platelets to prepare three platelet units from the pooled preparation.

### Embodiment 175

The method of any one of embodiments 162-165, wherein the one or more selected platelet preparations comprises greater than the number of platelets required to prepare two platelet units, but less than the number of platelets required to prepare three platelet units.

### Embodiment 176

The method of embodiment 175, wherein the one or more selected platelet preparations comprises at least about 225% of the number of platelets required to prepare one platelet unit, but less than the number of platelets required to prepare three platelet units.

### Embodiment 177

The method of embodiment 175 or embodiment 176, wherein the target volume of the new platelet donation is a volume comprising a sufficient number of platelets to prepare five platelet units after combining the one or more selected platelet preparations and the new platelet donation and separating the resulting pooled platelet preparation into a plurality of platelet units.

### Embodiment 178

The method of embodiment 175 or embodiment 176, wherein the new platelet donation comprises a sufficient number of platelets that, when combined with the one or more selected platelet preparation, yields a pooled platelet preparation comprising a sufficient number of platelets to separate into five platelet units.

### Embodiment 179

The method of embodiment 175 or embodiment 176, wherein collecting the new platelet donation from the donor comprises collecting a sufficient number of platelets to prepare five platelet units from the pooled preparation.

### Embodiment 180

The method of embodiment 164 or embodiment 165, wherein each of the one or more selected platelet preparations and the new platelet donation comprises greater than the number of platelets required to prepare one therapeutic dose, but less than the number of platelets required to prepare two therapeutic doses.

### Embodiment 181

The method of embodiment 164 or embodiment 165, wherein each of the one or more selected platelet preparations and the new platelet donation comprises greater than the number of platelets required to prepare two therapeutic dosage units of platelets, but less than the number of platelets required to prepare three therapeutic dosage units of platelets.

### Embodiment 182

The method of any one of embodiments 163-182, wherein the one or more selected platelet preparations and the new platelet donation are combined within 12 hours from the time of collection.

### Embodiment 183

The method of any one of embodiments 163-182, wherein the one or more selected platelet preparations and the new platelet donation are from donors of the same ABO blood type.

### Embodiment 184

The method of any one of embodiments 163-182, wherein each platelet unit comprises at least 2.4×10¹¹ platelets.

### Embodiment 185

The method of embodiment 184, wherein each platelet unit comprises at least 2.6×10¹¹ platelets.

### Embodiment 186

The method of embodiment 185, wherein each platelet unit comprises at least 3.0×10¹¹ platelets.

### Embodiment 187

The method of any one of embodiments 163-186, further comprising subjecting the pooled platelet preparation to a pathogen inactivation process.

### Embodiment 188

The method of embodiment 187, wherein subjecting the pooled platelet preparation to a pathogen inactivation process comprises treating the pooled platelet preparation with a pathogen inactivating compound.

### Embodiment 189

The method of any one of embodiments 163-186, further comprising subjecting each of the one or more selected platelet preparations and the new platelet donation to a pathogen inactivation process.

### Embodiment 190

The method of embodiment 189, wherein subjecting the one or more selected platelet preparations and the new platelet donation to a pathogen inactivation process comprises treating the one or more selected platelet preparations and the new platelet donation with a pathogen inactivating compound.

### Embodiment 191

The method of any one of embodiments 187-190, wherein the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540.

### Embodiment 192

The method of embodiment 191, wherein the pathogen inactivating compound is a psoralen.

### Embodiment 193

The method of embodiment 192, wherein the pathogen inactivating compound is amotosalen.

### Embodiment 194

A platelet unit suitable for infusion into a human subject, wherein the platelet unit is selected from a plurality of platelet units prepared by the method of any one of embodiments 163-193.

### Embodiment 195

A therapeutic dosage unit of platelets suitable for infusion into a subject, wherein the therapeutic dosage unit comprises pooled apheresis-derived platelets from two or more donors, and wherein the pooled apheresis-derived platelets have been treated with a pathogen inactivating compound.

### Embodiment 196

The therapeutic dosage unit of embodiment 195, wherein the apheresis-derived platelets have been treated with the pathogen inactivating compound prior to pooling.

### Embodiment 197

The therapeutic dosage unit of platelets of embodiment 195, wherein the apheresis-derived platelets have been treated with the pathogen inactivating compound after pooling.

### Embodiment 198

The therapeutic dosage unit of platelets of embodiments 195-197, wherein the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540.

### Embodiment 199

The therapeutic dosage unit of platelets of embodiment 198, wherein the pathogen inactivating compound is a psoralen.

### Embodiment 200

The therapeutic dosage unit of platelets of embodiment 199, wherein the pathogen inactivating compound is amotosalen.

### Embodiment 201

The therapeutic dosage unit of platelets of any one of embodiments 195-200, wherein the apheresis-derived platelets are from donors of the same ABO blood type.

### Embodiment 202

The therapeutic dosage unit of platelets of any one of embodiments 195-201, wherein the therapeutic dosage unit comprises at least 2.4×10¹¹ platelets.

### Embodiment 203

The therapeutic dosage unit of platelets of embodiment 202, wherein the therapeutic dosage unit comprises at least 2.6×10¹¹ platelets.

### Embodiment 204

The therapeutic dosage unit of platelets of embodiment 203, wherein the therapeutic dosage unit comprises at least 3.0×10¹¹ platelets.

### Embodiment 205

A method of infusing platelets into a subject in need thereof, comprising infusing into the subject a platelet unit of embodiment 143 or embodiment 194, or a therapeutic dosage unit of platelets of any one of embodiments 161 and 195-204.

### Embodiment 206

A computer-implemented method for optimization of platelet units for infusion, comprising:

at a computer system including one or more processors and memory, receiving one or more values associated with one or more properties of a platelet donation;

instructing to search, based on the received one or more values, a plurality of values associated with representations of a plurality of platelet preparations;

determining, based on the search results, a volume of the platelet donation; and

providing the determined volume of the platelet donation, wherein the determined volume of the platelet donation enables optimization of platelet units.

### Embodiment 207

The method of embodiment 206, wherein at least one of the platelet donation and the plurality of platelet preparations comprises apheresis-derived platelets.

### Embodiment 208

The method of embodiment 206 or 207, wherein the platelet donation comprises platelets being currently collected from a donor or platelets to be collected from a donor.

### Embodiment 209

The method of any one of embodiments 206-208, wherein the one or more properties of the platelet donation comprises: a blood type, a donation time, number of platelets per unit volume, and optionally a maximum platelet donation volume.

### Embodiment 210

The method of embodiment 209, wherein the value of the blood type includes one of: A, B, AB, or O.

### Embodiment 211

The method of any one of embodiments 206-210, wherein instructing to search the plurality of values associated with representations of a plurality of platelet preparations comprises:
obtaining one or more criteria for searching using the one or more received values associated with one or more properties of the platelet donation;
instructing to search the plurality of values associated with the representations of the plurality of platelet preparations based on the one or more criteria for searching; and
obtaining one or more candidate representations of platelet preparations from the representations of the plurality of platelet preparations, the one or more candidate representations of platelet preparations having values that satisfy the one or more criteria for searching.

### Embodiment 212

The method of embodiment 211, wherein the criteria for searching include one or more threshold conditions of one or more of: a blood type, a donation time, a platelet volume and number of platelets per unit volume.

### Embodiment 213

The method of embodiment 211 or 212, wherein obtaining the one or more candidate representations of platelet preparations comprises filtering the representations of the plurality of platelet preparations based on the one or more criteria for searching.

### Embodiment 214

The method of any one of embodiments 206-213, wherein the representations of the plurality of platelet preparations include at least one of: platelet preparations stored in a platelet preparation inventory, platelet preparations being currently collected from a plurality of donors, and platelet preparations to be collected from a plurality of donors.

### Embodiment 215

The method of any one of embodiments 206-214, wherein determining the volume of the platelet donation comprises:
obtaining one or more values corresponding to one or more candidate representations of platelet preparations provided by the search results;
obtaining one or more criteria for platelet units, the criteria for platelet units enabling optimization of platelet units;
determining, based on the one or more values corresponding to one or more candidate representations of platelet preparations and the one or more criteria for platelet units, one or more platelet preparations to select from the one or more candidate representations of the plurality of platelet preparations; and
obtaining the volume of the platelet donation based on the determined platelet preparation.

### Embodiment 216

The method of any one of embodiments 206-215, wherein providing the determined volume of the platelet donation comprises at least one of:
displaying the determined volume of the platelet donation on a display of the computer system;
sending a notification providing the determining volume of the platelet donation; and
providing the determined volume of the platelet donation to an apheresis system.

### Embodiment 217

The method of any one of embodiments 206-216, further comprising combining the platelet donation and one or more platelet preparations selected from the representations of the plurality of platelet preparations to yield a pooled platelet preparation.

### Embodiment 218

The method of embodiment 217, further comprising separating the pooled platelet preparation into a plurality of platelet units each in an individual container.

### Embodiment 219

A non-transitory computer readable medium having instructions stored thereon for optimization of platelet units for infusion, the instructions, when executed by one or more processors of a computer system, cause the computer system to:
receive one or more values associated with one or more properties of a platelet donation;
instruct to search, based on the received one or more values, a plurality of values associated with representations of a plurality of platelet preparations;
determine, based on the search results, a volume of the platelet donation; and
provide the determined volume of the platelet donation, wherein the determined volume of the platelet donation enables optimization of platelet units.

### Embodiment 220

A system for optimization of platelet units for infusion, comprising:
one or more processors; and
memory having instructions stored thereon, the instruction, when executed by the one or more processors, cause the computer system to:
receive one or more values associated with one or more properties of a platelet donation;
instruct to search, based on the received one or more values, a plurality of values associated with representations of a plurality of platelet preparations;
determine, based on the search results, a volume of the platelet donation; and
provide the determined volume of the platelet donation, wherein the determined volume of the platelet donation enables optimization of platelet units.

### Embodiment 221

The system of embodiment 220, wherein the computer system is integrated with an apheresis system.

### Embodiment 222

A computer-implemented method for optimization of platelet units for infusion, comprising:
at a computer system including one or more processors and memory,
receiving one or more values associated with one or more properties of a first platelet preparation;
instructing to search, based on the received one or more values, a plurality of values associated with representations of a plurality of platelet preparations;
selecting, based on the search results, a second platelet preparation from the representations of a plurality of platelet preparations; and
providing the selected second platelet preparation, wherein the selected second platelet preparation enables optimization of platelet units.

### Embodiment 223

The method of embodiment 222, wherein at least one of the first platelet preparation and the plurality of platelet preparations comprises apheresis-derived platelets.

### Embodiment 224

The method of embodiment 222 or 223, wherein the first platelet preparation comprises a platelet preparation stored in a platelet preparation inventory.

### Embodiment 225

The method of any one of embodiments 222-224, wherein the one or more properties of the first platelet preparation comprises: a blood type, a donation time, a volume, and number of platelets per unit volume.

### Embodiment 226

The method of embodiment 225, wherein the value of the blood type includes one of: A, B, AB, or O.

### Embodiment 227

The method of any one of embodiments 222-226, wherein instructing to search the plurality of values associated with representations of a plurality of platelet preparations comprises:
obtaining one or more criteria for searching using the one or more received values associated with one or more properties of the first platelet preparation;
instructing to search the plurality of values associated with the representations of the plurality of platelet preparations based on the one or more criteria for searching; and
obtaining one or more candidate representations of platelet preparations from the representations of the plurality of platelet preparations, the one or more candidate representations of platelet preparations having values that satisfy the one or more criteria for searching.

### Embodiment 228

The method of embodiment 227, wherein the criteria for searching include one or more threshold conditions of one or more of: a blood type, a donation time, a volume, and number of platelets per unit volume.

### Embodiment 229

The method of embodiment 227 or 228, wherein obtaining the one or more candidate representations of platelet preparations comprises filtering the representations of the plurality of platelet preparations based on the one or more criteria for searching.

### Embodiment 230

The method of any one of embodiments 222-229, wherein the representations of the plurality of platelet preparations include at least one of: platelet preparations stored in a platelet preparation inventory, platelet preparations being currently collected from a plurality of donors, and platelet preparations to be collected from a plurality of donors.

### Embodiment 231

The method of any one of embodiments 222-230, wherein selecting the second platelet preparation comprises:
obtaining one or more values corresponding to one or more candidate representations of platelet preparations provided by the search results;
obtaining one or more criteria for platelet units, the criteria for platelet units enabling optimization of platelet units; and
selecting, based on the one or more values corresponding to one or more candidate representations of platelet preparations and the one or more criteria for platelet units, a second platelet preparation from the one or more candidate representations of platelet preparations.

### Embodiment 232

The method of any one of embodiments 222-231, wherein providing the selected second platelet preparation comprises at least one of:
displaying the selected second platelet preparation on a display of the computer system; and
sending a notification providing the selected second platelet preparation; and
providing an identification of the selected second platelet preparation to an apheresis system.

### Embodiment 233

The method of any one of embodiments 222-232, further comprising combining the first platelet preparation and selected second platelet preparation to yield a pooled platelet preparation.

### Embodiment 234

The method of embodiment 233, further comprising separating the pooled platelet preparation into a plurality of platelet units each in an individual container.

### Embodiment 235

A non-transitory computer readable medium having instructions stored thereon for optimization of platelet units for infusion, the instructions, when executed by one or more processors of a computer system, cause the computer system to:
receive one or more values associated with one or more properties of a first platelet preparation;
instruct to search, based on the received one or more values, a plurality of values associated with representations of a plurality of platelet preparations;
select, based on the search results, a second platelet preparation from the representations of a plurality of platelet preparations; and
provide the selected second platelet preparation, wherein the selected second platelet preparation enables optimization of platelet units.

### Embodiment 236

A system for optimization of platelet units for infusion, comprising:
one or more processors; and
memory having instructions stored thereon, the instruction, when executed by the one or more processors, cause the computer system to:
receive one or more values associated with one or more properties of a first platelet preparation;
instruct to search, based on the received one or more values, a plurality of values associated with representations of a plurality of platelet preparations;
select, based on the search results, a second platelet preparation from the representations of a plurality of platelet preparations; and
provide the selected second platelet preparation, wherein the selected second platelet preparation enables optimization of platelet units.

### Embodiment 237

The system of embodiment 236, wherein the computer system is integrated with an apheresis system.

### Embodiment 238

The method of any one of embodiments 217, 218, 233 and 234, further comprising subjecting the pooled platelet preparation to a pathogen inactivation process.

### Embodiment 239

The method of embodiment 238, wherein subjecting the pooled platelet preparation to a pathogen inactivation process comprises treating the pooled platelet preparation with a pathogen inactivating compound.

### Embodiment 240

The method of embodiment 239, wherein the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540.

### Embodiment 241

The method of embodiment 240, wherein the pathogen inactivating compound is a psoralen.

### Embodiment 242

The method of embodiment 241, wherein the pathogen inactivating compound is amotosalen.

### Embodiment 243

A processing set for preparing a plurality of platelet units from two or more platelet donations, wherein each of the platelet units is pathogen-inactivated and suitable for infusion into a subject, the processing set comprising:
a) a first container that contains a pathogen inactivation compound (PIC) and is suitable for combining the two or more platelet donations with the PIC;
b) a second container, coupled to the first container, within which the two or more platelet donations in admixture with the PIC can be photochemically inactivated under sterile conditions, wherein the volume of the two or more platelet donations is sufficient to yield the plurality of platelet units;
c) a compound absorption device (CAD) coupled to the second container such that the two or more photochemically inactivated platelet donations can be transferred from the second container to the compound absorption device under sterile conditions; and
d) two or more third containers, each of which is coupled to the compound absorption device such that the two or more photochemically inactivated platelet donations can be transferred from the compound absorption device to the two or more third containers under sterile conditions to provide the plurality of platelet units,
wherein the plurality of platelet units comprises a greater number of platelet units than the total number of platelet units that can be prepared from the two or more donations without combining the two or more platelet donations.

### Embodiment 244

The processing set of embodiment 243, comprising three or more third containers, each of which is coupled to the compound absorption device such that the two or more photochemically inactivated platelet donations can be transferred from the compound absorption device to the three or more third containers under sterile conditions to provide the plurality of pathogen-inactivated platelet units.

### Embodiment 245

The processing set of embodiment 243 or embodiment 244, further comprising an additional container suitable for containing one or more of the two or more platelet donations, wherein the additional container is coupled to the first container such that the one or more platelet donations of the additional container can be transferred from the additional container to the first container under sterile conditions.

### Embodiment 246

The processing set of embodiment 245, wherein the additional container is suitable for containing a platelet donation obtained by apheresis.

### Embodiment 247

The processing set of embodiment 245 or embodiment 246, wherein the additional container is suitable for sterile coupling with a second additional container.

### Embodiment 248

The processing set of embodiment 247, wherein the second additional container contains one or more of the two or more platelet donations, and wherein the additional container is suitable for sterile coupling with the second additional container such that the one or more platelet donations of the second additional container can be transferred from the second additional container to the additional container under sterile conditions.

### Embodiment 249

The processing set of any one of embodiments 245-248, further comprising a second additional container suitable for containing one or more of the two or more platelet donations, wherein the second additional container is coupled to the additional container such that the one or more platelet donations of the second additional container can be transferred from the second additional container to the additional container under sterile conditions.

### Embodiment 250

The processing set of any one of embodiments 247-249, wherein the additional container is suitable for containing a platelet donation obtained by apheresis from a first donor, and wherein the second additional container is suitable for containing a platelet donation obtained by apheresis from a second donor.

### Embodiment 251

The processing set of any one of embodiments 243-250, wherein the PIC is a psoralen.

### Embodiment 252

The processing set of embodiment 251, wherein the PIC is amotosalen.

### Embodiment 253

A kit comprising (a) a processing set, and (b) instructions for using the processing set to prepare a plurality of platelet units from two or more platelet donations, wherein each of the platelet units is pathogen-inactivated and suitable for infusion into a subject, wherein the plurality of platelet units comprises a greater number of platelet units than can be prepared from the two or more donations without combining the two or more platelet donations, and
wherein the processing set comprises:
1) a first container that contains a pathogen inactivation compound (PIC) and is suitable for combining the two or more platelet donations with the PIC;
2) a second container, coupled to the first container, within which the two or more platelet donations in admixture with the PIC can be photochemically inactivated under sterile conditions, wherein the volume of the two or more platelet donations is sufficient to yield the plurality of platelet units;
3) a compound absorption device (CAD) coupled to the second container such that the two or more photochemically inactivated platelet donations can be transferred from the second container to the compound absorption device under sterile conditions; and
4) two or more third containers, each of which is coupled to the compound absorption device such that the two or more photochemically inactivated platelet donations can be transferred from the compound absorption device to the two or more third containers under sterile conditions to provide the plurality of platelet units.

### Embodiment 254

The kit of embodiment 253, wherein the processing set comprises three or more third containers, each of which is coupled to the compound absorption device such that the two or more photochemically inactivated platelet donations can be transferred from the compound absorption device to the three or more third containers under sterile conditions to provide the plurality of pathogen-inactivated platelet units.

### Embodiment 255

The kit of embodiment 253 or embodiment 254, wherein the processing set further comprises an additional container suitable for containing one or more of the two or more platelet donations, wherein the additional container is coupled to the first container such that the one or more platelet donations of the additional container can be transferred from the additional container to the first container under sterile conditions.

### Embodiment 256

The kit of embodiment 255, wherein the additional container is suitable for containing a platelet donation obtained by apheresis.

### Embodiment 257

The kit of embodiment 255 or 256, wherein the additional container is suitable for sterile coupling with a second additional container.

### Embodiment 258

The kit of embodiment 257, wherein the second additional container contains one or more of the two or more platelet donations, and wherein the additional container is suitable for sterile coupling with the second additional container such that the one or more platelet donations of the second additional container can be transferred from the second additional container to the additional container under sterile conditions.

### Embodiment 259

The kit of any one of embodiments 255-258, wherein the processing set further comprises a second additional container suitable for containing one or more of the two or more platelet donations, wherein the second additional container is coupled to the additional container such that the one or more platelet donations of the second additional container can be transferred from the second additional container to the additional container under sterile conditions.

### Embodiment 260

The kit of any one of embodiments 257-259, wherein the additional container is suitable for containing a platelet donation obtained by apheresis from a first donor, and wherein the second additional container is suitable for containing a platelet donation obtained by apheresis from a second donor.

### Embodiment 261

The kit of any one of embodiments 253-260, wherein the PIC is a psoralen.

### Embodiment 262

The kit of embodiment 261, wherein the PIC is amotosalen.

### Embodiment 263

The processing set of any one of embodiments 243-252 or the kit of any one of embodiments 253-262, wherein the platelet units in the plurality of platelet units each comprise a therapeutic dose of platelets.

What is claimed is:

1. A method of preparing a plurality of platelet units suitable for infusion, comprising:
a) collecting a first platelet donation by apheresis from a first donor,
b) collecting a second platelet donation by apheresis from a second donor,
c) combining the first platelet donation and the second platelet donation to yield a pooled platelet preparation, and
d) separating the pooled platelet preparation into a plurality of platelet units each in an individual container, wherein the plurality of platelet units comprises a greater number of platelet units than the total number of platelet units that can be prepared from the first platelet donation and second platelet donation without combining the first and second platelet donations.

2. The method of claim 1, wherein collecting the first platelet donation from the first donor comprises targeting the platelet collection to yield a first platelet donation comprising at least about 125% of the number of platelets required to prepare one platelet unit, but less than the number of platelets required to prepare two platelet units.

3. The method of claim 1, wherein the first platelet donation comprises greater than the number of platelets required to prepare one platelet unit, but less than the number of platelets required to prepare two platelet units.

4. The method of claim 1, wherein collecting the first platelet donation from the first donor comprises targeting the platelet collection to yield a first platelet donation comprising at least about 225% of the number of platelets required to prepare one platelet unit, but less than the number of platelets required to prepare three platelet units.

5. The method of claim 1, wherein the first platelet donation comprises greater than the number of platelets required to prepare two platelet units, but less than the number of platelets required to prepare three platelet units.

6. The method of claim 1, wherein collecting the first platelet donation from the first donor comprises targeting the platelet collection to yield a first platelet donation comprising at least about 325% of the number of platelets required to prepare one platelet unit, but less than the number of platelets required to prepare four platelet units.

7. The method of claim 1, wherein the first platelet donation comprises greater than the number of platelets required to prepare three platelet units, but less than the number of platelets required to prepare four platelet units.

8. The method of any one of claims 1-7, wherein collecting the second platelet donation from the second donor comprises targeting the platelet collection to yield a second platelet donation comprising at least about 125% of the number of platelets required to prepare one platelet unit, but less than the number of platelets required to prepare two platelet units.

9. The method of any one of claims 1-7, wherein the second platelet donation comprises greater than the number of platelets required to prepare one platelet unit, but less than the number of platelets required to prepare two platelet units.

10. The method of any one of claims 1-7, wherein collecting the second platelet donation from the second donor comprises targeting the platelet collection to yield a second platelet donation comprising at least about 225% of the number of platelets required to prepare one platelet unit, but less than the number of platelets required to prepare three platelet units.

11. The method of any one of claims 1-7, wherein the second platelet donation comprises greater than the number of platelets required to prepare two platelet units, but less than the number of platelets required to prepare three platelet units.

12. The method of any one of claims 1-7, wherein collecting the second platelet donation from the second donor comprises targeting the platelet collection to yield a second platelet donation comprising at least about 325% of the number of platelets required to prepare one platelet unit, but less than the number of platelets required to prepare four platelet units.

13. The method of claim 1, wherein collecting the second platelet donation from the second donor comprises collecting a sufficient number of platelets to prepare two platelet units from the pooled preparation.

14. The method of claim 2 or claim 3, wherein collecting the second platelet donation from the second donor comprises targeting the platelet collection to yield a second platelet donation comprising a sufficient number of platelets to prepare three platelet units after combining the first and second platelet donations and separating the resulting pooled platelet preparation into a plurality of platelet units.

15. The method of claim 2 or claim 3, wherein the second platelet donation comprises a sufficient number of platelets that, when combined with the first platelet donation, yields a pooled platelet preparation comprising a sufficient number of platelets to separate into three platelet units.

16. The method of claim 4 or claim 5, wherein collecting the second platelet donation from the second donor comprises targeting the platelet collection to yield a second platelet donation comprising a sufficient number of platelets to prepare five platelet units after combining the first and second platelet donations and separating the resulting pooled platelet preparation into a plurality of platelet units.

17. The method of claim 4 or claim 5, wherein the second platelet donation comprises a sufficient number of platelets that, when combined with the first platelet donation, yields a pooled platelet preparation comprising a sufficient number of platelets for separation into five platelet units.

18. The method of claim 6 or claim 7, wherein the second platelet donation comprises a sufficient number of platelets that, when combined with the first platelet donation, yields a pooled platelet preparation comprising a sufficient number of platelets to separate into seven platelet units.

19. The method of claim 8 or claim 9, wherein the first platelet donation comprises greater than the number of platelets required to prepare one platelet unit, but less than the number of platelets required to prepare two platelet units, and the second platelet donation comprises greater than the number of platelets required to prepare one platelet unit, but less than the number of platelets required to prepare two platelet units.

20. The method of claim 19, wherein the pooled platelet preparation comprises a sufficient number of platelets to prepare three platelet units.

21. The method of claim 19 or claim 20, wherein the plurality of platelet units comprises three platelet units.

22. The method of claim 10 or claim 11, wherein the first platelet donation comprises greater than the number of platelets required to prepare two platelet units, but less than the number of platelets required to prepare three platelet units, and the second platelet donation comprises greater than the number of platelets required to prepare two platelet units, but less than the number of platelets required to prepare three platelet units.

23. The method of claim 22, wherein the pooled platelet preparation comprises a sufficient number of platelets to prepare five platelet units.

24. The method of any one of claims 1-23, further comprising collecting a third platelet donation by apheresis from a third donor, combining the third platelet donation with the first and the second platelet donations to yield a pooled platelet preparation, and separating the pooled platelet preparation into a plurality of platelet units each in an individual container, wherein the plurality of platelet units comprises a greater number of platelet units than the total number of platelet units that can be prepared from the first platelet donation, the second platelet donation and the third platelet donation without combining the first, second and third platelet donations.

25. The method of any one of claims 1-24, wherein the second platelet donation is collected within 20 hours from the time the first platelet donation is collected.

26. The method of claim 25, wherein the second platelet donation is collected within 12 hours from the time the first platelet donation is collected.

27. The method of any one of claims 1-26, further comprising subjecting each of the first platelet donation and the second platelet donation to a pathogen inactivation process.

28. The method of any one of claims 1-26, further comprising subjecting the pooled platelet preparation to a pathogen inactivation process.

29. The method of claim 27 or claim 28, wherein subjecting the platelet donations or pooled platelet preparation to a pathogen inactivation process comprises treating the platelet donation or pooled platelet preparation with a pathogen inactivating compound.

30. The method of claim 29, wherein the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540.

31. The method of any one of claims 1-30, wherein the platelet units in the plurality of platelet units each comprise a therapeutic dose of platelets suitable for infusion into a human subject.

32. The method of any one of claims 1-31 wherein the platelet units each comprise at least about 2.4×10¹¹ platelets.

33. The method of claim 13, wherein the pooled platelet preparation comprises at least about 6.2×10¹¹ platelets.

34. The method of claim 14 or claim 15, wherein the pooled platelet preparation comprises at least about 9.3×10¹¹ platelets.

35. The method of any one of claims 13-18, wherein the platelet units each comprise a therapeutic dose of at least about 3×10¹¹ platelets.

36. The method of any one of claims 2, 4, 6, 8, 10, 12, 14, and 16, wherein targeting the platelet collection comprises one or more of determining an optimal platelet collection, providing an input to an apheresis system, and providing a donation volume to an apheresis system.

37. A method of preparing a plurality of platelet units suitable for infusion, comprising:
a) maintaining an inventory log of an inventory of apheresis-collected platelet preparations, the inventory log comprising for each platelet preparation a unique identifier, a donor blood type, a donation time and a quantity of platelets in the preparation,
b) identifying an available donor suitable for donating platelets by apheresis,
c) selecting from the inventory log at least one platelet preparation in the inventory for combining with platelets collected from the available donor,
d) collecting a new platelet donation by apheresis from the available donor,
e) combining the at least one selected platelet preparation and the new platelet donation to yield a pooled platelet preparation, and
f) separating the pooled platelet preparation into a plurality of platelet units each in an individual container, wherein the plurality of platelet units comprises a greater number of platelet units than the total number of platelet units that can be prepared from the at least one selected platelet preparation and the new platelet donation without combining the at least one selected platelet preparation and new platelet donation.

38. The method of claim 37, wherein the inventory of apheresis-collected platelet preparations comprises existing platelet preparations that have already been collected.

39. The method of claim 37 or claim 38, wherein the at least one selected platelet preparation comprises greater than the number of platelets required to prepare one platelet unit, but less than the number of platelets required to prepare two platelet units.

40. The method of any one of claims 37-39, wherein the at least one selected platelet preparation comprises at least about 125% of the number of platelets required to prepare one platelet unit, but less than the number of platelets required to prepare two platelet units.

41. The method of claim 37 or claim 38, wherein the at least one selected platelet preparation comprises greater than the number of platelets required to prepare two platelet units, but less than the number of platelets required to prepare three platelet units.

42. The method of any one of claims 37, 38, and 41, wherein the at least one selected platelet preparation comprises at least about 225% of the number of platelets required to prepare one platelet unit, but less than the number of platelets required to prepare three platelet units.

43. The method of claim 37 or claim 38, wherein the at least one selected platelet preparation comprises greater than the number of platelets required to prepare three platelet units, but less than the number of platelets required to prepare four platelet units.

44. The method of any one of claims 37, 38, and 43, wherein the at least one selected platelet preparation comprises at least about 325% of the number of platelets required to prepare one platelet unit, but less than the number of platelets required to prepare four platelet units.

45. The method of any one of claims 37-44, wherein collecting the new platelet donation comprises targeting the platelet collection to yield a donation comprising at least about 125% of the number of platelets required to prepare one platelet unit, but less than the number of platelets required to prepare two platelet units.

46. The method of any one of claims 37-44, wherein the new platelet donation comprises greater than the number of platelets required to prepare one platelet unit, but less than the number of platelets required to prepare two platelet units.

47. The method of any one of claims 37-44, wherein collecting the new platelet donation comprises targeting the platelet collection to yield a donation comprising at least about 225% of the number of platelets required to prepare one platelet unit, but less than the number of platelets required to prepare three platelet units.

48. The method of any one of claims 37-44, wherein the new platelet donation comprises greater than the number of platelets required to prepare two platelet units, but less than the number of platelets required to prepare three platelet units.

49. The method of any one of claims 37-44, wherein the new platelet donation comprises greater than the number of platelets required to prepare three platelet units, but less than the number of platelets required to prepare four platelet units.

50. The method of claim 39 or claim 40, wherein collecting the new platelet donation from the available donor comprises collecting a sufficient number of platelets to prepare three platelet units from the pooled preparation.

51. The method of claim 41 or claim 42, wherein collecting the new platelet donation from the available donor comprises collecting a sufficient number of platelets to prepare five platelet units from the pooled preparation.

52. The method of claim 43 or claim 44, wherein collecting the new platelet donation from the available donor comprises collecting a sufficient number of platelets to prepare seven platelet units from the pooled preparation.

53. The method of claim 45 or claim 46, wherein the at least one selected platelet preparation comprises greater than the number of platelets required to prepare one platelet unit, but less than the number of platelets required to prepare two platelet units, and the new platelet donation comprises greater than the number of platelets required to prepare one platelet unit, but less than the number of platelets required to prepare two platelet units.

54. The method of claim 53, wherein the plurality of platelet units comprises three platelet units.

55. The method of claim 47 or claim 48, wherein the at least one selected platelet preparation comprises greater than the number of platelets required to prepare two platelet units, but less than the number of platelets required to prepare three platelet units, and the new platelet donation comprises greater than the number of platelets required to prepare two platelet units, but less than the number of platelets required to prepare three platelet units.

56. The method of any one of claims 37-55, further comprising identifying a second available donor suitable for donating platelets by apheresis, collecting a second new platelet donation by apheresis from the second available donor, combining the second new platelet donation with the at least one selected platelet preparation and the new platelet donation to yield the pooled platelet preparation, and separating the pooled platelet preparation into a plurality of platelet units each in an individual container, wherein the plurality of platelet units comprises a greater number of platelet units than the total number of platelet units that can be prepared from the at least one selected platelet preparation, the new platelet donation and the second new platelet donation without combining the at least one selected platelet preparation, the new platelet donation and the second new platelet donation.

57. The method of any one of claims 37-56, wherein the new platelet donation is collected within 12 hours from the time the at least one selected platelet preparation is collected.

58. The method of any one of claims 37-57, further comprising subjecting each of the at least one selected platelet preparation and the new platelet donation to a pathogen inactivation process.

59. The method of claim 58, wherein subjecting each of the at least one selected platelet preparation and the new platelet donation to a pathogen inactivation process comprises treating each of the at least one selected platelet preparation and the new platelet donation with a pathogen inactivating compound.

60. The method of any one of claims 37-57, further comprising subjecting the pooled platelet preparation to a pathogen inactivation process.

61. The method of claim 60, wherein subjecting the pooled platelet preparation to a pathogen inactivation process comprises treating the pooled platelet preparation with a pathogen inactivating compound.

62. The method of claim 59 or claim 61, wherein the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540.

63. The method of any one of claims 37-62, wherein the platelet units in the plurality of platelet units each comprise a therapeutic dose of platelets suitable for infusion into a human subject.

64. The method of any one of claims 37-63, wherein the platelet units each comprise at least about 2.4×10¹¹ platelets.

65. The method of any one of claims 50-52, wherein the platelet units each comprise a therapeutic dose of at least about 3×10¹¹ platelets.

66. The method of claim 45 or 47, wherein targeting the platelet collection comprises one or more of determining an optimal platelet collection, providing an input to an apheresis system, and providing a donation volume to an apheresis system.

67. A platelet unit suitable for infusion prepared by the method of any one of claims 1-66.

68. A method of preparing two or more therapeutic dosage units of platelets comprising:
a) combining two or more apheresis-derived platelet donor units to form a pooled platelet composition,
b) subjecting the pooled platelet composition to a pathogen inactivation process, and
c) separating the pooled platelet composition into two or more therapeutic dosage units of platelets, each in an individual container.

69. The method of claim 68, wherein the number of therapeutic dosage units of platelets following step c) is greater than the number of therapeutic dosage units of platelets that can be prepared from the two or more apheresis-derived platelet donor units individually, without combining the two or more donor units.

70. The method of claim 68 or claim 69, wherein two apheresis-derived platelet donor units are combined and each donor unit comprises greater than the number of platelets required to prepare one therapeutic dosage unit of platelets, but less than the number of platelets required to prepare two therapeutic dosage units of platelets.

71. The method of claim 70, wherein each donor unit comprises at least about 125% of the number of platelets required to prepare one platelet unit.

72. The method of claim 68 or claim 69, wherein the two apheresis-derived platelet donor units are combined and each donor unit comprises greater than the number of platelets required to prepare two therapeutic dosage units of platelets, but less than the number of platelets required to prepare three therapeutic dosage units of platelets.

73. The method of any one of claims 68-72, wherein at least one of two or more apheresis-derived platelet donor units is selected from an inventory log of an inventory of apheresis-collected platelet donor units, the inventory log comprising for each platelet donor unit a unique identifier, a donor blood type, a donation time and a quantity of platelets in the donor unit.

74. The method of any one of claims 68-73, wherein the two or more apheresis-derived platelet donor units are combined within 12 hours from the time of collection.

75. The method of any one of claims 68-74, wherein subjecting the pooled platelet composition to a pathogen inactivation process comprises treating the pooled pathogen composition with a pathogen inactivating compound.

76. The method of claim 75, wherein the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540.

77. The method of any one of claims 68-76, wherein each therapeutic dosage unit of platelets comprises at least 2.4×10¹¹ platelets.

78. A therapeutic dosage unit of platelets prepared by the method of any one of claims 68-77.

79. A method for collecting a platelet donation by apheresis from a donor, comprising:
a) maintaining an inventory log of an inventory of apheresis-collected platelet preparations, the inventory log comprising for each platelet preparation a unique identifier, a donor blood type, a donation date and a quantity of platelets in the preparation,
b) identifying a donor suitable for donating platelets by apheresis,
c) selecting from the inventory log one or more platelet preparations suitable for combining with a new platelet donation to be collected by apheresis from the donor,
d) determining a target volume of the new platelet donation, wherein the target volume optimizes the number of platelet units that can be prepared from a combination comprising the new platelet donation and the one or more selected platelet preparations; and
e) collecting the target volume of the new platelet donation by apheresis from the donor.

80. The method of claim 79, further comprising:
f) combining the one or more selected platelet preparations and the new platelet donation to yield a pooled platelet preparation; and
g) separating the pooled platelet preparation into a plurality of platelet units each in an individual container.

81. The method of claim 80, wherein each of the platelet units in the plurality of platelet units comprises a therapeutic dose suitable for infusion into a human subject.

82. The method of claim 80 or claim 81, wherein the plurality of platelet units comprises a greater number of platelet units than the total number of platelet units that can be prepared from the one or more selected platelet preparations and the new platelet donation without combining the one or more selected platelet preparations and the new platelet donation.

83. The method of any one of claims 79-82, wherein the one or more selected platelet preparations comprises greater than the number of platelets required to prepare one platelet unit, but less than the number of platelets required to prepare two platelet units.

84. The method of any one of claims 79-82, wherein the one or more selected platelet preparations comprises at least about 125% of the number of platelets required to prepare one platelet unit, but less than the number of platelets required to prepare two platelet units.

85. The method of claim 83 or claim 84, wherein collecting the new platelet donation from the donor comprises collecting a sufficient number of platelets to prepare three platelet units from the pooled preparation.

86. The method of any one of claims 79-82, wherein the one or more selected platelet preparations comprises greater than the number of platelets required to prepare two platelet units, but less than the number of platelets required to prepare three platelet units.

87. The method of claim 86, wherein collecting the new platelet donation from the donor comprises collecting a sufficient number of platelets to prepare five platelet units from the pooled preparation.

88. The method of claim 81 or claim 82, wherein each of the one or more selected platelet preparations and the new platelet donation comprises greater than the number of platelets required to prepare one therapeutic dose, but less than the number of platelets required to prepare two therapeutic doses.

89. The method of claim 81 or claim 82, wherein each of the one or more selected platelet preparations and the new platelet donation comprises greater than the number of platelets required to prepare two therapeutic dosage units of platelets, but less than the number of platelets required to prepare three therapeutic dosage units of platelets.

90. The method of any one of claims 80-89, wherein the one or more selected platelet preparations and the new platelet donation are combined within 12 hours from the time of collection.

91. The method of any one of claims 80-90, wherein each platelet unit comprises at least 2.4×10¹¹ platelets.

92. The method of any one of claims 80-91, further comprising subjecting the pooled platelet preparation to a pathogen inactivation process.

93. The method of claim 92, wherein subjecting the pooled platelet preparation to a pathogen inactivation process comprises treating the pooled platelet preparation with a pathogen inactivating compound.

94. The method of any one of claims 80-91, further comprising subjecting each of the one or more selected platelet preparations and the new platelet donation to a pathogen inactivation process.

95. The method of claim 94, wherein subjecting the one or more selected platelet preparations and the new platelet donation to a pathogen inactivation process comprises treating the one or more selected platelet preparations and the new platelet donation with a pathogen inactivating compound.

96. The method of claim 93 or claim 95, wherein the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540.

97. A platelet unit suitable for infusion into a human subject, wherein the platelet unit is selected from a plurality of platelet units prepared by the method of any one of claims 80-96.

98. A therapeutic dosage unit of platelets suitable for infusion into a subject, wherein the therapeutic dosage unit comprises pooled apheresis-derived platelets from two or more donors, and wherein the pooled apheresis-derived platelets have been treated with a pathogen inactivating compound.

99. The therapeutic dosage unit of claim 98, wherein the apheresis-derived platelets have been treated with the pathogen inactivating compound prior to pooling.

100. The therapeutic dosage unit of platelets of claim 98, wherein the apheresis-derived platelets have been treated with the pathogen inactivating compound after pooling.

101. The therapeutic dosage unit of platelets of claims 98-100, wherein the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540.

102. The therapeutic dosage unit of platelets of any one of claims 98-101, wherein the therapeutic dosage unit comprises at least 2.4×10¹¹ platelets.

103. A method of infusing platelets into a subject in need thereof, comprising infusing into the subject a platelet unit of claim 67 or claim 97, or a therapeutic dosage unit of platelets of any one of claims 78 and 98-102.

104. A computer-implemented method for optimization of platelet units for infusion, comprising:
at a computer system including one or more processors and memory,
receiving one or more values associated with one or more properties of a platelet donation;
instructing to search, based on the received one or more values, a plurality of values associated with representations of a plurality of platelet preparations;
determining, based on the search results, a volume of the platelet donation; and
providing the determined volume of the platelet donation, wherein the determined volume of the platelet donation enables optimization of platelet units.

105. The method of claim 104, wherein at least one of the platelet donation and the plurality of platelet preparations comprises apheresis-derived platelets.

106. The method of claim 104 or 105, wherein the platelet donation comprises platelets being currently collected from a donor or platelets to be collected from a donor.

107. The method of claim 104 or claim 105, wherein the one or more properties of the platelet donation comprises: a blood type, a donation time, number of platelets per unit volume, and optionally a maximum platelet donation volume.

108. The method of any one of claims 104-107, wherein instructing to search the plurality of values associated with representations of a plurality of platelet preparations comprises:
obtaining one or more criteria for searching using the one or more received values associated with one or more properties of the platelet donation;
instructing to search the plurality of values associated with the representations of the plurality of platelet preparations based on the one or more criteria for searching; and
obtaining one or more candidate representations of platelet preparations from the representations of the plurality of platelet preparations, the one or more candidate representations of platelet preparations having values that satisfy the one or more criteria for searching.

109. The method of claim 108, wherein the criteria for searching include one or more threshold conditions of one or more of: a blood type, a donation time, a platelet volume and number of platelets per unit volume.

110. The method of claim 108 or claim 109, wherein obtaining the one or more candidate representations of platelet preparations comprises filtering the representations of the plurality of platelet preparations based on the one or more criteria for searching.

111. The method of any one of claims 104-110, wherein the representations of the plurality of platelet preparations include at least one of: platelet preparations stored in a platelet preparation inventory, platelet preparations being currently collected from a plurality of donors, and platelet preparations to be collected from a plurality of donors.

112. The method of any one of claims 104-111, wherein determining the volume of the platelet donation comprises:
obtaining one or more values corresponding to one or more candidate representations of platelet preparations provided by the search results;
obtaining one or more criteria for platelet units, the criteria for platelet units enabling optimization of platelet units;
determining, based on the one or more values corresponding to one or more candidate representations of platelet preparations and the one or more criteria for platelet units, one or more platelet preparations to select from the one or more candidate representations of the plurality of platelet preparations; and
obtaining the volume of the platelet donation based on the determined platelet preparation.

113. The method of any one of claims 104-112, wherein providing the determined volume of the platelet donation comprises at least one of:
displaying the determined volume of the platelet donation on a display of the computer system;
sending a notification providing the determining volume of the platelet donation; and
providing the determined volume of the platelet donation to an apheresis system.

114. The method of any one of claims 104-113, further comprising combining the platelet donation and one or more platelet preparations selected from the representations of the plurality of platelet preparations to yield a pooled platelet preparation.

115. The method of claim 114, further comprising separating the pooled platelet preparation into a plurality of platelet units each in an individual container.

116. A non-transitory computer readable medium having instructions stored thereon for optimization of platelet units for infusion, the instructions, when executed by one or more processors of a computer system, cause the computer system to:
receive one or more values associated with one or more properties of a platelet donation;
instruct to search, based on the received one or more values, a plurality of values associated with representations of a plurality of platelet preparations;
determine, based on the search results, a volume of the platelet donation; and
provide the determined volume of the platelet donation, wherein the determined volume of the platelet donation enables optimization of platelet units.

117. A system for optimization of platelet units for infusion, comprising:
one or more processors; and
memory having instructions stored thereon, the instruction, when executed by the one or more processors, cause the computer system to:
receive one or more values associated with one or more properties of a platelet donation;
instruct to search, based on the received one or more values, a plurality of values associated with representations of a plurality of platelet preparations;
determine, based on the search results, a volume of the platelet donation; and
provide the determined volume of the platelet donation, wherein the determined volume of the platelet donation enables optimization of platelet units.

118. The system of claim 117, wherein the computer system is integrated with an apheresis system.

119. A computer-implemented method for optimization of platelet units for infusion, comprising:
at a computer system including one or more processors and memory,
receiving one or more values associated with one or more properties of a first platelet preparation;
instructing to search, based on the received one or more values, a plurality of values associated with representations of a plurality of platelet preparations;
selecting, based on the search results, a second platelet preparation from the representations of a plurality of platelet preparations; and
providing the selected second platelet preparation, wherein the selected second platelet preparation enables optimization of platelet units.

120. The method of claim 119, wherein at least one of the first platelet preparation and the plurality of platelet preparations comprises apheresis-derived platelets.

121. The method of claim 119 or claim 120, wherein the first platelet preparation comprises a platelet preparation stored in a platelet preparation inventory.

122. The method of any one of claims 119-121, wherein the one or more properties of the first platelet preparation comprises: a blood type, a donation time, a volume, and number of platelets per unit volume.

123. The method of any one of claims 119-122, wherein instructing to search the plurality of values associated with representations of a plurality of platelet preparations comprises:
obtaining one or more criteria for searching using the one or more received values associated with one or more properties of the first platelet preparation;
instructing to search the plurality of values associated with the representations of the plurality of platelet preparations based on the one or more criteria for searching; and
obtaining one or more candidate representations of platelet preparations from the representations of the plurality of platelet preparations, the one or more candidate representations of platelet preparations having values that satisfy the one or more criteria for searching.

124. The method of claim 123, wherein the criteria for searching include one or more threshold conditions of one or more of: a blood type, a donation time, a volume, and number of platelets per unit volume.

125. The method of claim 123 or claim 124, wherein obtaining the one or more candidate representations of platelet preparations comprises filtering the representations of the plurality of platelet preparations based on the one or more criteria for searching.

126. The method of any one of claims 119-125, wherein the representations of the plurality of platelet preparations include at least one of: platelet preparations stored in a platelet preparation inventory, platelet preparations being currently collected from a plurality of donors, and platelet preparations to be collected from a plurality of donors.

127. The method of any one of claims 119-126, wherein selecting the second platelet preparation comprises:
obtaining one or more values corresponding to one or more candidate representations of platelet preparations provided by the search results;
obtaining one or more criteria for platelet units, the criteria for platelet units enabling optimization of platelet units; and
selecting, based on the one or more values corresponding to one or more candidate representations of platelet preparations and the one or more criteria for platelet units, a second platelet preparation from the one or more candidate representations of platelet preparations.

128. The method of any one of claims 119-127, wherein providing the selected second platelet preparation comprises at least one of:
displaying the selected second platelet preparation on a display of the computer system; and
sending a notification providing the selected second platelet preparation; and
providing an identification of the selected second platelet preparation to an apheresis system.

129. The method of any one of claims 119-128, further comprising combining the first platelet preparation and selected second platelet preparation to yield a pooled platelet preparation.

130. The method of claim 129, further comprising separating the pooled platelet preparation into a plurality of platelet units each in an individual container.

131. A non-transitory computer readable medium having instructions stored thereon for optimization of platelet units for infusion, the instructions, when executed by one or more processors of a computer system, cause the computer system to:
receive one or more values associated with one or more properties of a first platelet preparation;
instruct to search, based on the received one or more values, a plurality of values associated with representations of a plurality of platelet preparations;
select, based on the search results, a second platelet preparation from the representations of a plurality of platelet preparations; and
provide the selected second platelet preparation, wherein the selected second platelet preparation enables optimization of platelet units.

132. A system for optimization of platelet units for infusion, comprising:
one or more processors; and
memory having instructions stored thereon, the instruction, when executed by the one or more processors, cause the computer system to:
receive one or more values associated with one or more properties of a first platelet preparation;
instruct to search, based on the received one or more values, a plurality of values associated with representations of a plurality of platelet preparations;
select, based on the search results, a second platelet preparation from the representations of a plurality of platelet preparations; and
provide the selected second platelet preparation, wherein the selected second platelet preparation enables optimization of platelet units.

133. The system of claim 132, wherein the computer system is integrated with an apheresis system.

134. The method of any one of claims 114, 115, 129, and 130, further comprising subjecting the pooled platelet preparation to a pathogen inactivation process.

135. The method of claim 134, wherein subjecting the pooled platelet preparation to a pathogen inactivation process comprises treating the pooled platelet preparation with a pathogen inactivating compound.

136. The method of claim 135, wherein the pathogen inactivating compound is a photoactive pathogen inactivating compound selected from the group consisting of a psoralen, an isoalloxazine, an alloxazine, a phthalocyanine, a phenothiazine, a porphyrin, and merocyanine 540.

137. A processing set for preparing a plurality of platelet units from two or more platelet donations, wherein each of the platelet units is pathogen-inactivated and suitable for infusion into a subject, the processing set comprising:
a) a first container that contains a pathogen inactivation compound (PIC) and is suitable for combining the two or more platelet donations with the PIC;
b) a second container, coupled to the first container, within which the two or more platelet donations in admixture with the PIC can be photochemically inactivated under sterile conditions, wherein the volume of the two or more platelet donations is sufficient to yield the plurality of platelet units;
c) a compound absorption device (CAD) coupled to the second container such that the two or more photochemically inactivated platelet donations can be transferred from the second container to the compound absorption device under sterile conditions; and
d) three or more third containers, each of which is coupled to the compound absorption device such that the two or more photochemically inactivated platelet donations can be transferred from the compound absorption device to the three or more third containers under sterile conditions to provide the plurality of platelet units,
wherein the plurality of platelet units comprises a greater number of platelet units than the total number of platelet units that can be prepared from the two or more donations without combining the two or more platelet donations.

138. The processing set of claim 137, comprising three third containers, each of which is coupled to the compound absorption device such that the two or more photochemically inactivated platelet donations can be transferred from the compound absorption device to the three third containers under sterile conditions to provide the plurality of pathogen-inactivated platelet units.

139. The processing set of claim 137 or claim 138, further comprising an additional container suitable for containing one or more of the two or more platelet donations, wherein the additional container is coupled to the first container such that the one or more platelet donations of the additional container can be transferred from the additional container to the first container under sterile conditions.

140. The processing set of claim 139, wherein the additional container is suitable for containing a platelet donation obtained by apheresis.

141. The processing set of claim 139 or claim 140, wherein the additional container is suitable for sterile coupling with a second additional container.

142. The processing set of claim 141, wherein the second additional container is suitable for contains one or more of the two or more platelet donations, and wherein the additional container is suitable for sterile coupling with the second additional container such that the one or more platelet donations of the second additional container can be transferred from the second additional container to the additional container under sterile conditions.

143. The processing set of any one of claims 139-141, further comprising a second additional container suitable for containing one or more of the two or more platelet donations, wherein the second additional container is coupled to the additional container such that the one or more platelet donations of the second additional container can be transferred from the second additional container to the additional container under sterile conditions.

144. The processing set of any one of claims 141-143, wherein the additional container is suitable for containing a platelet donation obtained by apheresis from a first donor, and wherein the second additional container is suitable for containing a platelet donation obtained by apheresis from a second donor.

145. The processing set of any one of claims 137-144, wherein the PIC is a psoralen.

146. The processing set of claim 145, wherein the PIC is amotosalen.

147. A kit comprising (a) a processing set, and (b) instructions for using the processing set to prepare a plurality of platelet units from two or more platelet donations, wherein each of the platelet units is pathogen-inactivated and suitable for infusion into a subject, wherein the plurality of platelet units comprises a greater number of platelet units than can be prepared from the two or more platelet donations without combining the two or more platelet donations, and
wherein the processing set comprises:
1) a first container that contains a pathogen inactivation compound (PIC) and is suitable for combining the two or more platelet donations with the PIC;
2) a second container, coupled to the first container, within which the two or more platelet donations in admixture with the PIC can be photochemically inactivated under sterile conditions, wherein the volume of the two or more platelet donations is sufficient to yield the plurality of platelet units;
3) a compound absorption device (CAD) coupled to the second container such that the two or more photochemically inactivated platelet donations can be transferred from the second container to the compound absorption device under sterile conditions; and
4) three or more third containers, each of which is coupled to the compound absorption device such that the two or more photochemically inactivated platelet donations can be transferred from the compound absorption device to the three or more third containers under sterile conditions to provide the plurality of platelet units.

148. The kit of claim 147, comprising instructions for using the processing set to prepare a plurality of platelet units from two or more apheresis platelet donations, wherein each of the platelet units is pathogen-inactivated and suitable for infusion into a subject, wherein the plurality of platelet units comprises a greater number of platelet units than can be prepared from the two or more apheresis platelet donations without combining the two or more apheresis platelet donations.

149. The processing set of any one of claims 137-146 or the kit of claim 147 or claim 148, wherein the platelet units in the plurality of platelet units each comprise a therapeutic dose of platelets.
